(19) United States
(12) Patent Application Publication
(10) Pub. No.: US 2014/0031308 A1
(43) Pub. Date: Jan. 30, 2014

(54) BENCHMARKS FOR NORMAL CELL IDENTIFICATION

(71) Applicants: Longo Diane, Foster City, CA (US); Todd M. Covey, San Carlos, CA (US); David M. Soper, San Francisco, CA (US); Garry P. Nolan, San Francisco, CA (US); Alessandra Cesano, Redwood city, CA (US)

(72) Inventors: Longo Diane, Foster City, CA (US); Todd M. Covey, San Carlos, CA (US); David M. Soper, San Francisco, CA (US); Garry P. Nolan, San Francisco, CA (US); Alessandra Cesano, Redwood city, CA (US)

(21) Appl. No.: 13/821,539

(22) PCT Filed: Sep. 8, 2011
(86) PCT No.: PCT/US11/01565

§ 371 Date: Oct. 2, 2013

Related U.S. Application Data
(60) Provisional application No. 61/381,067, filed on Sep. 8, 2010.
(60) Provisional application No. 61/440,523, filed on Feb. 8, 2011.
(60) Provisional application No. 61/469,812, filed on Mar. 31, 2011.

(51) Int. Cl. G01N 33/50; G06F 19/12

(52) U.S. Cl. CPC G01N 33/5008; G06F 19/12
USPC 514/43; 435/29; 435/7.21; 703/11; 703/2

(57) ABSTRACT

Provided herein are methods, compositions, and kits for determining cell signaling profiles in normal cells and comparing the cell signaling profiles of normal cells to cell signaling profiles from a test sample.

### CROSS-REFERENCE

This application claims the benefit of U.S. Patent Application Nos. 61/381,067 filed Sep. 8, 2010; 61/440,523 filed Feb. 8, 2011; 61/469,812 filed Mar. 31, 2011; and 61/499,127 filed Jun. 20, 2011, which are incorporated by reference in their entireties.

### BACKGROUND OF THE INVENTION

Personalized medicine seeks to provide prognoses, diagnoses and other actionable medical information for an individual based on their profile of one or more biomarkers. Many of these diagnostics use classifiers which are binary statistical models trained to identify biomarkers which differentiate diseased cells from non-diseased cells (i.e., normal cells). While these classifiers are beneficial, a major drawback of these methods is that they only aim to determine similarity between two states: disease and normal. Often, disease states are heterogeneous, which complicates the identification of biomarkers to distinguish disease states and the development of these classifiers. For example, a classifier may classify an individual as having a normal profile as compared to one or more disease states even though the individual biomarker profile is different from the biomarker profile observed in normal cells. This is referred to as a ‘false negative’ identification. In order to fully eliminate false negative identifications, the classifier can model data representing all possible disease states. Since the heterogeneity of disease makes it difficult to obtain and characterize samples of all disease states, false negatives are inevitable.

Due to these limitations, in some instances it may be ideal to identify biomarkers to allow for the determination of similarity between cells from an individual and normal cells. Such a similarity comparison can benefit from the development of a statistical model that can characterize and distinguish normal cell data.

### SUMMARY OF THE INVENTION

In general, in one aspect, a method is provided comprising: a) identifying an activation level of one or more activatable elements in a first cell-type from a test sample; b) identifying an activation level of the one or more activatable elements in a second cell-type from a test sample; and c) determining a similarity value based on steps a) and step b) and a statistical model, wherein the statistical model specifies a range of activation levels of one or more activatable elements in the first cell-type and the second cell-type in a plurality of normal samples, wherein the statistical model further specifies the variance of the activation levels of the one or more activatable elements associated with cells in the plurality of normal samples. In one embodiment, identifying the activation level of the one or more activatable comprises: d) identifying the activation level of the one or more activatable elements in single cells derived from the test sample; e) identifying one or more cell-type markers in single cells derived from the test sample; and f) gating discrete populations of single cells based on the one or more cell-type markers associated with the single cells. In another embodiment, the method further comprises generating the statistical model, wherein generating the statistical model comprises: d) identifying the activation level of the one or more activatable elements in single cells derived from the plurality of normal samples; e) identifying one or more cell-type markers in single cells derived from the plurality of normal samples; f) gating cells in the plurality of normal samples based on the one or more cell-type markers associated with the single cells; and g) generating the statistical model that specifies the range of activation levels associated with cells in the normal samples.

In another embodiment, the statistical model further specifies the variance of activation levels of the one or more activatable elements associated cells in the plurality of normal samples. In another embodiment, the one or more activatable elements are selected from the group consisting of: pStat1, pStat3, pStat4, pStat5, pStat6 and p-p38. In another embodiment, the method further comprises contacting the test sample and the plurality of normal samples with one or more modulators. In another embodiment, the one or more modulators is selected from the group consisting of: G-CSM, EPO, GM-CSF, IFNa and IL-6.

In another embodiment, the test sample and the plurality of normal samples are derived from individuals with the same race, ethnicity, gender, or are in the same age-range. In another embodiment, the method further comprises normalizing the activation level of the one or more activatable elements in the first cell-type and the second cell-type based on a sample characteristic. In another embodiment, the sample characteristic comprises race, ethnicity, gender or age. In another embodiment, the identifying the activation level of the one or more activatable elements comprises flow cytometry. In another embodiment, the one or more activatable elements comprise one or more activatable elements from the plurality of normal samples that display variance of less than 50% of the activation level of the one or more activatable element in response to a modulator. In another embodiment, the similarity value is determined with a correlation metric or a fitting metric.

In another embodiment, the method further comprises displaying the activation level of the one or more activatable elements from the test sample and the plurality of normal samples in a report. In another embodiment, the displaying comprises a scatterplot, a line graph with error bars, a histogram, a bar and whisker plot, a circle plot, a radar plot, a heat map, and/or a bar graph.

In another embodiment, the method further comprises making a clinical decision based on the similarity value. In another embodiment, the clinical decision comprises a diagnosis, prognosis, or monitoring a subject from whom the test sample was derived.

In another embodiment, the one or more activatable elements comprises one or more proteins. In another embodiment, the identifying the activation level of the one or more activatable elements comprises contacting the one or more activatable elements with one or more binding elements. In another embodiment, the one or more binding elements comprises one or more phospho-specific antibodies. In another embodiment, the determining comprises use of a computer.

In another embodiment, the method further comprises administering a therapeutic agent to a subject from whom the test sample is derived based on the similarity value. In another embodiment, the method further comprises predicting a status of a second activatable element in a single cell from the test sample, wherein the second activatable element is different from the one or more activatable elements.

In another aspect, a method is provided comprising: a) identifying an activation level of two or more activatable elements in single cells from a test sample; b) obtaining a statistical model which specifies a range of activation levels of two more activatable elements in single cells in a plurality of samples used as a standard; and c) determining a similarity value between the activation levels in the single cells from a test sample and the statistical model. In one embodiment, the statistical model further specifies the variance of activation levels of the one or more activatable elements in single cells in the plurality of samples used as a standard. In another embodiment, the one or more activatable elements are selected from the group consisting of: pStat1, pStat3, pStat4, pStat5, pStat6 and p-p38. In another embodiment, the method further comprises contacting the test sample with one or more modulators. In another embodiment, the one or more modulators is selected from the group consisting of: G-CSM, EPO, GM-CSF, IL-27, IFNa and IL-6. In another embodiment, the test sample and the plurality of samples used as a standard are derived from individuals with the same race, ethnicity, gender, or are in the same age-range. In another embodiment, the method further comprises normalizing the activation level of the two or more activatable elements in single cells from the test sample based on a sample characteristic. In another embodiment, the sample characteristic comprises race, ethnicity, gender or age. In another embodiment, the identifying the activation level of the one or more activatable elements comprises flow cytometry. In another embodiment, the two or more activatable elements comprise one or more activatable elements from the plurality of samples used as a standard that display variance of less than 50% of the activation level of the one or more activatable elements in response to a modulator. In another embodiment, the similarity value is determined with a correlation metric or a fitting metric. In another embodiment, the method further comprises displaying the activation level of one or more of the two or more activatable elements from the test sample and the plurality of samples used as a standard in a report.

In another embodiment, the displaying comprises a scatterplot, a line graph with error bars, a histogram, a bar and whisker plot, a circle plot, a radar plot, a heat map, and/or a bar graph. In another embodiment, the method further comprises making a clinical decision based on the similarity value. In another embodiment, the clinical decision comprises a diagnosis, prognosis, or monitoring a subject from whom the test sample was derived. In another embodiment, the method further comprises administering a therapeutic agent to a subject from whom the test sample is derived based on the similarity value. In another embodiment, the method further comprises predicting the status of a second activatable element in a single cell from the test sample, wherein the second activatable element is different from the two or more activatable elements.

In another embodiment, the two or more activatable elements comprise two or more proteins. In another embodiment, the identifying the activation level of the two or more activatable elements comprises contacting the two or more activatable elements with one or more binding elements. In another embodiment, the one or more binding elements comprises one or more phosphospecific antibodies. In another embodiment, the determining comprises use of a computer.

In another aspect, a method of generating a normal cell profile is provided comprising obtaining a plurality of samples of cells from normal individuals, contacting the plurality of samples of cells from the normal individuals with one or more modulators, measuring an activation level of one or more activatable elements in the plurality of samples from the normal individuals, and generating a profile, wherein the profile comprises one or more ranges of the activation level of the one or more activatable elements from the plurality of samples of cells from the normal individuals.

In another embodiment, the profile comprises one or more ranges of activation levels of the one or more activatable elements that exhibit variance of less than 50% among normal samples. In another embodiment, the method further comprises gating each of the plurality of samples of cells from normal individuals into separate populations of cells. In another embodiment, the gating is based on cell surface markers. In another embodiment, the contacting comprises contacting the cells with a plurality of concentrations of the one or more modulators. In another embodiment, the measuring comprises measuring the activation level of the one or more activatable elements over a series of timepoints.

In another embodiment, the normal individuals have the same gender, race or ethnicity. In another embodiment, the normal individuals are selected based on the age of the normal individuals.

In another embodiment, the measuring the activation level of one or more activatable elements comprises flow cytometry. In another embodiment, the method further comprises displaying the activation level of the one or more activatable elements from the plurality of samples of cells from normal individuals in a report. In another embodiment, the displaying comprises a scatterplot, a line graph with error bars, a histogram, a bar and whisker plot, a circle plot, a radar plot, a heat map, and/or a bar graph.

In another embodiment, the one or more activatable elements comprises one or more proteins. In another embodiment, the measuring the activation level of the one or more activatable elements comprises contacting the one or more activatable elements with one or more binding elements. In another embodiment, the one or more binding elements comprises one or more phospho-specific antibodies. In another embodiment, the one or more activatable elements are selected from the group consisting of: pStat1, pStat3, pStat4, pStat5, pStat6 and p-p38. In another embodiment, the one or more modulators is selected from the group consisting of: G-CSM, EPO, GM-CSF, IL-27, IFNa and IL-6.

In another aspect, a method is provided comprising: a) measuring an activation level of one or more activatable elements from cells from a test sample from a subject; b) comparing the activation level of the one or more activatable elements from cells from the test sample to a model, wherein the model is derived from determining a range of activation levels of one or more activatable elements from samples of cells from a plurality of normal individuals; and c) preparing a report displaying the activation level of the one or activatable elements from the samples of cells from the plurality of normal individuals to the activation level of the one or more activatable elements from cells from the test sample from the subject.

In one embodiment, the samples of cells from the plurality of normal individuals were gated to separate populations of cells. In another embodiment, the method further comprises gating the sample of cells from the test sample from the subject into separate populations of cells. In another embodiment, the gating is based on one or more cell surface markers. In another embodiment, the samples of cells from a plurality of normal individuals were contacted with one or more modulators. In another embodiment, the method further comprises contacting the plurality of samples of cells from the test sample from the subject with the one or more modulators. In another embodiment, the normal individuals and the subject have the same gender, race, or ethnicity. In another embodiment, the method further comprises normalizing the activation level of the one or more activatable elements from cells form the test sample based on a sample characteristic. In another embodiment, the sample characteristic comprises race, ethnicity, gender or age. In another embodiment, the normal individuals are selected based on the age of the test subject. In another embodiment, the measuring the activation level of the one or more activatable elements comprises flow cytometry. In another embodiment, the displaying comprises a scatterplot, a line graph with error bars, a histogram, a bar and whisker plot, a circle plot, a radar plot, a heat map, and/or a bar graph. In another embodiment, the one or more activatable elements comprises one or more proteins. In another embodiment, the measuring an activation level of one or more activatable elements comprises contacting the one or more activatable elements with one or more binding elements. In another embodiment, the one or more binding elements comprises one or more phospho-specific antibodies. In another embodiment, the one or more activatable elements are selected from the group consisting of: pStat1, pStat3, pStat4, pStat5, pStat6 and p-p38. In another embodiment, the one or more modulators is selected from the group consisting of G-CSM, EPO, GM-CSF, IL-27, IFNa and IL-6.

In another embodiment, the method further comprises making a clinical decision based on said comparing. In another embodiment, the clinical decision comprises a diagnosis, prognosis, or monitoring the subject. In another embodiment, the method further comprises providing the report to a healthcare provider. In another embodiment, the method further comprises providing the report to the subject. In another embodiment, the report comprises information on cell growth, cell survival and/or cytostasis.

In another aspect, a report comprising a visual representation of multiparametric results of a test sample is provided, the visual representation comprising a comparison between an activation level of two or more activatable elements in single cells from a test sample and a range of activation levels of the two or more activatable elements in single cells in a plurality of samples used as a standard. In one embodiment, the report further comprises a statistical model, wherein the statistical model specifies the range of activation levels of the two or more activatable elements in single cells in a plurality of samples used as a standard. In another embodiment, the report further comprises a similarity value between the activation level of the two or more activatable elements in single cells from a test sample and the statistical model. In another embodiment, the report further comprises a scatterplot, a line graph with error bars, a histogram, a bar and whisker plot, a circle plot, a radar plot, a heat map, and/or a bar graph. In another embodiment, a computer server generates the report. In another embodiment, the report comprises information on cell growth, cell survival and/or cytostasis. In another embodiment, the two or more activatable elements comprise two or more proteins.

In another aspect, a method of preparing a report is provided comprising a) determining levels of two or more activatable elements in single cells obtained from a subject; b) comparing the levels of the two or more activatable elements to levels of the two or more activatable elements from a plurality of samples used as a standard; and c) preparing a report displaying the comparison. In one embodiment, the displaying comprises a scatterplot, a line graph with error bars, a histogram, a bar and whisker plot, a circle plot, a radar plot, a heat map, and/or a bar graph. In another embodiment, a computer server generates the report. In another embodiment, the report comprises information on cell growth, cell survival and/or cytostasis. In another embodiment, the two or more activatable elements comprise two or more proteins.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows boxplots for a range of signaling for each node in each population.

FIG. 2 illustrates some of the various cell-subpopulations which can be found in blood. For example, Naïve Helper T cells can be a sub-population of Helper T cells, T Cells, and Lymphocytes and can be distinct from Memory Cytotoxic T or Monocytes by their cell surface markers. Once the cell sub-populations are determined for each sample, the range of signaling of activatible elements can be statistically described. Note that the range of signaling for the particular activatible elements IFNa2.p-Stat1 and IL-6.p-Stat1 are different between Monocytes, Naïve Helper T cells, and Memory Cytotoxic T cells. These ranges of signaling which define normality within each cell population can then be quantified statistically, and disease state for a particular patient can be determined by comparison to these normal ranges of signaling.

FIG. 3 shows a schematic of an experiment for characterizing signal transduction networks implicated in the growth and survival of AML cells.

FIG. 4 shows that FLT3-ITD AML with high mutational load responses are more homogenous than FLT3-WT AML.

FIG. 5 shows that FLT3 WT donors are more heterogeneous than FLT3 ITD donors and show distinct patterns.

FIG. 6A shows signaling ranges for nodes within naïve cytotoxic T cells for D1 (darker boxplots) and D2 samples (lighter boxplots). FIG. 6 also shows cytokine signaling responses within the naïve cytototoxic T subset with significant age-associations in both datasets.

FIG. 7 shows (A) αIgD induced p-S6 signaling (based on the log₂fold increase in MFI in αIgD treated cells relative to the untreated control (0 min)) over time are shown for the African American (AA) and European American (EA) donors. The difference in p-S6 signaling (averaged over time points) between racial groups is statistically significant. (B) The percentage of CD20+ B cells that were IgD+ is shown for the AA and EA donors. The difference in IgD+ frequency between racial groups is statistically significant. In both (A) and (B), one of the ten donors was excluded due to an insufficient number (<200) of B cells collected for analysis.

FIG. 8 shows an embodiment of a cell signaling report. FIG. 8A is an overview of the report, and FIGS. 8B, 8C, 8D, 8E, and 8F show details of the report.

FIG. 9 shows another embodiment of a cell signaling report. FIGS. 9A, 9B, 9C, 9D, and 9E show different parts of the report.

FIG. 10A shows an overview of another embodiment of a cell signaling report. FIG. 10 shows signaling data: Stimulation time is 5-15 minutes. Kinase inhibitors when used were incubated on cells for 1 hr prior to stimulation. Radar plot axis is on a Log 2 scale. Cell growth assay: Cells were grown with the indicated conditions for 48 hours to characterize the dependence or independence on selected growth factors for cell survival and proliferation. Apoptosis/Cytostasis: After 48 hrs of growth phase in growth factors (FL, TPO, SCF, IL3), cells were incubated with drugs for 48 hrs. Abbreviations: p-, phospho; ERK, extracellular-signal-regulated kinase; S6, S6 Ribosome; STAT, Signal Transducers and Activators of Transcription; FL, FLT3 ligand; SCF, Stem Cell Factor; TPO, Thrombopoietin; TMZ, tomozolomide; AraC, cytarabine; K.I., kinase inhibitor; Topo. II, Topoisomerase II; HDAC, histone deacetylase; DNMT, DNA methyltransferase; GFs, growth factors; PARP, Poly (ADP-ribose) polymerase; JAK, Janus Kinase; MEK, Mitogen-activated protein kinase; PI3K, Phosphatidylinositol 3-kinase; mTor, mammalian target of rapamycin; HSP90, Heat Shock Protein 90. FIGS. 10B, 10C, 10D, 10E, 10F, 10G, 10H, 10I, 10J, and 10K show details of the report.

FIG. 11 shows normal PMBC DNA damage kinetics to double strand breaks induced by etoposide, Ara-C/Daunorubicin, and Mylotarg.

FIG. 12 shows normal PBMC Myeloid DNA Damage Kinetics to Double Strand Breaks induced by Etoposide, Ara-C/Daunorubicin, or Mylotarg.

FIG. 13 shows normal PBMC Lymph and Myeloid response to Ara-C/Daunorubicin: (kinetics and effect of Daunorubicin dose) measuring DNA Damage Response and Daunorubicin fluorescence.

FIG. 14 shows that AML samples can display a range of DDR responses compared to Normal Healthy Non-Diseased CD34+ Myeloblasts.

FIG. 15 shows SCNP results in healthy controls and MDS patients.

FIG. 16 illustrates a networked system for the remote acquisition or analysis of data obtained using methods described herein.

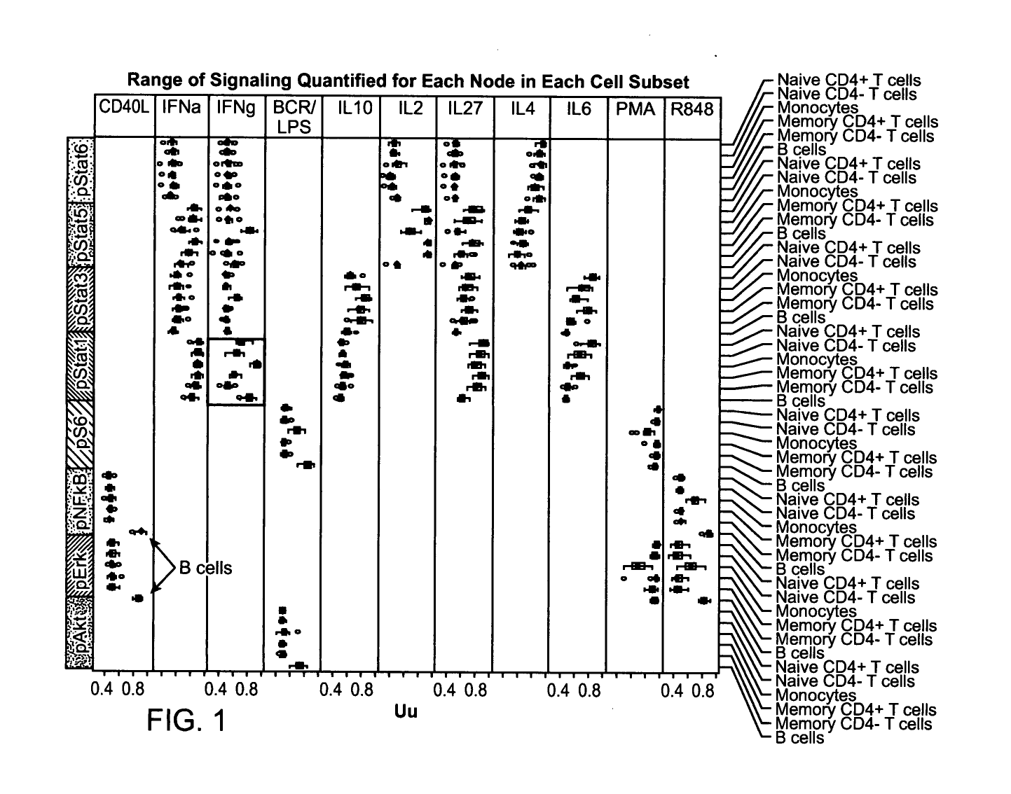

### DETAILED DESCRIPTION OF THE INVENTION

The methods, compositions, and kits disclosed herein incorporate information disclosed in other applications and texts. The following patent and other publications are hereby incorporated by reference in their entireties: Haskell et al, Cancer Treatment, 5^{th} Ed., W.B. Saunders and Co., 2001; Alberts et al., The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7^{th} Ed., Garland, and Leroith and Bondy, Growth Factors and Cytokines in Health and Disease, A Multi Volume Treatise, Volumes 1A and 1B, Growth Factors, 1996. Other conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y.; and Sambrook, Fritsche and Maniatis. “Molecular Cloning A laboratory Manual” 3rd Ed. Cold Spring Harbor Press (2001), all of which are herein incorporated in their entirety by reference for all purposes.

Patents and applications that are also incorporated by reference in their entirety include U.S. Pat. Nos. 7,381,535, 7,393,656, 7,695,924 and 7,695,926 and U.S. patent application Ser. Nos. 10/193,462; 11/655,785; 11/655,789; 11/655,821; 11/338,957; 12/877,998; 12/784,478; 12/730,170; 12/703,741; 12/687,873; 12/617,438; 12/606,869; 12/713,165; 12/293,081; 12/581,536; 12/776,349; 12/538,643; 12/501,274; 61/079,537; 12/501,295; 12/688,851; 12/471,158; 12/910,769; 12/460,029; 12/432,239; 12/432,720; 12/229,476, 12/877,998; 13/083,156; 61/469,812; 61/436,534; 61/317,187; and 61/353,155; and PCT Application Nos. PCT/US2011/029845 and PCT/US2010/048181.

Some commercial reagents, protocols, software and instruments that are useful in some embodiments are available at the Becton Dickinson Website http://www.bdbiosciences.com/features/products/, and the Beckman Coulter website, http://www.beckmancoulter.com/Default.asp?bhfv=7. Relevant articles include High-content single-cell drug screening with phosphospecific flow cytometry, Krutzik et al., Nature Chemical Biology, 23 Dec. 2007; Irish et al., FLt3 ligand Y591 duplication and Bcl-2 over expression are detected in acute myeloid leukemia cells with high levels of phosphorylated wild-type p53, Neoplasia, 2007; Irish et al. Mapping normal and cancer cell signaling networks: towards single-cell proteomics, Nature, Vol. 6 146-155, 2006; Irish et al., Single cell profiling of potentiated phospho-protein networks in cancer cells, Cell, Vol. 118, 1-20 Jul. 23, 2004; Schulz, K. R., et al., Single-cell phospho-protein analysis by flow cytometry, Curr Protoc Immunol, 2007, 78:8 8.17.1-20; Krutzik, P. O., et al., Coordinate analysis of murine immune cell surface markers and intracellular phosphoproteins by flow cytometry, J Immunol. 2005 Aug. 15; 175(4):2357-65; Krutzik, P. O., et al., Characterization of the murine immunological signaling network with phosphospecific flow cytometry, J Immunol. 2005 Aug. 15; 175(4):2366-73; Shulz et al., Current Protocols in Immunology 2007, 78:8.17.1-20; Stelzer et al. Use of Multiparameter Flow Cytometry and Immunophenotyping for the Diagnosis and Classification of Acute Myeloid Leukemia, Immunophenotyping, Wiley, 2000; and Krutzik, P. O. and Nolan, G. P., Intracellular phospho-protein staining techniques for flow cytometry: monitoring single cell signaling events, Cytometry A. 2003 October; 55(2):61-70; Hanahan D., Weinberg, The Hallmarks of Cancer, CELL, 2000 Jan. 7; 100(1) 57-70; Krutzik et al, High content single cell drug screening with phophosphospecific flow cytometry, Nat Chem Biol. 2008 February; 4(2):132-42. Experimental and process protocols and other helpful information can be found at http:/proteomices.stanford.edu. The articles and other references cited below are also incorporated by reference in their entireties for all purposes.

One embodiment described herein is a method for identifying ranges of activatable elements in different cell populations which can be used to characterize normal single cells. “Normal cells” or “healthy cells,” as referred to herein, can be cells that are not associated with any disease or pre-disease state. Normal cells or healthy cells can be used as a standard. Examples of activatable elements are described in detail below in the section entitled “Activatable Elements.” In some embodiments outlined in the examples below, the activatable elements are proteins that are phosphorylated in cell signaling pathways. In one embodiment, signaling response is measured based on the activation level or phosphorylation of the proteins involved in signaling pathways. Other types of activatable elements can be used to characterize normal single cells.

Normal can include the concept of a standard, which may be diseased state. A test sample can be compared to a standard. A parameter of a test sample, e.g., an activation level of an activatable element, can be adjusted or normalized based on a standard. A similarity value can be adjusted or normalized based on a standard.

In one embodiment, the observed activation levels of the activatable elements are induced by contacting the cells with one or more modulators (referred to herein as “stimulating the cells”). Modulators can be compounds or proteins that effect cell signaling. The cells can be contacted with different concentrations of one or more modulators to induce activation of the activatable elements. The amount by which the activatable element is induced by a modulator is referred to herein as its activation level. In one embodiment in the examples discussed below, the one or more modulators are used to induce phosphorylation of the activatable elements. In other embodiments one or more modulators may be used to induce other types of conformational or physical changes in activation elements. In the embodiments shown in the examples below the activation level of the activatable elements is characterized in single cells using multi-parametric flow cytometry. In other embodiments, other types of technology used to characterize activatable elements in single cells may be used (e.g., mass spectrometry, mass spectrometry-based flow cytometry). Some of these technologies are described below in the section entitled “Detection.”

The term “node” is used herein to describe a specific modulator/activatable element pair. Nodes can be represented using the notation modulator->activatable element. For example, IL-6->pStat5 represents the modulator IL-6 and the activatable element pStat5.

Characterization of activatable levels in normal single cells can have many benefits. First, understanding the range of activation levels in normal cells can provide valuable insight into the physiology of healthy cells, specifically the mechanisms by which healthy cells control signaling response(s). Second, establishing ranges of modulator-induced activation levels can allow for the identification of modulator-induced activation levels that are tightly controlled in healthy cells and therefore demonstrate little variance in healthy cells. The variance in activation level of an activatable element between two or more samples can be about, or less than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%. The fold difference in variance in activation level of an activatable element between two or more samples can be about, or less than about, 1.5, 2, 3, 4, 5, 6, 7, 8, 9, 10, 11, 12, 13, 14, 15, 16, 17, 18, 19, or 20-fold.

Different concentrations of modulators can be used to elicit different induced activation levels in healthy cells. Further, the activation levels induced by the modulators may be measured in single cells at different time points after modulation of the cells. Measuring the activation levels following modulation over time is discussed below in the section below entitled “Generation of Dynamic Activation State Data.” Measuring activation levels of nodes at different time points and using different concentrations of modulators can be beneficial as it can allow for a finer-resolution observation of the different activation responses of the cells to the modulators. As discussed with respect to the examples below, different concentrations of modulators can produce distinct activation levels at different time points. This resolution can allow for the identification of time points and/or concentrations of modulators that exhibit little variance and the observed ranges of activation levels can be used to distinguish and characterize normal cells.

Additionally, modeling the dynamic response of nodes over time can provide additional metrics that can be used to characterize the cells based on the activation levels over time (referred to herein as the “activation profile” of a node). For example, the activation profile may be used to generate metrics such as slope or can be expressed using linear equations. These metrics may also be used to characterize and distinguish normal single cells.

In some embodiments discussed below, the benefits of characterizing the ranges of activation levels in normal single cells are further enhanced by the segregation of single cells into discrete cell populations. A cell population can be a set of cells that share a common characteristic including but not limited to: cell type, cell morphology and expression of a gene or protein. Some analytical methods, such as multi-parametric flow cytometry, not only allow for the simultaneous measurement of activation levels of several activatable elements in single cells, but also allow for the measurement of other markers (e.g., cell surface proteins, activatable elements) that can be used to determine a type of the cell. These markers can be used in conjunction with gating methods (described below in the section entitled “Computational Identification of Cell Populations”) to segregate single cells into discrete cell sub-populations prior to analyzing the activation state data associated with the single cells.

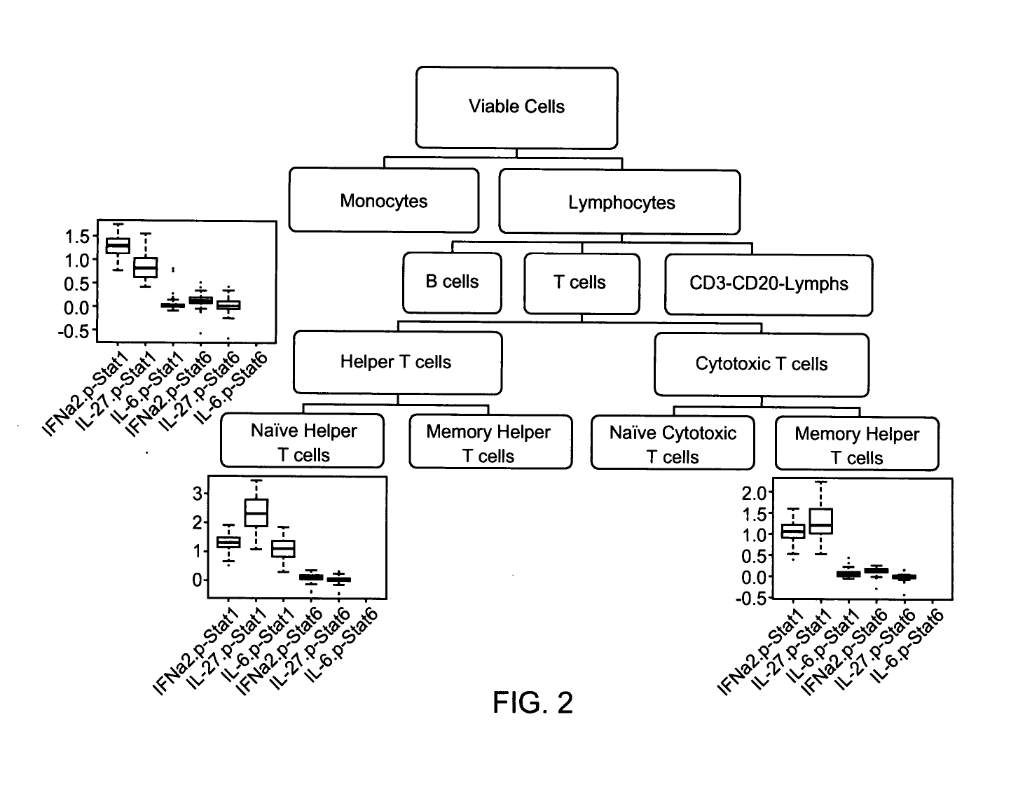

Once these cell sub-populations are identified the ranges of signaling of activatible elements can be quantified within each cell sub-population. The signaling ranges within each sub-population can then be described for normal and diseased states by statistical methods such as histograms, boxplots or otherwise. Multivariate statistical methods, such as regression, random forests, or clustering, may also be used to summarize the ranges of signaling across all cell sub-populations for normal and diseased states (See e.g., FIG. 2).

Cell signaling information for a subject, e.g., a patient, can be normalized based on a sample grouping or characteristic of the subject, e.g., race, gender, age, or ethnicity. The cell signaling information can be an activation level of one or more activation elements.

As demonstrated by the examples below, different cell populations exhibit different activation responses to modulators. By further segregating the cells based on the cell population, modulator-induced activation levels that distinguish and characterize normal cells can further be refined.

One embodiment of the invention is directed to methods for determining the status of an individual by determining an activation level of one or more activatable elements of cells in different discrete populations of cells obtained from the individual. Typically, the status of an individual can be a status related to the health of the individual (referred to herein as “health status” or “disease status”), but any type of status can be determined if it can be correlated to the status of cells (e.g., single cells) from one or more discrete populations of cells from the individual. In some embodiments, provided herein are methods for determining the status of an individual by creating a response panel using two or more discrete cell populations. In some embodiments, the status of an individual is determined by a method comprising: a) contacting a first cell from a first discrete cell population from said individual with at least a first modulator; b) contacting a second cell from a second discrete cell population from said individual with at least a second modulator; d) determining an activation level of at least one activatable element in said first cell and said second cell; e) creating a response panel for said individual comprising said determined activation levels of said activatable elements; and f) making a decision regarding the status of said individual, wherein said decision is based on said response panel.

Thus, provided herein are methods for the determination of the status of an individual by analyzing a plurality (e.g., two or more) of discrete populations of cells. In some embodiments, provided herein are methods to demarcate discrete populations of cells that correlate with a clinical outcome for a disease. In some embodiments, the methods provided herein use different discrete populations of cells, the analysis of which, in combination, allows for the determination of a status of an individual. In some embodiments, the methods provided herein use different discrete populations of cells the analysis of which, in combination, allows for the determination of the state of a cellular network. In some embodiments, provided herein are methods for the determination of a causal association between discrete populations of cells, where the causal association is indicative of the status of a cell network. In another embodiment, provided herein are methods to determine whether one or more cell populations that are part of a cellular network are associated with a status.

The status of an individual can be associated with a diagnosis, prognosis, choice or modification of treatment, and/or monitoring of a disease, disorder, or condition. Through the determination of the status of an individual, a health care practitioner can assess whether the individual is in the normal range for a particular condition or whether the individual has a pre-pathological or pathological condition warranting monitoring and/or treatment. Thus, in some embodiments, the status of an individual involves the classification, diagnosis, prognosis of a condition or outcome after administering a therapeutic to treat the condition.

One embodiment of the methods provided herein involves the classification, diagnosis, prognosis of a condition or outcome after administering a therapeutic to treat the condition. Another embodiment of the methods described herein involves monitoring and predicting an outcome of a condition. Another embodiment is drug screening using some of the methods described herein to determine which drugs may be useful in particular conditions. In some embodiments, an analysis method involves evaluating cell signals and/or expression markers in different discrete cell populations in performing these processes. One embodiment of cell signal analysis involves the analysis of one or more phosphorylated proteins (e.g., by flow cytometry) in different discrete cell populations. The classification, diagnosis, prognosis of a condition and/or outcome after administering a therapeutic to treat the condition is then determined based in the analysis of the one or more phosphorylated proteins in different discrete cell populations. In one embodiment, a signal transduction-based classification of a condition can be performed using clustering of phospho-protein patterns or biosignatures of the different cell discrete populations.

In some embodiments, a treatment is chosen based on a characterization of a plurality of discrete cell populations. In some embodiments, characterizing a plurality of discrete cell populations comprises determining the activation state of one or more activatable elements in the plurality of cell populations. The activatable element(s) analyzed among the plurality of discrete cell populations can be the same or can be different.

In some embodiments, provided herein are methods for classification, diagnosis, prognosis of a condition or outcome after administering a therapeutic to treat the condition by characterizing one or more pathways in different discrete cell populations. In some embodiments, a treatment is chosen based on the characterization of the pathway(s) simultaneously in the different discrete cell populations. In some embodiments, characterizing one or more pathways in different discrete cell populations comprises determining whether apoptosis pathways, cell cycle pathways, signaling pathways, or DNA damage pathways are functional in the different discrete cell populations based on the activation levels of one or more activatable elements within the pathways, where a pathway is functional if it is permissive for a response to a treatment.

In some embodiments, the characterization of different discrete cell populations in a condition (e.g., cancer) shows disruptions in cellular networks that are reflective of increased proliferation, increased survival, evasion of apoptosis, insensitivity to anti-growth signals and other mechanisms. In some embodiments, the disruption in these networks can be revealed by exposing a plurality of discrete cell populations to one or more modulators that mimic one or more environmental cues. For example, without intending to be limited to any theory, several different cell types participate as part of the immune system, including B cells, T cells, macrophages, neutrophils, basophils and eosinophils. Each of these cell types has a distinct role in the immune system, and communicates with other immune cells using secreted factors called cytokines, including interleukins, TNF, and the interferons. Macrophages phagocytose foreign bodies and are antigen-presenting cells, using cytokines to stimulate specific antigen dependent responses by B and T cells and non-specific responses by other cell types. T cells secrete a variety of factors to coordinate and stimulate immune responses to specific antigen, such as the role of helper T cells in B cell activation in response to antigen. The proliferation and activation of eosinophils, neutrophils and basophils respond to cytokines as well. Cytokine communication is often local, within a tissue or between cells in close proximity. Each of the cytokines is secreted by one set of cells and provokes a response in another target set of cells, often including the cell that secretes the cytokine.

In response to tissue injury, a multifactorial network of chemical signals can initiate and maintain a host response designed to heal the afflicted tissue. When a condition such as cancer is present in an individual the homeostasis in, e.g., tissue, organ and/or microenvironment is perturbed. For example, neoplasia-associated angiogenesis and lymphangiogenesis produces a chaotic vascular organization of blood vessels and lymphatics where neoplastic cells interact with other cell types (mesenchymal, haematopoietic and lymphoid) and a remodelled extracellular matrix. Neoplastic cells produce an array of cytokines and chemokines that are mitogenic and/or chemoattractants for granulocytes, mast cells, monocytes/macrophages, fibroblasts and endothelial cells. In addition, activated fibroblasts and infiltrating inflammatory cells can secrete proteolytic enzymes, cytokines and chemokines, which can be mitogenic for neoplastic cells, as well as endothelial cells involved in neoangiogenesis and lymphangiogenesis. These factors can potentiate tumor growth, stimulate angiogenesis, induce fibroblast migration and maturation, and enable metastatic spread via engagement with either the venous or lymphatic networks. Thus, determining the activation state data of various cell populations in an individual can provide a better picture of the status of the individual and/or the state of the cellular network.

In a condition like rheumatoid arthritis (RA), contributions made by interactions between dendritic cells, T cells and other immune cells, and local production of cytokines and chemokines may contribute to the pathogenesis of RA. These cells can further interact with local cells (e.g., synoviocytes). In response to local inflammation and production of proinflammatory cytokines, after unknown event dendritic cells, T cells and other immune cells can be attracted to the synovium in response to local production of cytokines and chemokines. In some patients with rheumatoid arthritis, chronic inflammation leads to the destruction of the cartilage, bone, and ligaments, causing deformity of the joints. Damage to the joints can occur early in the disease and be progressive.

The determination of the status (e.g., health status, disease status and/or any status indicating the pathophysiology of an individual) may also indicate response of an individual to treatment for a condition. Such information can allow for ongoing monitoring of the condition and/or additional treatment. In one embodiment, provided herein are methods for the detection of the presence of disease-associated cells or the absence or reduction of cells necessary for normal physiology in an individual that is being treated, or was previously treated, for the disease or condition. In some embodiments, the status may also indicate predicted response to a treatment.

In some embodiments, the determination of the status of an individual may be used to ascertain whether a previous condition or treatment has induced a new pre-pathological or pathological condition that requires monitoring and/or treatment. For example, treatment for many forms of cancers (e.g., lymphomas and childhood leukemias) can induce certain adult leukemias, and the methods described herein can allow for the early detection and treatment of such leukemias.

In a further embodiment, the status of an individual can indicate an individual's immunologic status and can reflect a general immunologic status, an organ or tissue specific status, or a disease related status.

Cells respond to environmental and systemic signals to adjust their responses to varying demands. For example, cells respond to factors such as hormones, growth factors and cytokine produced by other cells or from the environment. Cells also respond to injury and physiological changes. As a result, each tissue, organ, microenvironment (e.g., niche) or cell has the capacity to modulate the activity of cells. In addition, the presence of cells (e.g. cancer cells) can have influence in a surrounding tissue, organ, microenvironment (e.g., niche) or cell.

A cell might be passive in the communication with a surrounding tissue, organ, microenvironment (e.g., niche) or cell, merely adjusting their activity levels according to the environment demands. A cell might influence a surrounding tissue, organ, microenvironment (e.g., niche) or cell by virtue of progeny or signals such as cell contacts, secreted or membrane bounds factors. Thus, cells co-exist with other types of cells in a complex environment milieu. Different types of cells that interact with each other in a tissue, an organ, or a microenvironment such as a niche participate in a network that might determine the status of an individual (e.g., developing of a condition or performing normal functions).

A discrete cell population, as used herein, can refer to a population of cells in which the majority of cells is of a same cell type or has a same characteristic. For many years, research into several conditions (e.g., cancer) has focused on attempts to identify a causative cell population comprised of cells of a single cell type. However, several discrete cell populations or the interactions between several cell populations may contribute to the pathology of a condition. For example, in the case of a cancer cell, the cancer cell may possess a dysregulated response to an environmental cue (e.g., cytokine) such that the cell proliferates rather than undergo apoptosis. Alternatively, the environment in which the cell is located (e.g. niche, tissue, organ) may abnormally produce a factor that causes the cancer cell to undergo uncontrolled proliferation. In addition, the cancer cell may produce one or more factors that influence its environment (e.g. niche, tissue, organ), and, as a result the pathology of the cancer is worsened.

Thus, the successful diagnosis of a condition and use of therapies may require knowledge of the activation state data of different discrete cell populations that may play a role in the pathogenesis of a condition (e.g., cancer). The determination of the activation state data of different discrete cell populations that might interact directly or indirectly in a network serves as an indicator of the state of the network. In addition, it provides directionality to the interactions among the different discrete cell populations in the network. It also provides information across the cell populations participating in the network. As a result, the determination of activation state data of different discrete cell populations can serve as an indicator of a condition.

In some embodiments, the activation state data of a plurality of populations of cells is determined by analyzing multiple single cells in each population (e.g. by flow cytometry). Measuring multiple single cells in each discrete cell population in an individual provides multiple data points that in turn allows for the determination of the network boundaries in the individual. Measuring modulated networks at a single cell level provides the lever of biologic resolution that allows the assessment of intrapatient clonal heterogeneity ultimately relevant to disease management and outcome. The network boundaries and/or the state of the network might change when the individual is suffering from a pathological condition or if the individual is responding or not responding to treatment. Thus, the determination of network boundaries and/or the state of the network can be used for diagnosis, prognosis of a condition or determination of outcome after administering a therapeutic to treat the condition.

Provided herein are methods for determining the status of an individual by analyzing different discrete cell populations in said individual. In some embodiments, provided herein are methods for determining the state of a cellular network. The cellular network can be correlated with the status of an individual. In some embodiments, determining the status of an individual involves the classification, diagnosis, prognosis of a condition or outcome after administering a therapeutic to treat the condition.

The methods provided herein can be used to determine a range of activation levels of one or more activation elements. In some embodiments, the activation level of a first activatable element correlates with the activation level of a second activatable element. In some embodiments, the correlation is a positive correlation; in some embodiments, the correlation is a negative correlation. In some embodiments, an activation level of a plurality of activatable elements is determined. In some embodiments, the activation level of a first subset of one or more activatable elements is determined in a test sample, and the activation level of a second subset of one or more activatable elements is predicted based on known correlations between the first subset of one or more activatable elements and the second subset of activatable elements.

### Generating a Statistical Model of Induced Activation in Normal Cells

In one embodiment, the methods described herein allow for the identification of one or more activation levels that can be used to characterize normal cells. The one or more activation levels may be used to generate a statistical model that can be used to determine whether a cell associated with a test subject (e.g., an undiagnosed individual) exhibits a cell profile that is comparable to a profile for a normal cell.

Multiple methods can be used to determine the activation state of a cell, but, in one specific embodiment, samples of normal cells are treated with one or more modulators at a variety of different concentrations. The activation levels of a set of activation elements can be measured at a number of pre-defined time intervals using flow cytometry or other comparable techniques for measuring activation levels in single cells. In some embodiments, markers or their levels can be used to segregate the activation elements into discrete cell populations. The activation profiles for each cell population can be analyzed to identify one or more ranges of activation levels that exhibit little variance among the cell populations of normal samples. The activation profiles can be further analyzed to identify activation levels associated with different time points and/or modulator concentrations that are unique to a population of cells. The activation profiles can be further analyzed to identify slopes or other dynamic characteristics of the activation profiles that either exhibit little variance and/or are unique to a population of cells.

In some embodiments, activation state data (e.g., activation levels and/or activation profiles) derived from the normal cells can be used to determine the similarity between the normal cells and one or more samples derived from test subjects (e.g., individuals with unknown medical status; e.g., undiagnosed individuals). In these embodiments, the activatable elements from normal cells can be measured in a sample from a test subject (e.g., an undiagnosed individual).

In other embodiments, all activation state data derived from the normal samples is used to generate a statistical model including the range of observed activation levels in normal cells and the associated variance. The activation state data for a test subject (e.g., an undiagnosed individual) can be compared to the model of all the data, regardless of the level of variance and uniqueness of the activation state data. The activation state data may be compared using a correlation metric, a fitting metric or any other value that can be used to represent similarity to a range of values.

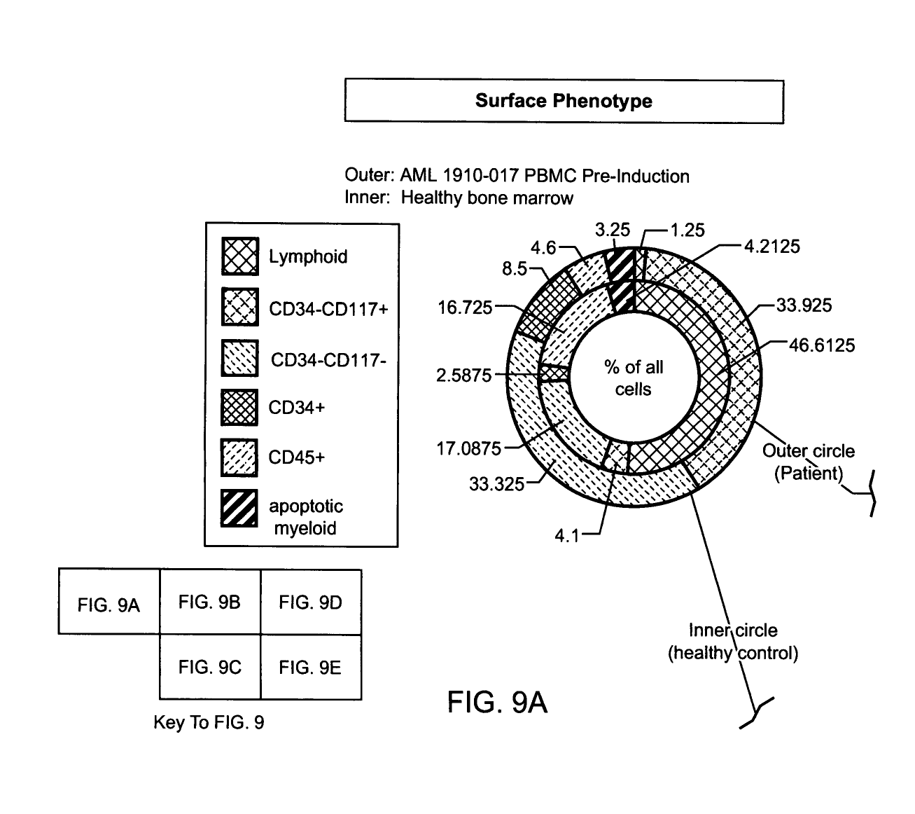

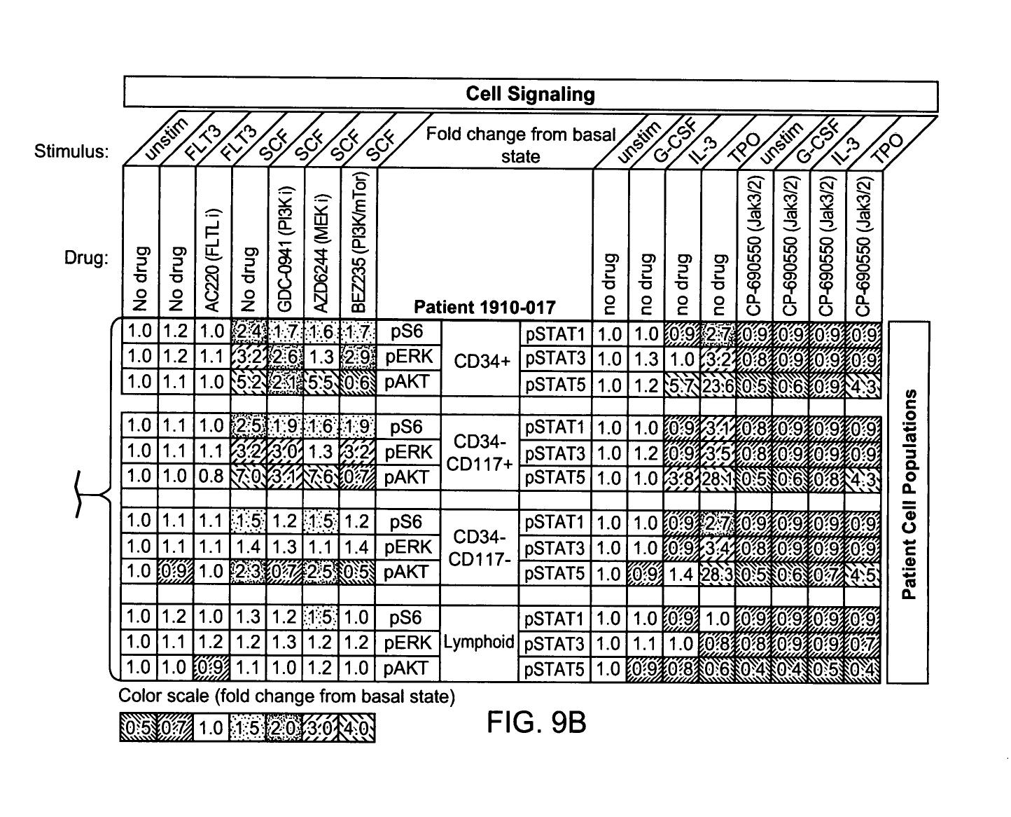

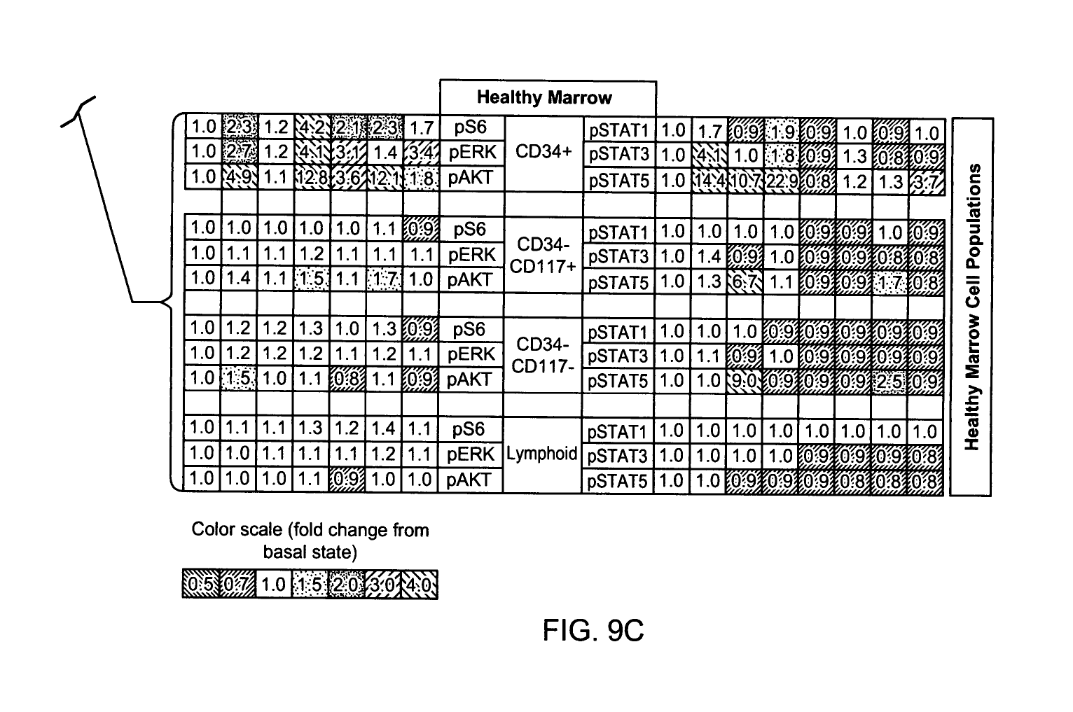

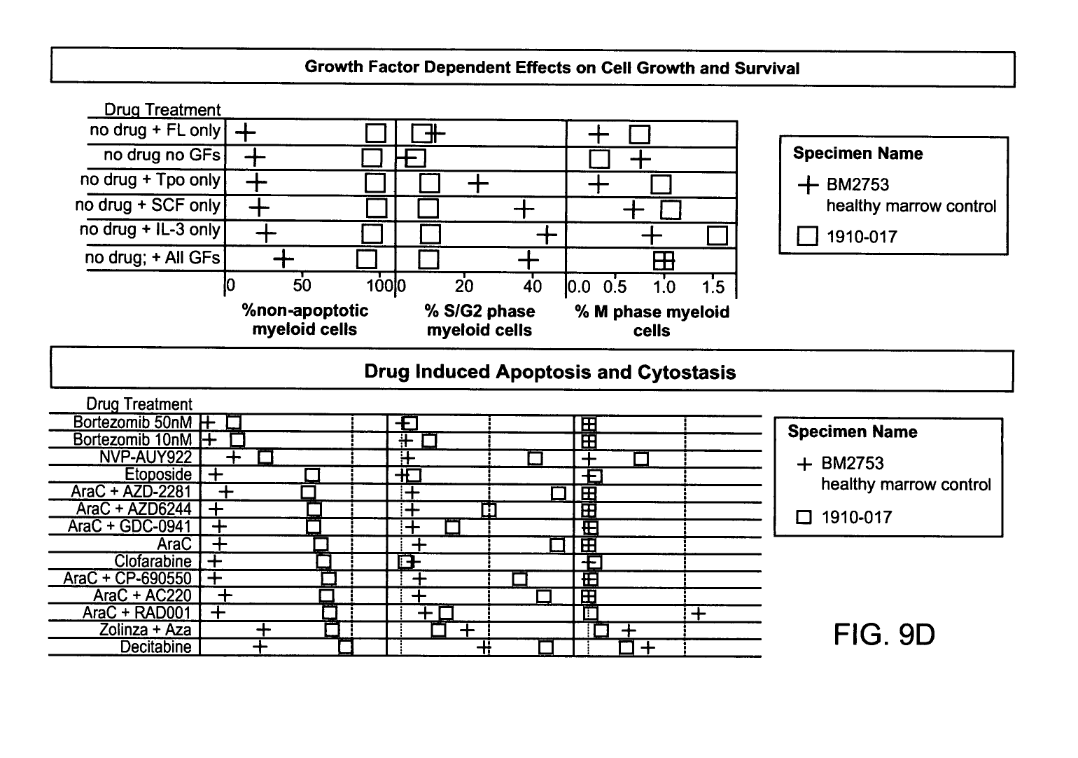

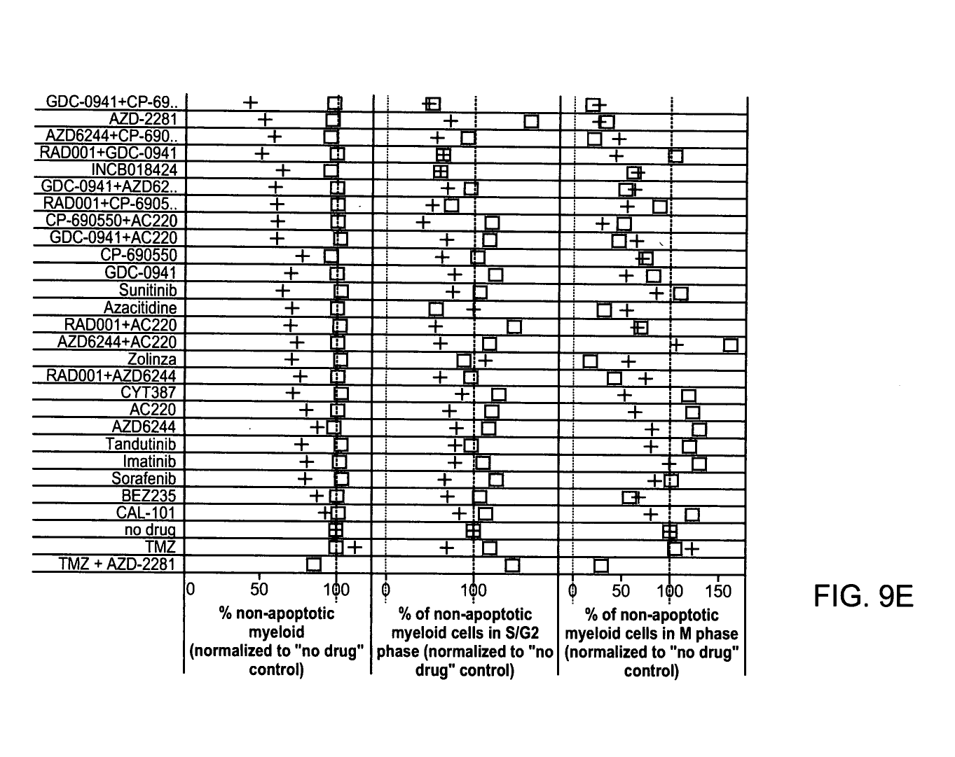

In some embodiments, the activation state data for a test subject (e.g., an undiagnosed individual) is plotted alongside data that represent the range of activation levels observed in normal cells. The range of activation levels observed in normal cells may be displayed or plotted as a scatterplot, a line graph with error bars, a histogram, a bar and whisker plot, a radar plot, and/or a bar graph for example. In some embodiments, activation state data for a test subject (e.g., an undiagnosed individual) is depicted in a heat map alongside data that represent the activation levels observed in normal cells. See FIGS. 9B and 9C for an example of a heat map. In some embodiments, correlations between nodes in different cell populations are illustrated using a circular plot, where nodes with a positive correlation (e.g., >0.5) are connected by a line of one color and nodes with a negative correlation (e.g., ≦−0.5) are connected by a line of a different color.

In some embodiments, the relative distribution of the cells into discrete cell populations is used to determine the similarity between the test subject (e.g., an undiagnosed individual) and normal cells. In these embodiments, the normal samples are analyzed to determine the relative percentages of the different cell populations. From these data, a range of percentages of cell populations can be derived. Using the range of observed values and the variance in the observed values, a metric that indicates similarity and a confidence interval may be produced. In one embodiment, the similarity value represents the overall similarity of the distribution over the different cell populations to the distribution observed in the normal samples and the confidence interval represents the probability of observing such similarity based on the distributions observed in the normal samples. This similarity value may be calculated independently from the similarity value calculated based on the activation levels or may be calculated in combination with the similarity value calculated based on the activation levels. This similarity value can indicate how similar the distribution of cell-types in a test sample are to the range of percentages of cell-types in normal samples.

In one embodiment, activation state data associated with the normal samples may be combined with data derived from samples that are known to be associated with disease states in order to generate a traditional binary or multi-class classifier. This classifier may be used experimentally to identify activation levels that distinguish the disease state from normal cells. This classifier may also be used to perform diagnoses of specific diseases. In a specific embodiment, activation state data from samples from normal individuals may be generated, analyzed and sold to various medical test developers for this purpose.

In some embodiments, methods described herein, comparison of data from normal cells to data from cells from a test subject (e.g., an undiagnosed subject), can be used for drug screening, diagnosis, prognosis, or prediction of disease treatment. In some embodiments, the methods described herein can be used to measure signaling pathway activity in single cells, identify signaling pathway disruptions in diseased cells, including rare cell populations, identify response and resistant biological profiles that guide the selection of therapeutic regimens, monitor the effects of therapeutic treatments on signaling in diseased cells, or monitor the effects of treatment over time. In some embodiments, the methods provided herein can enable biology-driven patient management and drug development, improve patient outcome, reduce inefficient uses of resources, and improve speed of drug development cycles.

### Modulators

In some embodiments, the methods and compositions utilize a modulator. A modulator can be an activator, a therapeutic compound, an inhibitor or a compound capable of impacting a cellular pathway. Modulators can also take the form of environmental cues and inputs. Modulators can be uncharacterized or characterized as known compounds. A modulator can be a biological specimen or sample of a cellular or physiological environment from an individual, which may be a heterogeneous sample without complete chemical or biological characterization. Collection of the modulator specimen may occur directly from the individual, or be obtained indirectly. An illustrative example would be to remove a cellular sample from the individual, and then culture that sample to obtain modulators.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators. In some embodiments, cells are exposed to 1-10, 1-7, 1-5, 2-10, 2-7, or 2-5 modulators. See U.S. Patent Application 61/048,657 which is incorporated by reference.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from about 0.0001% to 30%, about 0.001% to 30%, about 0.01% to 30%, about 0.1% to 30% or 1% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, drugs, immune modulators, ions, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absence of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element. A modulator can include, e.g., a psychological stressor.

In some embodiments the modulator is selected from the group consisting of growth factors, cytokines, adhesion molecules, drugs, hormones, small molecules, polynucleotides, antibodies, natural compounds, lactones, chemotherapeutic agents, immune modulators, carbohydrates, proteases, ions, reactive oxygen species, peptides, and protein fragments, either alone or in the context of cells, cells themselves, viruses, and biological and non-biological complexes (e.g., beads, plates, viral envelopes, antigen presentation molecules such as major histocompatibility complex). In some embodiments, the modulator is a physical stimuli such as heat, cold, UV radiation, and radiation. Examples of modulators, include but are not limited to SDF-1α, IFN-α, IFN-γ, IL-10, IL-6, IL-27, G-CSF, FLT-3L, IGF-1, M-CSF, SCF, PMA, Thapsigargin, H₂O₂, etoposide, AraC, daunorubicin, staurosporine, benzyloxycarbonyl-Val-Ala-Asp (OMe) fluoromethylketone (ZVAD), lenalidomide, EPO, azacitadine, decitabine, IL-3, IL-4, GM-CSF, EPO, LPS, TNF-α, and CD40L. In some embodiments, the modulator is a chemokine, e.g., CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXC12, CXCL13, CXCL14, CXCL15, CXCL16, CXCL17, XCL1, XCL2, or CX3CL1. In some embodiments, the modulator is an interleukin, e.g., IL-1 alpha, IL-1 beta, IL-2, IL-3, IL-4, IL-5, IL-6 (BSF-2), IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33 or IL-35.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor. In some embodiments cells are exposed to 1-10, 1-7, 1-5, 2-10, 2-7, or 2-5 modulators, where at least one of the modulators is an inhibitor.

In some embodiments, the inhibitor is an inhibitor of a cellular factor or a plurality of factors that participates in a cellular pathway (e.g., signaling cascade) in the cell. In some embodiments, the inhibitor is a phosphatase inhibitor. Examples of phosphatase inhibitors include, but are not limited to H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenylarsine oxide, Pyrrolidine Dithiocarbamate, and Aluminium fluoride. In some embodiments, the phosphatase inhibitor is H₂O₂.

In some embodiments, the activation level of an activatable element in a cell is determined by contacting the cell with at least 2, 3, 4, 5, 6, 7, 8, 9, or 10 modulators. In some embodiments, the activation level of an activatable element in a cell is determined by contacting the cell with at least 2, 3, 4, 5, 6, 7, 8, 9, or 10 modulators where at least one of the modulators is an inhibitor. In some embodiments the activation level of an activatable element in a cell is determined by contacting the cell with 1-10, 1-7, 1-5, 2-10, 2-7, or 2-5 modulators. In some embodiments, the activation level of an activatable element in a cell is determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

In some embodiments, the physiological status of a population of cells is determined by measuring the activation level of an activatable element when the population of cells is exposed to one or more modulators. The population of cells can be divided into a plurality of samples, and the physiological status of the population can be determined by measuring the activation level of at least one activatable element in the samples after the samples have been exposed to one or more modulators. In some embodiments, the physiological status of different populations of cells is determined by measuring the activation level of an activatable element in each population of cells when each of the populations of cells is exposed to a modulator. The different populations of cells can be exposed to the same or different modulators. In some embodiments, the modulators include H₂O₂, PMA, SDF1α, CD40L, IGF-1, IL-7, IL-6, IL-10, IL-27, IL-4, IL-2, IL-3, thapsigardin and/or a combination thereof. For instance, a population of cells can be exposed to one or more, all, or a combination of the following combination of modulators: H₂O₂ PMA; SDF1α; CD40L; IGF-1; IL-7; IL-6; IL-10; IL-27; IL-4; IL-2; IL-3; thapsigardin. In some embodiments, the physiological status of different populations of cells is used to determine the status of an individual as described herein. In some embodiments, the modulator is a chemokine, e.g., CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1, CXCL2, CXCL3, CXCL4, CXCL5, CXCL6, CXCL7, CXCL8, CXCL9, CXCL10, CXCL11, CXC12, CXCL13, CXCL14, CXCL15, CXCL16, CXCL17, XCL1, XCL2, or CX3CL1. In some embodiments, the modulator is an interleukin, e.g., IL-1 alpha, IL-1 beta, IL-2, IL-3, IL-4, IL-5, IL-6 (BSF-2), IL-7, IL-8, IL-9, IL-10, IL-11, IL-12, IL-13, IL-14, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33 or IL-35.

In some embodiments, a modulator can be a FLT3 inhibitor (e.g., AC220, e.g., at 100 nM; Tandutinib [T] e.g., at 0.5 uM), a DNA damaging agent (e.g., AraC, e.g., at 0.5 μg/ml, 2 um)), A DNMT inhibitor (e.g., zazcitidine, e.g., at 2.5 μM or Decitabine, e.g., at 0.625 μM)), a PARP inhibitor (e.g., AZD2281, e.g., at 5 μM), a PI3K and mTor dual inhibitor (e.g., BEZ235, e.g., at 50 nM), a proteosome inhibitor (e.g., bortezomib at 10 nM or 50 nM), a PI3 Kdelta inhibitor (e.g., CAL-101, e.g., at 0.5 μM), a MEK inhibitor (e.g., AZD6244, e.g., at 1 μM), a DNA synthesis inhibitor (e.g., clofarabine, e.g., at 0.25 μM), a JAK inhibitor (e.g., CP690550(CP)), e.g., at 1 μM; CYT387 e.g., at 1 μM; INCB018424 at 1 μM)), a topoisomerase inhibitor (e.g., etoposide, e.g., at 15 μg/ml), a mTor inhibitor (e.g., Everolimus (RAD0001) e.g., at 10 nM), a PI3K inhibitor (e.g., GDC-0941 [G] e.g., at 1 μM), a BCR-ABL, cKit, or PDGR-R inhibitor (e.g., Imatinib e.g., at 1 μM), an HSP90 inhibitor (e.g., NVP-AUY922 e.g., at 50 nM), a VEGFR, PDGFR, RAF, FLT3, or cKIT inhibitor (e.g., Sorafenib, e.g., at 5 μM), a PDGF-R, VEGF-R, cKIT, FLT3, RET, or CSF-1R inhibitor (e.g., Sunitinib, e.g., at 50 nM), an alkylating agent (e.g., Temozolomide, e.g., at 2 μg/ml (10.3 μM), or an HDAC inhibitor (e.g., Vorinostat (SAHA, Zolinza, e.g., at 2.5 uμM). See Table 1 for additional information on modulators and exemplary concentrations of the modulators.

TABLE 1
Exemplary drugs and concentrations of drugs.

| | Mechanism | |
|---|---|---|
| Drug and | of | |
| concentration | Action | Details |
| AC220 100 nM | FLT3 | AC220 can be used to treat Acute Myeloid Leukemia (AML), a |
| | inhibitor | common type of blood cancer in adults. AC220 can target the kinase |
| | | FLT3, which is mutated and constitutively activated in 25-40 |
| | | percent of AML patients, causing poor prognosis and decreased |
| | | response to existing treatments including chemotherapy and stem |
| | | cell treatments. AC220 can be orally bioavailable and can induce |
| | | tumor regression in a xenograft model at low doses. Ref: |
| | | http://bloodjournal.hematologylibrary.org/content/114/14/2984.full. |
| | | AC220 can be well tolerated and escalated to 450 mg daily on an |
| | | intermittent dosing regimen, and PK has been evaluated up to 300 mg. |
| | | AC220 half-life can be 2.5 days, exhibiting minimal peak and |
| | | trough variation of plasma levels. AC220 plasma exposure in AML |
| | | patients can be sustained between dose intervals and can continue to |
| | | increase in a dose-proportional manner from 12 mg to 300 mg daily, |
| | | with steady-state plasma concentrations achieving greater than 1,500 nM |
| | | at 300 mg. |
| | | Administering a 100 nM concentration of AC220 can block ~80-90% |
| | | of the FLT3 induced pAKT signal. |
| AraC 0.5 μg/ml | DNA | AraC (cytarbine) can be used to treat certain types of leukemia and |
| (2 μM) | damaging | can prevent the spread of leukemia to the meninges (three thin |
| | agent | layers of tissue that cover and protect the brain and spinal cord). |
| | | Cytarabine can acts through direct DNA damage and incorporation |
| | | into DNA. Cytarabine can be cytotoxic to a wide variety of |
| | | proliferating mammalian cells in culture. It can exhibit cell phase |
| | | specificity, primarily killing cells undergoing DNA synthesis (S- |
| | | phase) and under certain conditions can block the progression of |
| | | cells from the G1 phase to the S-phase. Cytarabine can inhibit DNA |
| | | polymerase. A limited, but significant, incorporation of cytarabine |
| | | into both DNA and RNA has also been reported. Cmax = 10 μM after |
| | | 100 ng/m², proportionally higher up to 3 g/m² (2 H inf.) |
| Azacitidine 2.5 μM | DNMT | Cells in the presence of azacitidine incorporate it into DNA during |
| | Inhibitor | replication and RNA during transcription. The incorporation of |
| | | azacitidine into DNA or RNA inhibits methyltransferase thereby |
| | | causing demethylation in that sequence, affecting the way that cell |
| | | regulation proteins are able to bind to the DNA/RNA substrate. |
| | | Inhibition of DNA methylation occurs through the formation of |
| | | stable complexes between the molecule and with DNA |
| | | methyltransferases, thereby saturating cell methylation machinery. |
| | | In vivo: Cmax = 1.42-4.72 μM. |
| AZD2281 | PARP | AZD2281 (Olaparib) can be used to treat breast, ovarian, and |
| 5 μM | inhibitor | prostate cancers caused by mutations in the BRCA1 and BRCA2 |
| | | genes. AZD2281 can be a PARP inhibitor. MTD (maximum |
| | | tolerated dose) can be 400 mg bd, continuously. Cmax (maximum |
| | | plasma concention) can be ~6 ug/ml (13.8uM) at MTD. PD |
| | | (pharmacodynamic) effects can be seen at doses >60 mg. |
| BEZ235 | PI3K and | BEZ235 or NVP-BEZ235 can be an imidazoquinoline derivative |
| 50 nM | mTor dual | and PI3K inhibitor. BEZ235 can inhibit PI3K and mTOR kinase |
| | inhibitor | activity by binding to the ATP-binding cleft of these enzymes. Ref. |
| | | Maira, SM., et al Mol Cancer Ther, 2008, 7(7). Preclinical data |
| | | show that BEZ235 has strong anti-proliferative activity against |
| | | tumour xenografts that have abnormal PI3K signalling, including |
| | | loss of PTEN function or gain-of-function PI3K mutations. |
| | | Pharmcologically active exposure levels can reach doses of 400-1100 mg/ |
| | | day (decreased pS6, CT, PET; ASCO 2010). pAKT and |
| | | pS6 IC50 on H460 cell line can be 10 nM and 50 nM respectively. |
| Bortezomib | Proteosome | Bortezomib can be a drug used to treat multiple myeloma. It can be |
| 10 nM and | inhibitor | used to treat mantle cell lymphoma in patients who have already |
| 50 nM* | | received at least one other type of treatment. Bortezomib can block |
| | | several molecular pathways in a cell and can cause cancer cells to |
| | | die. It can be a type of proteasome inhibitor and a type of dipeptidyl |
| | | boronic acid. Also called PS-341 and velcade. 10 nM blocks |
| | | proteome activity [BLOOD, 16 DECEMBER 2010 VOLUME 116, |
| | | NUMBER 25]. Effect of Bort on the prolifeation of AML cell lines: |
| | | IC90 ~10-50 nM. [haematologica. 2008 Jan; 93(1): 57-66]. In vivo: |
| | | tandard dose of 1.3 mg/m2 twice weekly for 2 wks (day 1-4-8-11), |
| | | with 1 wk rest, for up to 8 cycles. Ave Cmax = 130 ng/ml (338.33 nM). |
| | | Prescribing info says Cmax is 112 ng/ml (291 nM) with a T_{1/2} |
| | | of 76 to 108 hrs. |
| CAL-101 | PI3Kdelta | CAL-101 can be a potent and selective inhibitor of PI3K-δ iso form. |
| 0.5 μM | inhibitor | Nodality IC₅₀ (anti-IgM_pAKT induced PBMC ~10 nM. 40 nM |
| | | blocked ~90%. Ref: Herman, Sarah EM et al. Blood. Jun. 3, 2010 |
| | | prepub online. |
| | | (http://bloodjournal.hematologylibrary.org/content/early/2010/06/03/blood- |
| | | 2010-02-271171.full.pdf+html). |
| | | Increases in Cmax and |
| | | AUC can be less than dose proportional, revealing minimal gains in |
| | | plasma exposure at dose levels >150 mg BID. The mean volume of |
| | | distribution can be moderate at 57.7 L. The t_{1/2} can be ~8 hours |
| | | across all dose levels and there can be no plasma accumulation over |
| | | 7 or 28 days. The collective data support BID dosing at ≧150 mg; |
| | | dose levels in this range maintain steady-state trough plasma |
| | | concentrations that are >10-fold above the EC50 for the in vitro |
| | | whole-blood assay.” 620 nM may be the steady state concentration. |
| AZD6244 1uM | MEK | AZD6244 (ARRY-142886) can be a potent, selective, and ATP |
| | inhibitor | uncompetitive inhibitor of MEK½ kinases. Activating mutations |
| | | in the BRAF gene, e.g., V600E, are associated with poorer |
| | | outcomes in patients with papillary thyroid cancer. MAPK kinase |
| | | (MEK), immediately downstream of BRAF, is a promising target |
| | | for ras-raf-MEK-ERK pathway inhibition. In addition to thyroid |
| | | cancer, BRAF-activating mutations can be prevalent in melanoma (−59%), |
| | | colorectal cancer (5-22%), serous ovarian cancer (−30%), and |
| | | several other tumor types. Davies H et al. Nature. 2002 Jun |
| | | 27; 417(6892): 949-54 |
| | | At twice daily dosing (75 mg), Cmax can be 1439 ng/ml (3.2 μM) at |
| | | 1 hr post dose. PD effects of ~80% pERK inhibition can be seen at |
| | | ~1000 ng/ml plasma conc. in blood lymphocytes used as a surrogate |
| | | readout (Clin Cancer Res; 16(5) Mar. 1, 2010). At 1 μM in vitro, 85-95% |
| | | of PMA induced pERK can be inhibited (IC₅₀ ~100 nM) in |
| | | lymphocytes from PBMCs. |
| Clofarabine | DNA | Clofarabine (Clolar, Genzyme) has been studied in the treatment of |
| 0.25 μM | synthesis | various types of leukemia and is FDA approved for the treatment of |
| | inhibitor | childhood acute lymphoblastic leukemia. It is structurally related to |
| | | fludarabine and cladribine, sharing some characteristics and |
| | | avoiding others. Clofarabine can exert its antineoplastic activity |
| | | through several mechanisms. The active metabolite of clofarabine |
| | | can be its triphosphate form. This molecule can compete with |
| | | deoxyadenosine triphosphate for the ribonucleotide reductase and |
| | | DNA polymerase, which can lead to decreased DNA synthesis and |
| | | repair, inhibit DNA strand elongation and cell replication. |
| | | Pretreatment with clofarabine before cytarabine administration can |
| | | lead to increases in intracellular concentrations of cytarabine |
| | | triphosphate, the active form of cytarabine. The standard dose of |
| | | clofarabine can be 52 mg/m2 for pediatrics and 40 mg/m2 in adults |
| | | which leads to an accumulation of plasma clofarabine of 0.5 to |
| | | 3 μM. (Clin Cancer Res 2003; 9: 6335-6342) |
| CP690550 [CP] | JAKs | CP690550 can be a JAK3 inhibitor. The somatic activating janus |
| 1 μM | | kinase 2 mutation (JAK2)V617F can be detectable in most patients |
| | | with polycythemia vera (PV). Enzymatic assays indicate that both |
| | | JAK1 and JAK2 are 100- and 20-fold less sensitive to inhibition by |
| | | CP-690,550, respectively, when compared with JAK3. |
| | | JAK2V617F-bearing cells were almost 10-fold more sensitive to |
| | | CP-690,550 compared with JAK2WT cells, with IC₅₀s of 0.25 μM |
| | | and 2.11 μM, respectively. In vivo: 30 mg BID. Cmax = 364.39 ng/ml |
| | | (1.16 uM), T½ 2.6 hrs, (Br J Clin Pharmacol/69:2/143-151/ |
| | | 143). GM-CSF_pSTAT5 inhibition can be ~300 nM IC₅₀ |
| | | (JAK2 driven) and ~130 nM for G-CSF (JAK3 driven). |
| CYT387 | JAK | CYT387 can be a JAK inhibitor. Reported activities: (biochemical) |
| 1 μM | inhibitor | JAK2 (18 nM), JAK1(11 nM), JAK3 (155). Ba/F3-wt (+IL-3, |
| | | proliferation) JAK2 wt 1424 nM. PBMCs (monos)/GM- |
| | | CSF/pSTAT5 can have 1109 nM IC₅₀ with IC90 ~333 nM. pAKT |
| | | inhibition (same cells, same stim) can have 129 nM IC₅₀ with |
| | | ~1000 nM IC90. |
| | | http://www.nature.com/leu/journal/v23/n8/pdf/leu200950a.pdf |
| Decitabine | DNMT | Decitabine (Dacogen) is a drug that can be used to treat |
| 0.625 μM | inhibitor | myelodysplastic syndromes. It can be a type of antimetabolite. |
| | | Decitabine is indicated for treatment of patients with |
| | | myelodysplastic syndrome (MDS). Decitabine can exert its |
| | | antineoplastic effects following its conversion to decitabine |
| | | triphosphate, where the drug directly incorporates into DNA and |
| | | inhibits DNA methyltransferase, the enzyme that is responsible for |
| | | methylating newly synthesized DNA in mammalian cells. This can |
| | | result in hypomethylation of DNA and cellular differentiation or |
| | | apoptosis. Decitabine inhibits DNA methylation in vitro, which can |
| | | be achieved at concentrations that do not cause major suppression of |
| | | DNA synthesis. Decitabine-induced hypomethylation in neoplastic |
| | | cells can restore normal function to genes that play a role in the |
| | | control of cellular differentiation and proliferation. In rapidly |
| | | dividing cells, the cytotoxicity of decitabine can also be attributed to |
| | | the formation of covalent adducts between DNA methyltransferase |
| | | and decitabine that has been incorporated into DNA. Non- |
| | | proliferating cells can be relatively insensitive to decitabine. |
| | | Decitabine can be cell cycle specific and can act peripherally in the |
| | | S phase of the cell cycle. In AML cell lines (KG-1, THP-1), |
| | | decitabine can inhibit DNMT1 at 0.1 μM Cmax (IV 15 mg/m2 IV |
| | | over 3 hrs, every 8 hrs, for 3 days) can be 0.3-1.6 μM (Hollenbach |
| | | PW et al. PLoS ONE 5(2): e9001). Decitabine can be used at |
| | | 0.625 μM in vitro 24-48 hrs. |
| Etoposide | topoisomerase | Etoposide (Toposar, Vepesid) can be used to treat testicular and |
| 15 μg/ml | inhibitor | small cell lung cancers. Etoposide can block certain enzymes used |
| | | needed for cell division and DNA repair, and it can kill cancer cells. |
| | | Etoposide is a podophyllotoxin derivative and can inhibit |
| | | topoisomerase. Two different dose-dependent responses can be |
| | | observed with etoposide. At high concentrations (10 μg/mL or |
| | | more), lysis of cells entering mitosis can be observed. At low |
| | | concentrations (0.3 to 10 μg/mL), cells can be inhibited from |
| | | entering prophase. Etoposide can induce DNA strand breaks by an |
| | | interaction with DNA-topoisomerase II or the formation of free |
| | | radicals. In adults with normal renal and hepatic function, an 80 mg/m2 |
| | | IV dose given over 1 hour averaged an etoposide plasma |
| | | Cmax of 14.9 mcg/ml. Following 500 mg/h IV infusions of 400, |
| | | 500, or 600 mg/m2, etoposide plasma peak concentrations of 26 to |
| | | 53, 27 to 73, and 42 to 114 mcg/ml, respectively, can be attained. |
| | | With continuous IV infusion of 100 mg/m2 daily for 72 hours, |
| | | plasma drug concentrations of 2 to 5 mcg/ml can be reached 2 to 3 |
| | | hours after the start of infusion and can be maintained until the end |
| | | of infusion. In children 3 months to 16 years of age with normal |
| | | renal and hepatic function, IV infusions of 200 to 250 mg/m2 given |
| | | over 0.5 to 2.25 hours can result in peak serum etoposide |
| | | concentrations ranging from 17 to 88 mcg/ml. |
| Everolimus | mTor | Everolimus (also known as RAD001) can bind and create a complex |
| (RAD001) | inhibitor | with FKBP12 and can interact with mTor to inhibit downstream |
| 10 nM | | signaling events. In vivo dosing can be either 10 mg/d or 50 mg/wk |
| | | [O'Donnell et al, JCO, 26, (10) Apr. 1, 2008]. At 10 mg/d the Cmax |
| | | can be 61 ng/ml (63 nM) and the trough can be 17 ng/ml (17.7 nM). |
| | | At 50 mg/wk the trough can be 1 ng/ml (~1 nM). [J Clin Oncol |
| | | 26: 1603-1610. 2008]. A 10 nM dose in vitro can inhibit p-S6 |
| | | completely as well as inhibit proliferation of mantle cell line (Jeko) |
| | | [TE Witzig et al, Leukemia (2010), 1-7]. |
| GDC-0941 [G] | PI3K | GDC-0941 can be a PI3K inhibitor. GDC-0941 against p110a can |
| 1 μM | | have an IC₅₀ = 0.003 μM, U87MG; IC₅₀ = 0.95 μM, A2780 I IC₅₀ = 0.14 μM, |
| | | and in vitro metabolic stability in mouse and human can be |
| | | 91.96%. The inhibitions of U87MG, PC3, MDA-MB-361 cancer |
| | | cell proliferation can be (IC50) 0.95, 0.28, and 0.72. GDC-0941 can |
| | | display dose-proportional increases in mean Cmax and AUCinf. |
| | | Decreases in pS6 staining of >50% can occur in paired tumor |
| | | biopsies in addition to decreases of >90% in pAKT levels assayed in |
| | | PRP from patientss treated at 80 mg and higher. Signs of biologic |
| | | activity can be observed in 3 patientss (ovarian cancer, triple |
| | | negative breast cancer, and ocular melanoma) treated at ≧100 mg |
| | | GDC-0941 with reductions (≧30% in mean SUVmax) in tumor FDG |
| | | avidity observed on PET scan and an ~80% decrease in CA-125 in |
| | | an ovarian cancer patient, who remained on-study for ~5 months. |
| | | Conclusions: GDC-0941 can be generally well tolerated at 15 to 130 mg |
| | | QD. Decreases in pAKT levels in PRP and decreases in pS6 |
| | | staining in paired tumor biopsies are consistent with downstream |
| | | modulation of the PI3K pathway. |
| Imatinib | BCR- | Imatinib (Gleevec or STI571) can be used to treat different types of |
| 1 μM | ABL, | leukemia and other cancers of the blood, gastrointestinal stromal |
| | cKit, | tumors, skin tumors called dermatofibrosarcoma protuberans, and a |
| | PDGF-R | rare condition called systemic mastocytosis. Imatinib mesylate can |
| | | block the protein made by the bcr/abl oncogene. It is a type of |
| | | tyrosine kinase inhibitor. The plasma trough level of imatinib at |
| | | steady state can be slightly higher in females than males (1078 [1] |
| | | 515 ng/mL vs 921 531 ng/mL, respectively). (BLOOD, 15 APRIL |
| | | 2008_VOLUME 111, NUMBER 8). Assume trough of 1000 ng/ml = |
| | | 2 μM. |
| INCB018424 | JAK | INCB018424 phosphate can be a potent inhibitor of JAK enzymes |
| 1 μM | inhibitor | with selectivity for JAK1&2, and can be used for the treatment of |
| | | myelofibrosis (MF). In vivo, 25 mg bid and 100 mg qd can be the |
| | | maximum tolerated doses in healthy volunteers. INCB018424 |
| | | dosing: 25 mg bid and 100 mg qd can be the maximum tolerated |
| | | doses in healthy volunteers. At 100 mg 24 h: Cmax 4780 nM; |
| | | Tmax = 1.5 hrs; T½ = 2.8 hrs. The plasma conc. was ~1000 nM at 6 hrs |
| | | post-dose. (Shi et al. J Clin Pharmacol, published online 21 Jan |
| | | 2011.) 1000 nM can completely inhibit GM-CSF_pSTAT5 |
| | | (IC₅₀ = 215 nM). |
| NVP-AUY922 | HSP90 | HSP90 can be a ubiquitously expressed molecular chaperone that |
| 50 nM | | can play a role in the post-translational conformational maturation |
| | | and activation of a large number of client proteins that have been |
| | | implicated in oncogenesis. Inhibition of the ATPase activity at the |
| | | N-terminus of HSP90 is being exploited by all inhibitors that have |
| | | entered the clinic so far. In competitive fluorescence polarization |
| | | assays, NVP-AUY922 inhibited HSP90α and HSP90β with similar |
| | | IC₅₀ (median inhibition concentration) values of 13 and 21 nM |
| | | respectively. In a representative panel of human tumor cell lines |
| | | (including prostate, breast, ovarian, colon, lung, melanoma, and |
| | | glioblastoma), NVP-AUY922 can inhibit cell proliferation with low |
| | | nanomolar potency; GI50 (the concentration that inhibits cell growth |
| | | by 50%) values can be in the range of 2.3 to 50 nM. |
| Sorafenib 5 μM | VEGFR, | Sorafenib can be used to treat advanced kidney cancer and a type of |
| | PDGFR, | liver cancer that cannot be removed by surgery. Sorafenib tosylate |
| | RAF, | can stop cells from dividing and can prevent the growth of new |
| | FLT3, | blood vessels that tumors need to grow. It can inhibit kinases and act |
| | cKIT | as an antiangiogenesis agent. Sorafenib can also be called BAY 43- |
| | | 9006, or Nexavar. Steady state C trough level can be 3 mg/ml at |
| | | 400 mg BID which equals 6.4 μM. |
| Sunitinib 50 nM | PDGF-R, | Sunitinib can be used to treat gastrointestinal stromal tumors (GIST) |
| | VEGF-R, | that have not responded to treatment with imatinib mesylate |
| | cKit, | (Gleevec). Sunitinib can also used to treat advanced kidney cancer. |
| | FLT3, | It can be a type of tyrosine kinase inhibitor, a type of vascular |
| | RET, | endothelial growth factor (VEGF) receptor inhibitor, and a type of |
| | CSF-1R | angiogenesis inhibitor. It can be called SU011248, SU11248, |
| | | sunitinib malate, and Sutent. T max can be between 6 and 12 h. |
| | | With repeat daily dosing, sunitinib can accumulate 3- to 4-fold, and |
| | | the primary active metabolite can accumulate 7- to 10-fold. Steady- |
| | | state concentrations of the primary drug and primary metabolite can |
| | | be achieved within 10 to 14 days. The combined plasma levels of |
| | | sunitinib plus active metabolite can range from 62.9 to 101 ng/mL |
| | | (125.5 nM to 253.4 nM). |
| Tandutinib [T] | FLT3 | Tandutinib (CT53518 and MLN518) can stop cancer cell growth by |
| 0.5 μM | inhibitor | blocking certain enzymes and can also prevent the growth of new |
| | | blood vessels that tumors need to grow. Tandutinib can inhibit |
| | | tyrosine kinases and can act as an antiangiogenesis agent. |
| | | Tandutinib can be given orally in doses ranging from 50 mg to 700 mg |
| | | twice daily The principal dose-limiting toxicity (DLT) of |
| | | tandutinib can be reversible generalized muscular weakness, fatigue, |
| | | or both, occurring at doses of 525 mg and 700 mg twice daily. |
| | | Tandutinib's pharmacokinetics can be characterized by slow |
| | | elimination, with achievement of steady-state plasma concentrations |
| | | requiring greater than 1 week of dosing. Tandutinib can inhibit |
| | | phosphorylation of FLT3 in circulating leukemic blasts. Eight |
| | | patients had FLT3-ITD mutations; 5 of these were evaluable for |
| | | assessment of tandutinib's antileukemic effect. Two of the 5 |
| | | patients, treated at 525 mg and 700 mg twice daily, showed |
| | | evidence of antileukemic activity, with decreases in both peripheral |
| | | and bone marrow blasts. (Blood. 2006 December 1; 108(12): 3674-3681). |
| | | At this dose a mean plasma concentration can be ~300 ng/ml |
| | | (533 nM). |
| Temozolomide | alkylating | Temozolomide (TMZ) is an imidazotetrazine derivative of the |
| 2 μg/ml (10.3 μM) | agent | alkylating agent dacarbazine. It can undergo rapid chemical |
| | | conversion in the systemic circulation at physiological pH to the |
| | | active compound, MTIC (monomethyl triazeno imidazole |
| | | carboxamide). Temozolomide can exhibit schedule-dependent |
| | | antineoplastic activity by interfering with DNA replication. |
| | | Temozolomide can demonstrate activity against recurrent glioma. In |
| | | a recent randomized trial, concomitant and adjuvant temozolomide |
| | | chemotherapy with radiation significantly can improve progression |
| | | free survival and overall survival in glioblastoma multiforme |
| | | patients. Adult MTD = 200 ng/m2/day (Seiter K et al. J Clin Onco |
| | | 20: 3249-3253, 2002). 200 ng/m2/day = 9.3 ug/ml(47.9 μM). The |
| | | T½ is 100 min. Two μg/ml (10.3 μM) is well below the Cmax. |
| Vorinostat | HDAC | Vorinostat (SAHA) is a synthetic hydroxamic acid derivative that |
| (SAHA, | inhibitor | can have antineoplastic activity. Vorinostat, a second generation |
| Zolinza) 2.5 μM | | polar-planar compound, can bind to the catalytic domain of the |
| | | histone deacetylases (HDACs). This can allow the hydroxamic |
| | | moiety to chelate zinc ion located in the catalytic pockets of HDAC, |
| | | thereby inhibiting deacetylation and leading to an accumulation of |
| | | both hyperacetylated histones and transcription factors. |
| | | Hyperacetylation of histone proteins can result in the upregulation |
| | | of the cyclin-dependent kinase p21, followed by G1 arrest. |
| | | Hyperacetylation of non-histone proteins such as tumor suppressor |
| | | p53, alpha tubulin, and heat-shock protein 90 can produce additional |
| | | anti-proliferative effects. This agent can also induce apoptosis and |
| | | sensitize tumor cells to cell death processes. SAHA can be used at |
| | | 2.5 μM (0.66 μg/ml). Cmax can be1.81 +/− .70 μM [1.11-2.51 μM]. |
| | | A concentration of 2.5 μM is within the Cmax and is also near the |
| | | reported ED50 reported for AML cells lines (Hollenbach PW et al. |
| | | PLoS ONE 5(2): e9001) |

### Activatable Elements

The methods and compositions described herein may be employed to examine and profile the status of any activatable element in a cellular pathway, or collections of such activatable elements. Single or multiple distinct pathways may be profiled (sequentially or simultaneously), or subsets of activatable elements within a single pathway or across multiple pathways may be examined (again, sequentially or simultaneously).

Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element can be measured through the use of a binding element that recognizes a specific activation state, only those activatable elements in the specific activation state recognized by the binding element, representing some fraction of the total number of activatable elements, can be bound by the binding element to generate a measurable signal. The measurable signal corresponding to the summation of individual activatable elements of a particular type that are activated in a single cell can be the “activation level” for that activatable element in that cell. The activation state of an individual activatable element can be represented as continuous numeric values representing a quantity of the activatable element or can be discretized into categorical variables. For instance, the activation state may be discretized into a binary value indicating that the activatable element is either in the on or off state. As an illustrative example, and without intending to be limited to any theory, an individual phosphorylatable site on a protein can be phosphorylated and then be in the “on” state or it can not be phosphorylated and hence, it will be in the “off’state. See Blume-Jensen and Hunter, Nature, vol 411, 17 May 2001, p 355-365. The terms “on” and “off,” when applied to an activatable element that is a part of a cellular constituent, can be used here to describe the state of the activatable element (e.g., phosphorylated is “on” and non-phosphorylated is “off’), and not the overall state of the cellular constituent of which it is a part.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis for determining the activation levels of one or more activatable elements in cells may use the distribution of activation levels for one or more specific activatable elements which will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to determine the physiological status of a cell in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may not contain activatable elements, of one or more cells in a population of cells may be used to determine the physiological status of the cell population.

In some embodiments, the basis for determining the physiological status of a population of cells may use the position of a cell in a contour or density plot of the distribution of the activation levels. The contour or density plot represents the number of cells that share a characteristic such as the activation level of activatable proteins in response to a modulator. For example, when referring to activation levels of activatable elements in response to one or more modulators, normal individuals and patients with a condition might show populations with increased activation levels in response to the one or more modulators. However, the number of cells that have a specific activation level (e.g., a specific amount of an activatable element) might be different between cells from normal individuals and cells from patients with a condition. Thus, the physiological status of a cell can be determined according to its location within a given region in the contour or density plot.

In a specific embodiment, methods may be used to represent the distribution of the activation levels as a one-dimensional vector of values. For additional information, see e.g., PCT Publication No. WO/2007/117423.

In another specific embodiments, methods may be used to model the data within the homogeneous population of cells. These methods may incorporate state transition modeling as outlines. Bayesian network, belief network or directed acyclic graphical model can be a probabilistic graphical model that can represent a set of random variables and their conditional dependencies via a directed acyclic graph (DAG). For example, a Bayesian network can represent the probabilistic relationships between diseases and symptoms. Given symptoms, the network can be used to compute the probabilities of the presence of various diseases. For additional information, see e.g., U.S. Patent Application No. 20070009923.

In addition to activation levels of intracellular activatable elements, expression levels of intracellular or extracellular biomolecules, e.g., proteins, may be used alone or in combination with activation states of activatable elements to determine the physiological status of a population of cells. Further, additional cellular elements, e.g., biomolecules or molecular complexes such as RNA, DNA, carbohydrates, metabolites, and the like, may be used in conjunction with activatable states, expression levels or any combination of activatable states and expression levels in the determination of the physiological status of a population of cells encompassed here.

In some embodiments, other characteristics that affect the status of a cellular constituent may also be used to determine the physiological status of a cell. Examples include the translocation of biomolecules or changes in their turnover rates and the formation and disassociation of complexes of a biomolecule. Such complexes can include multi-protein complexes, multi-lipid complexes, homo- or hetero-dimers or oligomers, and combinations thereof. Other characteristics include proteolytic cleavage, e.g., from exposure of a cell to an extracellular protease or from the intracellular proteolytic cleavage of a biomolecule.

Additional elements may also be used to determine the physiological status of a cell, such as the expression level of extracellular or intracellular markers, nuclear antigens, enzymatic activity, protein expression and localization, cell cycle analysis, chromosomal analysis, teleomere length analysis, telomerase activity, cell volume, and morphological characteristics like granularity and size of nucleus or other distinguishing characteristics. For example, myeloid lineage cells can be further subdivided based on the expression of cell surface markers such as CD14, CD15, or CD33, CD34 and CD45.

Alternatively, different homogeneous populations of cells can be aggregated based upon shared characteristics that may include inclusion in one or more additional cell populations or the presence of extracellular or intracellular markers, similar gene expression profile, nuclear antigens, enzymatic activity, protein expression and localization, cell cycle analysis, chromosomal analysis, cell volume, teleomere length analysis, telomerase activity, and morphological characteristics like granularity and size of nucleus or other distinguishing characteristics.

In some embodiments, the physiological status of one or more cells is determined by examining and profiling the activation level of one or more activatable elements in a cellular pathway. In some embodiments, the activation levels of one or more activatable elements of a cell from a first population of cells and the activation levels of one or more activatable elements of a cell from a second population of cells are correlated with a condition. In some embodiments, the first and second homogeneous populations of cells are hematopoietic cell populations. In some embodiments, the activation levels of one or more activatable elements of a cell from a first population of hematopoietic cells and the activation levels of one or more activatable elements of cell from a second population of hematopoietic cells are correlated with a neoplastic, autoimmune or hematopoietic condition as described herein. Examples of different populations of hematopoietic cells include, but are not limited to, pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells.

In some embodiments, the activation level of one or more activatable elements in single cells in the sample is determined. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. The term “plurality” as used herein refers to two or more. In some embodiments, at least about 2, 3, 4, 5, 6, 7, 8, 9, 10, or more than 10 intracellular activatable elements are determined. In some embodiments, about 1-10, 1-7, 1-5, 2-10, 2-7, or 2-5 intracellular activatable elements are determined.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

In some embodiments, the activatable element is a protein. Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 61/048,886, 61/048,920 and Shulz et al, Current Protocols in Immunology 2007, 7:8.17.1-20.

In some embodiments, the protein that may be activated is selected from the group consisting of HER receptors, PDGF receptors, FLT3 receptor, Kit receptor, FGF receptors, Eph receptors, Trk receptors, IGF receptors, Insulin receptor, Met receptor, Ret, VEGF receptors, erythropoetin receptor, thromobopoetin receptor, CD114, CD116, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3a, GSK3β, Cdks, CLKs, PKR, PI3-Kinase class 1, class 2, class 3, mTor, SAPK/JNK1, 2, 3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1, 2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FM transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, Ets-1, Ets-2, SPDEF, GABPα, Tel, Tel2, WT-1, HMGA, pS6, 4EPB-1, eIF4E-binding protein, RNA polymerase, initiation factors, elongation factors.

In some embodiments, the methods described herein are employed to determine the activation level of an activatable element, e.g., in a cellular pathway. Methods and compositions are provided for the determination of the physiological status of a cell according to the activation level of an activatable element in a cellular pathway. Methods and compositions are provided for the determination of the physiological status of a cell in a first cell population and a cell in a second cell population according to the activation level of an activatable element in a cellular pathway in each cell. The cells can be a hematopoietic cell and examples are provided herein.

In some embodiments, the determination of the physiological status of cells in different populations according to activation level of an activatable element, e.g., in a cellular pathway comprises classifying at least one of the cells as a cell that is correlated with a clinical outcome. Examples of clinical outcomes, staging, as well as patient responses are provided herein.

### Signaling Pathways

In some embodiments, the methods described herein are employed to determine the activation level of an activatable element in a signaling pathway. In some embodiments, the physiological status of a cell is determined, as described herein, according to the activation level of one or more activatable elements in one or more signaling pathways. Signaling pathways and their members have been extensively described. See (Hunter T. Cell Jan. 7, 2000; 100(1): 13-27; Weinberg, 2007; and Blume-Jensen and Hunter, Nature, vol 411, 17 May 2001, p 355-365 cited above). Exemplary signaling pathways include the following pathways and their members: the JAK-STAT pathway including JAKs, STATs 2, 3 4 and 5, the FLT3L signaling pathway, the MAP kinase pathway including Ras, Raf, MEK, ERK and Elk; the PI3K/Akt pathway including PI-3-kinase, PDK1, Akt and Bad; the NF-κB pathway including IKKs, IkB and NF-κB and the Wnt pathway including frizzled receptors, beta-catenin, APC and other co-factors and TCF (see Cell Signaling Technology, Inc. 2002 Catalog pages 231-279 and Hunter T., supra.). In some embodiments, the correlated activatable elements being assayed (or the signaling proteins being examined) are members of the MAP kinase, Akt, NFkB, WNT, STAT and/or PKC signaling pathways.

In some embodiments, methods are employed to determine the activation level of a signaling protein in a signaling pathway known in the art including those described herein. Exemplary types of signaling proteins include, but are not limited to, kinases, kinase substrates (i.e., phosphorylated substrates), phosphatases, phosphatase substrates, binding proteins (such as 14-3-3), receptor ligands and receptors (cell surface receptor tyrosine kinases and nuclear receptors)). Kinases and protein binding domains, for example, have been well described (see, e.g., Cell Signaling Technology, Inc., 2002 Catalogue “The Human Protein Kinases” and “Protein Interaction Domains” pgs. 254-279).

Exemplary signaling proteins include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks (e.g., Erk1, Erk2), IKKs, GSK3a, GSK3β, Cdks, CLKs, PKR, PI3-Kinase class 1, class 2, class 3, mTor, SAPK/JNK1, 2, 3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1, 2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, and elongation factors.

In some embodiments the protein is selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIM, SHIP2, sSHIP, PTEN, Shc, Grb2, PDK1, SGK, Akt1, Akt2, Akt3, TSC1, 2, Rheb, mTor, 4EBP-1, p70S6Kinase, S6, LKB-1, AMPK, PFK, Acetyl-CoAa Carboxylase, DokS, Rafs, Mos, Tpl2, MEK1/2, MLK3, TAK, DLK, MKK3/6, MEKK1, 4, MLK3, ASK1, MKK4/7, SAPK/JNK1, 2, 3, p38s, Erk1/2, Syk, Btk, BLNK, LAT, ZAP70, Lck, Cbl, SLP-76, PLCyi, PLCy 2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H₂B, HATs, HDACs, PKR, Rb, Cyclin D, Cyclin E, Cyclin A, Cyclin B, P16, p14Arf, p27KIP, p21C1P, Cdk4, Cdk6, Cdk7, Cdk1, Cdk2, Cdk9, Cdc25, A/B/C, Abl, E2F, FADD, TRADD, TRAF2, RIP, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(Rel A), IKKα, PKA, PKCα, PKC β, PKCθ, PKCδ, CAMK, Elk, AFT, Myc, Egr-1, NFAT, ATF-2, Mdm2, p53, DNA-PK, Chk1, Chk2, ATM, ATR, βcatenin, CrkL, GSK3α, GSK3β, and FOXO.

In some embodiments, the methods described herein are employed to determine the activation level of an activatable element in a signaling pathway. See U.S. Ser. Nos. 61/048,886 and 61/048,920 which are incorporated by reference. Methods and compositions are provided for the determination of a physiological status of a cell according to the status of an activatable element in a signaling pathway. Methods and compositions are provided for the determination of a physiological status of cells in different populations of cells according to the status of an activatable element in a signaling pathway. The cells can be hematopoietic cells. Examples of hematopoietic cells are provided herein.

In some embodiments, the determination of a physiological status of cells in different populations of cells according to the activation level of an activatable element in a signaling pathway comprises classifying the cell populations as cells that are correlated with a clinical outcome. Examples of clinical outcome, staging, patient responses and classifications are provided herein.

### Binding Element

In some embodiments, the activation level of an activatable element is determined. In one embodiment, the determination is made by contacting a cell from a cell population with a binding element that is specific for an activation state of the activatable element. The term “binding element” can include any molecule, e.g., peptide, nucleic acid, small organic molecule which is capable of detecting an activation state of an activatable element over another activation state of the activatable element. Binding elements and labels for binding elements are shown in U.S. Ser. No. 61/048,886; 61/048,920 and 61/048,657.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein can include both naturally occurring and synthetic amino acids. For example, homo-phenylalanine, citrulline and noreleucine are considered amino acids. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. 5218: U138 Part 2 Aug. 22, 1999, both of which are expressly incorporated by reference herein.

Methods described herein may be used to detect any particular activatable element in a sample that is antigenically detectable and antigenically distinguishable from another activatable element which is present in the sample. For example, the activation state-specific antibodies can be used in the present methods to identify distinct signaling cascades of a subset or subpopulation of complex cell populations; and/or the ordering of protein activation (e.g., kinase activation) in potential signaling hierarchies. Hence, in some embodiments the expression and phosphorylation of one or more polypeptides are detected and quantified using methods described herein. In some embodiments, the expression and phosphorylation of one or more polypeptides that are cellular components of a cellular pathway are detected and quantified using methods described herein. As used herein, the term “activation state-specific antibody” or “activation state antibody” or grammatical equivalents thereof, can refer to an antibody that specifically binds to a corresponding and specific antigen. The corresponding and specific antigen can be a specific form of an activatable element. The binding of the activation state-specific antibody can be indicative of a specific activation state of a specific activatable element.

In some embodiments, the binding element is an antibody. In some embodiments, the binding element is an activation state-specific antibody.

The term “antibody” can include full length antibodies and antibody fragments, and may refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory. They can be humanized, glycosylated, bound to solid supports, or posses other variations. See U.S. Ser. Nos. 61/048,886, 61/048,920, and 61/048,657 for more information about antibodies as binding elements.

Activation state specific antibodies can be used to detect kinase activity. Additional means for determining kinase activation are provided herein. For example, substrates that are specifically recognized by protein kinases and phosphorylated thereby are known. Antibodies that specifically bind to such phosphorylated substrates but do not bind to such non-phosphorylated substrates (phospho-substrate antibodies) may be used to determine the presence of activated kinase in a sample.

The antigenicity of an activated isoform of an activatable element can be distinguishable from the antigenicity of non-activated isoform of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of a moiety to an element, such as a phosphate moiety, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are centered around the active site of the element, although as is known in the art, conformational changes in one area of an element may cause alterations in different areas of the element as well.

Many antibodies, many of which are commercially available (for example, see Cell Signaling Technology, www.cellsignal.com or Becton Dickinson, www.bd.com) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, FLT3 receptor, Kit receptor, FGF receptors, Eph receptors, Trk receptors, IGF receptors, Insulin receptor, Met receptor, Ret, VEGF receptors, TIE1, TIE2, erythropoetin receptor, thromobopoetin receptor, CD114, CD116, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, GSK3a, GSK3β, Cdks, CLKs, PKR, PI3-Kinase class 1, class 2, class 3, mTor, SAPK/JNK1, 2, 3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PPS, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1, 2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets family transcription factors, Ets-1, Ets-2, Tel, Tel2, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-FOXO STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, elongation factors. In some embodiments, the protein is S6.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope may be used to produce such an antibody fragment by recombinant means well known in the art.

In some embodiments, aromatic amino acids of protein binding elements may be replaced with other molecules. See U.S. S. Nos. 61/048,886, 61/048,920, and 61/048,657.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol. (2001) 31:3535-45; Cochran et al. J. Am. Chem. Soc. (2001) 123:625-32; Houimel et al. Int. J. Cancer (2001) 92:748-55, each incorporated herein by reference). Further, fluorophores can be attached to such antibodies for use in the methods described herein.

A variety of recognitions structures are known in the art (e.g., Cochran et al., J. Am. Chem. Soc. (2001) 123:625-32; Boer et al., Blood (2002) 100:467-73, each expressly incorporated herein by reference) and can be produced using methods known in the art (see e.g., Boer et al., Blood (2002) 100:467-73; Gualillo et al., Mol. Cell Endocrinol. (2002) 190:83-9, each expressly incorporated herein by reference), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In some embodiments, the recognition structure is an anti-laminin single-chain antibody fragment (scFv) (see e.g., Sanz et al., Gene Therapy (2002) 9:1049-53; Tse et al., J. Mol. Biol. (2002) 317:85-94, each expressly incorporated herein by reference).

In some embodiments the binding element is a nucleic acid. The term “nucleic acid” include nucleic acid analogs, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

In some embodiment the binding element is a small organic compound. Binding elements can be synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or binding elements that can then be used in the methods and compositions described herein.

In some embodiments the binding element is a carbohydrate. As used herein the term carbohydrate can include any compound with the general formula (CH₂0)ₙ. Examples of carbohydrates are mono-, di-, tri- and oligosaccharides, as well polysaccharides such as glycogen, cellulose, and starches.

In some embodiments the binding element is a lipid. As used herein the term lipid can include any water insoluble organic molecule that is soluble in nonpolar organic solvents. Examples of lipids are steroids, such as cholesterol, phospholipids such as sphingomeylin, and fatty acyls, glycerolipids, glycerophospholipids, sphingolipids, saccharolipids, and polyketides, including tri-, di- and monoglycerides and phospholipids. The lipid can be a hydrophobic molecule or amphiphilic molecule.

Examples of activatable elements, activation states and methods of determining the activation level of activatable elements are described in US publication number 20060073474 entitled “Methods and compositions for detecting the activation state of multiple proteins in single cells” and US publication number 20050112700 entitled “Methods and compositions for risk stratification” the content of which are incorporate here by reference.

### Labels

The methods and compositions provided herein provide binding elements comprising a label or tag. By label is meant a molecule that can be directly (i.e., a primary label) or indirectly (i.e., a secondary label) detected; for example a label can be visualized and/or measured or otherwise identified so that its presence or absence can be known. Binding elements and labels for binding elements are shown in U.S. Ser. No. 61/048,886, 61/048,920, and 61/048,657.

A compound can be directly or indirectly conjugated to a label which provides a detectable signal, e.g., radioisotopes, fluorescers, enzymes, antibodies, particles such as magnetic particles, chemiluminescers, molecules that can be detected by mass spec, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes. In some embodiments, these labels may be conjugated to the binding elements.

In some embodiments, one or more binding elements are uniquely labeled. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels can fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.) and magnetic particles. In some embodiments, the detection label is a primary label. A primary label is one that can be directly detected, such as a fluorophore.

Labels include optical labels such as fluorescent dyes or moieties. Fluorophores can be either “small molecule” fluors, or proteinaceous fluors (e.g., green fluorescent proteins and all variants thereof).

In some embodiments, activation state-specific antibodies are labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through Invitrogen, http://probes.invitrogen.com/products/qdot/.

Quantum dot labeled antibodies can be used alone or they can be employed in conjunction with organic fluorochrome-conjugated antibodies to increase the total number of labels available. As the number of labeled antibodies increase so does the ability for subtyping known cell populations. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018,850, entitled Salicylamide-Lanthanide Complexes for Use as Luminescent Markers. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy.

In some embodiments, the activatable elements are labeled with tags suitable for Inductively Coupled Plasma Mass Spectrometer (ICP-MS) as disclosed in Tanner et al. Spectrochimica Acta Part B: Atomic Spectroscopy, 2007 March; 62(3):188-195.

Detection systems based on FRET, discussed in detail below, may be used. FRET can be used in the methods described herein, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

The methods and compositions described herein may also make use of label enzymes. By label enzyme is meant an enzyme that may be reacted in the presence of a label enzyme substrate that produces a detectable product. Suitable label enzymes include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By radioisotope is meant any radioactive molecule. Suitable radioisotopes include, but are not limited to ¹⁴C, ³H, ³²P, ³³P, ³⁵S, ¹²⁵I and ¹³¹I. The use of radioisotopes as labels is well known in the art.

As mentioned, labels may be indirectly detected, that is, the tag is a partner of a binding pair. By “partner of a binding pair” is meant one of a first and a second moiety, wherein the first and the second moiety have a specific binding affinity for each other. Suitable binding pairs include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255: 192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto. Binding pair partners may be used in applications other than for labeling, as is described herein.

A partner of one binding pair may also be a partner of another binding pair. For example, an antigen (first moiety) may bind to a first antibody (second moiety) that may, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair may comprise a label, as described above. It will further be appreciated that this allows for a tag to be indirectly labeled upon the binding of a binding partner comprising a label. Attaching a label to a tag that is a partner of a binding pair, as just described, is referred to herein as “indirect labeling”.

By “surface substrate binding molecule” or “attachment tag” and grammatical equivalents thereof can be meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. In general, surface binding substrate molecules include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

### Detection

In practicing the methods described herein, the detection of the status of the one or more activatable elements can be carried out by a person, such as a technician in the laboratory. The detection of the status of the one or more activatable elements can be carried out using automated systems. In either case, the detection of the status of the one or more activatable elements for use according to the methods described herein can be performed according to standard techniques and protocols well-established in the art.

One or more activatable elements can be detected and/or quantified by any method that detects and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, Far Western, Northern Blot, Southern blot, whole cell staining, immunoelectronmicroscopy, nucleic acid amplification, gene array, protein array, mass spectrometry, nucleic acid sequencing, next generation sequencing, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See U.S. patent application Ser. No. 10/898,734 and Shulz et al., Current Protocols in Immunology, 2007, 78:8.17.1-20 which are incorporated by reference in their entireties.

In some embodiments, methods are provided for determining the activation level on an activatable element for a single cell. The methods may comprise analyzing cells by flow cytometry on the basis of the activation level of at least two activatable elements. Binding elements (e.g., activation state-specific antibodies) can be used to analyze cells on the basis of activatable element activation level, and can be detected as described below. Non-binding element systems as described above can be used in any system described herein.

When using fluorescent labeled components in the methods and compositions described herein, different types of fluorescent monitoring systems, e.g., cytometric measurement device systems, can be used. In some embodiments, flow cytometric systems are used or systems dedicated to high throughput screening, e.g., 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g., size, granularity, or fluorescent intensity.

The detecting, sorting, or isolating step of the methods described herein can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods described herein (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g., a FACSVantage™ Cell Sorter, Becton Dickinson Immunocytometry Systems, San Jose, Calif.) is used to sort and collect cells that may be used as a modulator or as a population of reference cells. In some embodiments, the modulator or reference cells are first contacted with fluorescent-labeled binding elements (e.g., antibodies) directed against specific elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety.

In another embodiment, positive cells can be sorted using magnetic separation of cells based on the presence of an isoform of an activatable element. In such separation techniques, cells to be positively selected can be first contacted with a specific binding element (e.g., an antibody or reagent that binds an isoform of an activatable element). The cells can then be contacted with retrievable particles (e.g., magnetically responsive particles) that can be coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic filed while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods can comprise providing a population of cells and analyzing the population of cells by flow cytometry. Cells can be analyzed on the basis of the activation level of at least one activatable element. In some embodiments, cells are analyzed on the basis of the activation level of at least two activatable elements.

In some embodiments, a multiplicity of activatable element activation-state antibodies are used to simultaneously determine the activation level of a multiplicity of elements.

In some embodiments, cell analysis by flow cytometry on the basis of the activation level of at least two elements is combined with a determination of other flow cytometry readable outputs, such as the presence of surface markers, granularity and cell size to provide a correlation between the activation level of a multiplicity of elements and other cell qualities measurable by flow cytometry for single cells.

The ordering of element clustering events in signal transduction is also provided. For example, an element clustering and activation hierarchy can be constructed based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

As will be appreciated, these methods provide for the identification of distinct signaling cascades for both artificial and stimulatory conditions in cell populations, such as peripheral blood mononuclear cells, or naive and memory lymphocytes.

Cells can be dispersed into a single cell suspension, e.g., by enzymatic digestion with a suitable protease, e.g., collagenase, dispase, etc; and the like. An appropriate solution can be used for dispersion or suspension. Such solution will generally be a balanced salt solution, e.g., normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be fixed, e.g., with 3% paraformaldehyde, and can be permeabilized, e.g., with ice cold methanol; HEPES-buffered PBS containing 0.1% saponin, 3% BSA; covering for 2 min in acetone at −200° C.; and the like as known in the art and according to the methods described herein.

In some embodiments, one or more cells are contained in a well of a 96 well plate or other commercially available multiwell plate. In some embodiments, the reaction mixture or cells are in a cytometric measurement device. Other multiwell plates useful include, but are not limited to 384 well plates and 1536 well plates. Still other vessels for containing the reaction mixture or cells will be apparent to the skilled artisan.

The addition of the components of the assay for detecting the activation level or activity of an activatable element, or modulation of such activation level or activity, may be sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element can bind to the activatable element. When the cell is introduced into the ICP, it can be atomized and ionized. The elemental composition of the cell, including the labeled binding element that is bound to the activatable element, can be measured. The presence and intensity of the signals corresponding to the labels on the binding element can indicate the level of the activatable element on that cell (Tanner et al. Spectrochimica Acta Part B: Atomic Spectroscopy, 2007 March; 62(3):188-195.).

The instant methods and compositions can be used in a variety of other assay formats in addition to flow cytometry analysis. For example, a chip analogous to a DNA chip can be used in the methods provided herein. Arrayers and methods for spotting nucleic acids on a chip in a prefigured array are known. In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell. See U.S. Pat. No. 5,744,934.

In some embodiments confocal microscopy can be used to detect activation profiles for individual cells. Confocal microscopy can use serial collection of light from spatially filtered individual specimen points, which can then be electronically processed to render a magnified image of the specimen. The signal processing involved confocal microscopy can have the additional capability of detecting labeled binding elements within single cells; accordingly in this embodiment the cells can be labeled with one or more binding elements. In some embodiments the binding elements used in connection with confocal microscopy are antibodies conjugated to fluorescent labels; however other binding elements, such as other proteins or nucleic acids are also possible.

In some embodiments, the methods and compositions provided herein can be used in conjunction with an “In-Cell Western Assay.” In such an assay, cells can be initially grown in standard tissue culture flasks using standard tissue culture techniques. Once grown to optimum confluency, the growth media can be removed and cells can be washed and trypsinized. The cells can then be counted and volumes sufficient to transfer the appropriate number of cells can be aliquoted into microwell plates (e.g., Nunc™ 96 Microwell™ plates). The individual wells can then be grown to optimum confluency in complete media whereupon the media can be replaced with serum-free media. At this point controls can be untouched, but experimental wells can be incubated with a modulator, e.g., EGF. After incubation with the modulator cells can be fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC. In a further embodiment, the detecting is by mass spectrometry.

These instruments can fit in a sterile laminar flow or fume hood, or can be enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiments, the methods provided herein include the use of liquid handling components. The liquid handling systems can include robotic systems comprising any number of components. In addition, any or all of the steps outlined herein may be automated; thus, for example, the systems may be completely or partially automated.

There are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems. See U.S. Ser. No. 61/048,657 which is incorporated by reference in its entirety.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in the methods described herein.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods provided herein include the use of a plate reader. See U.S. Ser. No. 61/048,657.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation includes a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices described herein. The general interaction between a central processing unit, a memory, input/output devices, and a bus is known in the art. Thus, a variety of different procedures, depending on the experiments to be run, can be stored in the CPU memory. See U.S. Ser. No. 61/048,657 which is incorporated by reference in its entirety.

These robotic fluid handling systems can utilize any number of different reagents, including buffers, reagents, samples, washes, assay components such as label probes, etc.

Any of the steps described herein can be performed by a computer program product that comprises a computer executable logic that is recorded on a computer readable medium. For example, the computer program can execute some or all of the following functions: (i) exposing different population of cells to one or more modulators, (ii) exposing different population of cells to one or more binding elements, (iii) detecting an activation level of one or more activatable elements, (iv) making a diagnosis or prognosis based on the activation level of one or more activatable elements in the different populations, (v) comparing a signaling profile of a normal cell to a signaling profile from a cell from an individual, e.g., a test subject (e.g., an undiagnosed individual), (vi) determining if the cell from the test subject e.g., an undiagnosed individual, is normal based on the comparing in (v), (vii) generating a report, (viii) modeling the dynamic response of nodes over time, (ix) characterizing the cells based on the activation levels over time (the “activation profile” of a node), (x) generating metrics such as slope or expressed using linear equations, (xi) segregating single cells into discrete cell populations, (xii) segregating a cell population based on a common characteristic including but not limited to: cell type, cell morphology and expression of a gene or protein, (xiii) simultaneously measuring the activation levels of several activatable elements in single cells, (xiv) measuring other markers (e.g., cell surface proteins, activatable elements) that can be used to determine a type of the cell, (xv) gating cells, (xvi) quantifying ranges of signaling of activatable elements within each cell sub-population, (xvii) describing signaling ranges within each sub-population for normal and diseased states by statistical methods such as histograms, boxplots, radar plots, a line graph with error bars, a bar and whisker plot, a circle plot, a heat map, and/or a bar graph, (xviii) using multivariate statistical methods, such as regression, random forests, or clustering, to summarize the ranges of signaling across all cell sub-populations for normal and diseased states, (xviv) normalizing a test sample based on a sample grouping or characteristic (e.g., race, age, ethnicity, or gender).

In some embodiments, methods include use of one or more computers in a computer system (1600). In some embodiments, the computer system is integrated into and is part of an analysis system, like a flow cytometer. In other embodiments, the computer system is connected to or ported to an analysis system. In some embodiments, the computer system is connected to an analysis system by a network connection. The computer may include a monitor 1607 or other graphical interface for displaying data, results, billing information, marketing information (e.g., demographics), customer information, or sample information. The computer may also include means for data or information input, such as a keyboard 1615 or mouse 1616. The computer may include a processing unit 1601 and fixed 1603 or removable 1611 media or a combination thereof. The computer may be accessed by a user in physical proximity to the computer, for example via a keyboard and/or mouse, or by a user 1622 that does not necessarily have access to the physical computer through a communication medium 1605 such as a modem, an internet connection, a telephone connection, or a wired or wireless communication signal carrier wave. In some cases, the computer may be connected to a server 1609 or other communication device for relaying information from a user to the computer or from the computer to a user. In some cases, the user may store data or information obtained from the computer through a communication medium 1605 on media, such as removable media 1612.

The computer executable logic can work in any computer that may be any of a variety of types of general-purpose computers such as a personal computer, network server, workstation, or other computer platform now or later developed. In some embodiments, a computer program product is described comprising a computer usable medium having the computer executable logic (computer software program, including program code) stored therein. The computer executable logic can be executed by a processor, causing the processor to perform functions described herein. In other embodiments, some functions are implemented primarily in hardware using, for example, a hardware state machine. Implementation of the hardware state machine so as to perform the functions described herein will be apparent to those skilled in the relevant arts. In some embodiments, a system is provided for executing computer executable logical, wherein the system comprises a computer.

The program can provide a method of determining the status of an individual by accessing data that reflects the activation level of one or more activatable elements in the reference population of cells.

### Conditions

The methods described herein can be applicable to any condition in an individual involving, indicated by, and/or arising from, in whole or in part, altered physiological status in cells. The term “physiological status” includes mechanical, physical, and biochemical functions in a cell. In some embodiments, the physiological status of a cell is determined by measuring characteristics of at least one cellular component of a cellular pathway in cells from different populations (e.g., different cell networks). Cellular pathways are well known in the art. In some embodiments the cellular pathway is a signaling pathway. Signaling pathways are also well known in the art (see, e.g., Hunter T., Cell 100(1): 113-27 (2000); Cell Signaling Technology, Inc., 2002 Catalogue, Pathway Diagrams pgs. 232-253; Weinberg, Chapter 6, The biology of Cancer, 2007; and Blume-Jensen and Hunter, Nature, vol 411, 17 May 2001, p 355-365). A condition involving or characterized by altered physiological status may be readily identified, for example, by determining the state of one or more activatable elements in cells from different populations, as taught herein.

In certain embodiments, the condition is a neoplastic, immunologic or hematopoietic condition. In some embodiments, the neoplastic, immunologic or hematopoietic condition is selected from the group consisting of solid tumors such as head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma, sepsis, allergic diseases and disorders that include but are not limited to allergic rhinitis, allergic conjunctivitis, allergic asthma, atopic eczema, atopic dermatitis, and food allergy, immunodeficiencies including but not limited to severe combined immunodeficiency (SCID), hypereosiniphic syndrome, chronic granulomatous disease, leukocyte adhesion deficiency I and II, hyper IgE syndrome, Chediak Higashi, neutrophilias, neutropenias, aplasias, agammaglobulinemia, hyper-IgM syndromes, DiGeorge/Velocardial-facial syndromes and Interferon gamma-TH1 pathway defects, autoimmune and immune dysregulation disorders that include but are not limited to rheumatoid arthritis, diabetes, systemic lupus erythematosus, Graves' disease, Graves ophthalmopathy, Crohn's disease, multiple sclerosis, psoriasis, systemic sclerosis, goiter and struma lymphomatosa (Hashimoto's thyroiditis, lymphadenoid goiter), alopecia aerata, autoimmune myocarditis, lichen sclerosis, autoimmune uveitis, Addison's disease, atrophic gastritis, myasthenia gravis, idiopathic thrombocytopenic purpura, hemolytic anemia, primary biliary cirrhosis, Wegener's granulomatosis, polyarteritis nodosa, and inflammatory bowel disease, allograft rejection and tissue destructive from allergic reactions to infectious microorganisms or to environmental antigens, and hematopoietic conditions that include but are not limited to Non-Hodgkin Lymphoma, Hodgkin or other lymphomas, acute or chronic leukemias, polycythemias, thrombocythemias, multiple myeloma or plasma cell disorders, e.g., amyloidosis and Waldenstrom's macroglobulinemia, myelodysplastic disorders, myeloproliferative disorders, myelofibroses, or atypical immune lymphoproliferations. In some embodiments, the neoplastic or hematopoietic condition is non-B lineage derived, such as Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, or non-B atypical immune lymphoproliferations, Chronic Lymphocytic Leukemia (CLL), B lymphocyte lineage leukemia, B lymphocyte lineage lymphoma, Multiple Myeloma, or plasma cell disorders, e.g., amyloidosis or Waldenstrom's macroglobulinemia.

In some embodiments, the neoplastic or hematopoietic condition is non-B lineage derived. Examples of non-B lineage derived neoplastic or hematopoietic condition include, but are not limited to, Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B atypical immune lymphoproliferations.

In some embodiments, the neoplastic or hematopoietic condition is a B-Cell or B cell lineage derived disorder. Examples of B-Cell or B cell lineage derived neoplastic or hematopoietic condition include but are not limited to Chronic Lymphocytic Leukemia (CLL), B lymphocyte lineage leukemia, B lymphocyte lineage lymphoma, Multiple Myeloma, and plasma cell disorders, including amyloidosis and Waldenstrom's macroglobulinemia.

Other conditions can include, but are not limited to, cancers such as gliomas, lung cancer, colon cancer and prostate cancer. Specific signaling pathway alterations have been described for many cancers, including loss of PTEN and resulting activation of Akt signaling in prostate cancer (Whang Y E. Proc Natl Acad Sci USA Apr. 28, 1998; 95(9):5246-50), increased IGF-1 expression in prostate cancer (Schaefer et al., Science Oct. 9, 1998, 282: 199a), EGFR overexpression and resulting ERK activation in glioma cancer (Thomas C Y. Int J Cancer Mar. 10, 2003; 104(1):19-27), expression of HER2 in breast cancers (Menard et al. Oncogene. Sep. 29 2003, 22(42):6570-8), and APC mutation and activated Wnt signaling in colon cancer (Bienz M. Curr Opin Genet Dev 1999 October, 9(5):595-603).

In certain embodiments, the condition is neurological condition, e.g., Alzheimer's disease, Bell's Palsy, aphasia, Creutzfeldt-Jakob Disease (CJD), cerebrovascular disease, encephalitis, epilepsy, Huntington's disease, trigeminal neuralgia, migraine, Parkinson's disease, amyotrophic lateral sclerosis, Guillain-Barre syndrome, muscular dystrophy, spastic paraplegia, Von Hippel-Lindau disease (VHL), autism, dyslexia, narcolepsy, restless legs syndrome, Meniere's disease, or dementia.

Diseases other than cancer involving altered physiological status are also encompassed by the methods described herein. For example, it has been shown that diabetes involves underlying signaling changes, namely resistance to insulin and failure to activate downstream signaling through IRS (Burks D J, White M F. Diabetes 2001 February; 50 Suppl 1:S140-5). Similarly, cardiovascular disease has been shown to involve hypertrophy of the cardiac cells involving multiple pathways such as the PKC family (Malhotra A. Mol Cell Biochem 2001 September; 225 (1-):97-107). Inflammatory diseases, such as rheumatoid arthritis, are known to involve the chemokine receptors and disrupted downstream signaling (D'Ambrosio D. J Immunol Methods 2003 February; 273 (1-2):3-13). The methods described herein are not limited to diseases presently known to involve altered cellular function, but include diseases subsequently shown to involve physiological alterations or anomalies.

### Kits

In some embodiments, kits are provided. Kits may comprise one or more of the state-specific binding elements described herein, such as phospho-specific antibodies. A kit may also include other reagents, such as modulators, fixatives, containers, plates, buffers, therapeutic agents, instructions, and the like.

In some embodiments, the kit comprises one or more of the phospho-specific antibodies specific for the proteins selected from the group consisting of PI3-Kinase (p85, p110a, p110b, p110d), Jak1, Jak2, SOCs, Rac, Rho, Cdc42, Ras-GAP, Vav, Tiam, Sos, Dbl, Nck, Gab, PRK, SHP1, and SHP2, SHIP1, SHIP2, sSHIP, PTEN, Shc, Grb2, PDK1, SGK, Akt1, Akt2, Akt3, TSC1, 2, Rheb, mTor, 4EBP-1, p70S6Kinase, S6, LKB-1, AMPK, PFK, Acetyl-CoAa Carboxylase, DokS, Rafs, Mos, Tpl2, MEK1/2, MLK3, TAK, DLK, MKK3/6, MEKK1, 4, MLK3, ASK1, MKK4/7, SAPK/JNK1, 2, 3, p38s, Erk1/2, Syk, Btk, BLNK, LAT, ZAP70, Lck, Cbl, SLP-76, PLCγ₁, PLCγ2, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H₂B, HATs, HDACs, PKR, Rb, Cyclin D, Cyclin E, Cyclin A, Cyclin B, P16, p14Arf, p27KIP, p21CIP, Cdk4, Cdk6, Cdk7, Cdk1, Cdk2, Cdk9, Cdc25, A/B/C, Abl, E2F, FADD, TRADD, TRAF2, Myd88, BAD, Bcl-2, Mcl-1, Bcl-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(RelA), IKKα, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, \tilde{B}catenin, CrkL, GSK3α, GSK3β, and FOXO. In some embodiments, the kit comprises one or more of the phospho-specific antibodies specific for the proteins selected from the group consisting of Erk, Erk1, Erk2, Syk, Zap70, Lck, Btk, BLNK, Cbl, PLCγ2, Akt, Rel A, p38, S6. In some embodiments, the kit comprises one or more of the phospho-specific antibodies specific for the proteins selected from the group consisting of Akt1, Akt2, Akt3, SAPK/JNK1, 2, 3, p38s, Erk1/2, Syk, ZAP70, Btk, BLNK, Lck, PLCγ, PLCγ2, STAT1, STAT 3, STAT 4, STAT 5, STAT 6, CREB, Lyn, p-S6, Cbl, NF-kB, GSK3β, CARMA/Bcl10 and Tcl-1.

The state-specific binding element can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

### Generation of Dynamic Activation State Data

In some embodiments, the activation levels of a discrete cell population or a discrete subpopulation of cells may be measured at multiple time intervals following treatment with a modulator to generate “dynamic activation state data” (also referred to herein as kinetic activation state data). In these embodiments, a sample or sub-sample (e.g., patient sample) is divided into aliquots which are then treated with one or more modulators. The different aliquots can then be subject to treatment with a fixing agent at the different time intervals. For instance, an aliquot that is to be measured at 5 minutes can be treated with one or more modulators and can then be subjected to a treatment with a fixing agent after 5 minutes. The time intervals can vary greatly and can range from minutes (e.g., 5, 10, 15, 20, 25, 30, 35, 40, 45, 50, 55 minutes) to hours (e.g., 1, 2, 3, 4, 5, 6, 7, 8, 9, 10, 11, 12, 13, 14, 15, 6, 17 18, 19, 20, 21, 22, 23 hours) to days (e.g., 24 hours, 48 hours, 72 hours) or any combination thereof. Cells may also be treated with different concentrations of a modulator.

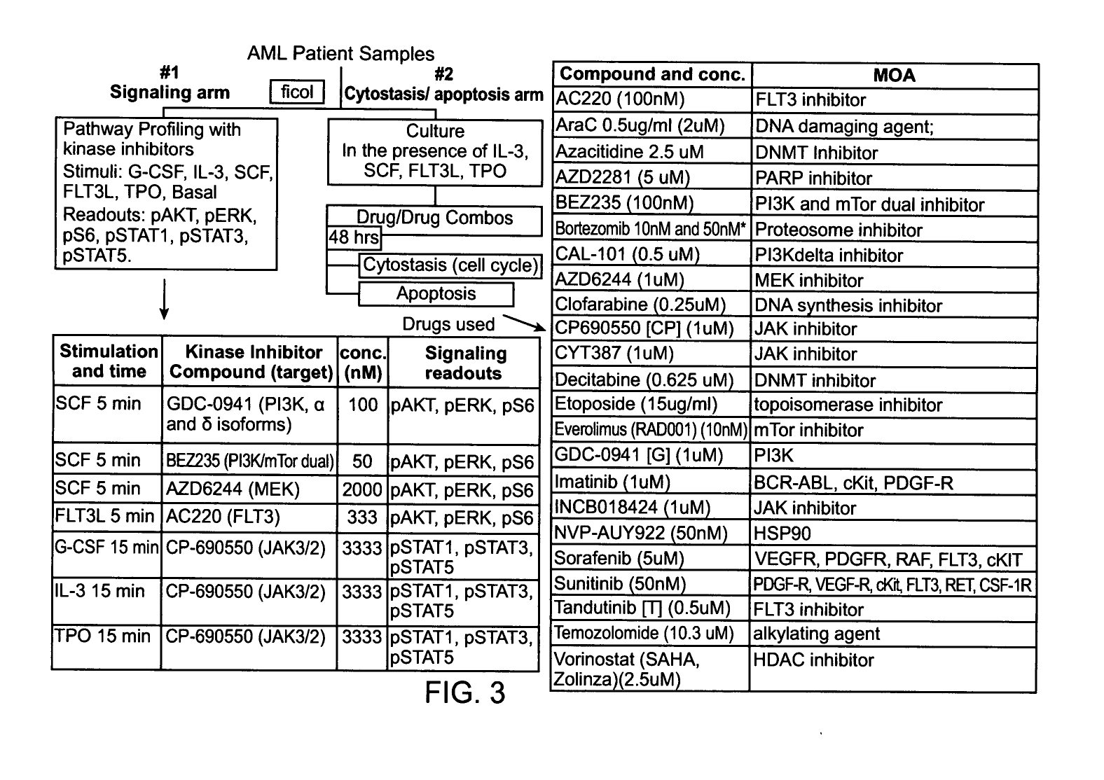

In some embodiments, the activation state data may be analyzed to identify discrete cell populations and then further analyzed to characterize the response of the different discrete cell populations to the modulator over time. The activation state data may be temporally modeled to characterize the dynamic response of the activatable elements to the stimulation with the modulator. Modeling the dynamic response to modulation can provide a better understanding of the patho-physiology of a disease or prognostic status or a response to treatment. Modeling the dynamic response of normal cells to a modulator is shown in FIG. 3 and discussed below with respect to Example 6. Additionally, the modulator-induced activation levels of a discrete population of cells over time associated with a disease status may be compared with other samples to identify activation levels that represent an aberrant response to a modulator at specific time points. Aberrant response to a modulator may be associated with health status, a prognostic status, a cytogenetic status or predicted therapeutic response. Having activation levels at different time points is beneficial because the maximal differential response between samples associated with different statuses may be observed as early as 5 minutes after treatment with a modulator and as late as 72 hours after treatment with a modulator.

The modulator-induced response of the different discrete cell populations may be modeled to further understand communication between the discrete cell populations that are associated with disease. For example, an increased phosphorylation of an activatable element in a first cell population at an earlier time point may have a causal effect on the phosphorylation of a second activatable element in a second cell population at a later time point. These causal associations may be modeled using Bayesian Networks or temporal models. These causal associations may be identified using unsupervised learning techniques such as principle components analysis and/or clustering. Causal associations between activation levels in different cell populations may represent communications between cellular networks over time. These communications may provide insight into the mechanism of drug response, cancer progression and carcinogenesis. Therefore, the identification and characterization of these communications allows for the development of diagnostics which can accurately predict drug response, therapeutic and early stage detection.

In some embodiments, the activation state data at a first time point is computationally analyzed (e.g., through binning or gating as described below) to determine discrete populations of cells. The discrete populations of cells are subsequently analyzed individually over the remaining time points to identify sub-populations of cells with different response to a modulator. Differential response over time within a same population of cells may be modeled using methods such as temporal modeling or hyper-spatial modeling as described in U.S. Patent Application 61/317,817 and below. These methods may allow the modeling of a single discrete cell population over time or multiple discrete cell populations over time.

In another embodiment, the activation state data is computational analyzed at all of the time points to determine discrete populations of cells. The discrete populations of cells can then be modeled in order to determine consistent membership in a discrete population of cells over time. In this way, the populations of cells are not characterized by the activation levels of modulators at a single time point, but rather can be determined based on the activation levels of modulators at multiple time points. Both gating and binning may be used to first segregate the activation state data for cell populations at all of the time points. Based on the segregated cell populations at the various time points, discrete cell populations may be identified. This technique works well using gating or semi-supervised identification of discrete cell populations, and the technique can be used with unsupervised identification of discrete cell populations such as the methods described in U.S. Publication No. 2009/0307248 and below.

### Computational Identification of Cell Populations

In some embodiments, the activation state data of a cell population is determined by contacting the cell population with one or more modulators, generating activation state data for the cell population and using computational techniques to identify one or more discrete cell populations based on the data. These techniques can be implemented using computers comprising memory and hardware. In one embodiment, algorithms for generating metrics based on raw activation state data are stored in the memory of a computer and executed by a processor of a computer. These algorithms can be used in conjunction with gating and binning algorithms, which can also be stored and executed by a computer, to identify the discrete cell populations.

The data can be analyzed using various metrics. For example, the median fluorescence intensity (MFI) can be computed for each activatable element from the intensity levels for the cells in the cell population gate. The MFI values can then be used to compute a variety of metrics by comparing them to the various baseline or background values, e.g., the unstimulated condition, autofluorescence, and isotype control. The following metrics are examples of metrics that can be used in the methods described herein: 1) a metric that measures the difference in the log of the median fluorescence value between an unstimulated fluorochrome-antibody stained sample and a sample that has not been treated with a stimulant or stained (log(MFI_{Unstimulated Stained})−log (MFI_{Gated Unstained})), 2) a metric that measures the difference in the log of the median fluorescence value between a stimulated fluorochrome-antibody stained sample and a sample that has not been treated with a stimulant or stained (log (MFI_{Stimulated Stained})−log (MFI_{Gated Unstained})), 3) a metric that measures the change between the stimulated fluorochrome-antibody stained sample and the unstimulated fluorochrome-antibody stained sample log (MFI_{Stimulated Stained})−log (MFI_{Unstumulated Stained}), also called “fold change in median fluorescence intensity”, 4) a metric that measures the percentage of cells in a Quadrant Gate of a contour plot which measures multiple populations in one or more dimension 5) a metric that measures MFI of phosphor positive population to obtain percentage positivity above the background and 6) use of multimodality and spread metrics for large sample population and for subpopulation analysis.

In a specific embodiment, the equivalent number of reference fluorophores value (ERF) is generated. The ERF is a transformed value of the median fluorescent intensity values. The ERF value is computed using a calibration line determined by fitting observations of a standardized set of &peak rainbow beads for all fluorescent channels to standardized values assigned by the manufacturer. The ERF values for different samples can be combined in any way to generate different activation state metric. Different metrics can include: 1) a fold value based on ERF values for samples that have been treated with a modulator (ERFₘ) and samples that have not been treated with a modulator (ERFᵤ), log₂ (ERFₘ/ERFᵤ); 2) a total phospho value based on ERF values for samples that have been treated with a modulator (ERFₘ) and samples from autofluorecsent wells (ERFₐ), log₂ (ERFₘ/ERFₐ); 3) a basal value based on ERF values for samples that have not been treated with a modulator (ERFᵤ) and samples from autofluorescent wells (ERFₐ), log₂ (ERFᵤ/ERFₐ); 4) A Mann-Whitney statistic Uᵤ comparing the ERF_{m and} ERFᵤ values that has been scaled down to a unit interval (0,1) allowing inter-sample comparisons; 5) A Mann-Whitney statistic Uᵤ comparing the ERFₘ and ERFᵤ values that has been scaled down to a unit interval (0,1) allowing inter-sample comparisons; 5) a Mann-Whitney statistic Uₐ comparing the ERFₐ and ERFₘ values that has been scaled down to a unit interval (0,1); and 6) A Mann-Whitney statistic U75. U75 is a linear rank statistic designed to identify a shift in the upper quartile of the distribution of ERFₘ and ERFᵤ values. ERF values at or below the 75^{th} percentile of the ERFₘ and ERFᵤ values are assigned a score of 0. The remaining ERFₘ and ERFᵤ values are assigned values between 0 and 1 as in the Uᵤ statistic. For activatable elements that are surface markers on cells, the following metrics may be further generated: 1) a relative protein expression metric log 2(ERFₛₜₐᵢₙ)−log 2(ERF_{control}) based on the ERF value for a stained sample (ERFₛₜₐᵢₙ) and the ERF value for a control sample (ERF_{control}); and 2) A Mann-Whitney statistic Ui comparing the ERFₘ and ERFᵢ values that has been scaled down to a unit interval (0,1), where the ERFᵢ values are derived from an isotype control.

The activation state data for the different markers can be “gated” in order to identify discrete subpopulations of cells within the data. In gating, activation state data can be used to identify discrete sub-populations of cells with distinct activation levels of an activatable element. These discrete sub-populations of cells can correspond to cell types, cell sub-types, cells in a disease or other physiological state and/or a population of cells having any characteristic in common.

In some embodiments, the activation state data is displayed as a two-dimensional scatter-plot and the discrete subpopulations are “gated” or demarcated within the scatter-plot. According to the embodiment, the discrete subpopulations may be gated automatically, manually or using some combination of automatic and manual gating methods. In some embodiments, a user can create or manually adjust the demarcations or “gates” to generate new discrete sub-populations of cells. Suitable methods of gating discrete sub-populations of cells are described in U.S. patent application Ser. No. 12/501,295, the entirety of which is incorporated by reference herein, for all purposes.

In some embodiments, the homogenous cell populations are gated according to markers that are known to segregate different cell types or cell sub-types. In a specific embodiment, a user can identify discrete cell populations based on surface markers. For example, the user could look at: “stem cell populations” by CD34+ CD38- or CD34+ CD33-expressing cells; memory CD4 T lymphocytes; e.g., CD4⁺ CD45RA⁺ CD29^{low} cells; or multiple leukemic sub-clones based on CD33, CD45, HLA-DR, CD11b and analyzing signaling in each discrete population/subpopulation. In another embodiment, a user may identify discrete cell populations/subpopulations based on intracellular markers, such as transcription factors or other intracellular proteins; based on a functional assay (e.g., dye efflux assay to determine drug transporter+cells or fluorescent glucose uptake) or based on other fluorescent markers. In some embodiments, gates are used to identify the presence of specific discrete populations and/or subpopulations in existing independent data. The existing independent data can be data stored in a computer from a previous patient, or data from independent studies using different patients.

In some embodiments, the homogenous cell populations/subpopulations are automatically gated according to activation state data that segregates the cells into discrete populations. For example, an activatable element that is “on” or “off” in cells may be used to segregate the cell population into two discrete subpopulations. In embodiments where the discrete cell subpopulations are automatically identified, different algorithms may be used to identify discrete homogenous cell subpopulations based on the activation state data. In a specific embodiment, a multi-resolution binning algorithm is used to iteratively identify discrete subpopulations of cells by partitioning the activation state data. This algorithm is outlined in detail in U.S. Publication No. 2009/0307248, which is incorporated herein in its entirety, for all purposes. In one embodiment, the multi-resolution binning algorithm is used to identify rare or uniquely discrete cell populations by iteratively identifying vectors or “hyperplanes” that partition activation state data into finer resolution bins. Using iterative algorithms such as multi-resolution binning algorithms, fine resolution bins containing rare populations of cells may be identified. For example, activation state data for one or more markers may be iteratively binned to identify a small number of cells with an unusually high expression of a marker. Normally, these cells would be discarded as “outlier” data or during normalization of the data. However, multi-resolution binning allows the identification of activation state data corresponding to rare populations of cells.

In different embodiments, gating can be used in different ways to identify discrete cell populations. In one embodiment, “Outside-in” comparison of activation state data for individual samples or subset (e.g., patients in a trial) is used to identify discrete cell populations. In this embodiment, cell populations are homogenous or lineage gated in such a way as to create discrete sets of cells considered to be homogenous based on a characteristic (e.g., cell type, expression, subtype, etc.). An example of sample-level comparison in an AML patient would be the identification of signaling profiles in lymphocytes (e.g., CD4 T cells, CD8 T cells and/or B cells), monocytes+granulocytes and leukemic blast and correlating the activation state data of these populations with non-random distribution of clinical responses. This is considered an outside-in approach because the discrete cell population of interest is pre-defined prior to the mapping and comparison of its profile to, e.g., a clinical outcome or the profile of the populations in normal individuals.

In other embodiments, “Inside-out” comparison of activation state data at the level of individual cells in a heterogeneous population is used to identify discrete cell populations. An example of this method would be the signal transduction state mapping of mixed hematopoietic cells under certain conditions and subsequent comparison of computationally identified cell clusters with lineage specific markers. This method could be considered an inside-out approach to single cell studies as it does not presume the existence of specific discrete cell populations prior to classification. Suitable methods for inside-out identification of discrete cell populations include the multi-resolution binning algorithm described above. This approach can create discrete cell populations which, at least initially, can use multiple transient markers to enumerate and may never be accessible with a single cell surface epitope. As a result, the biological significance of such discrete cell populations can be difficult to determine. The main advantage of this unconventional approach is the unbiased tracking of discrete cell populations without drawing potentially arbitrary distinctions between lineages or cell types and the potential of using the activation state data of the different populations to determine the status of an individual.

Classifying and Characterizing Cell Network Based on Activation State Data Associated with Discrete Populations of Cells

When the activation state data associated with a plurality of discrete cell populations has been identified, it can be useful to determine whether activation state data is non-randomly distributed within the categories such as disease status, therapeutic response, clinical responses, presence of gene mutations, and protein expression levels. Activation state data that are strongly associated with one or more discrete cell populations with a specific characteristic (e.g., gene mutation, disease status) can be used both to classify a cell according to the characteristic and to further characterize and understand the cell network communications underlying the pathophysiology of the characteristic. Activation state data that uniquely identifies a discrete cell population associated with a cell network can serve to re-enforce or complement other activation state data that uniquely identifies another discrete cell population associated with the cell network.

If activation state data is available for many discrete cell populations, activation state data that uniquely identifies a discrete cell population may be identified using simple statistical tests, such as the Student's t-test and the X² test. Similarly, if the activation state data of two discrete cell populations within the experiment is thought to be related, the r² correlation coefficient from a linear regression can be used to represent the degree of this relationship. Other methods include Pearson and Spearman rank correlation. In some embodiments, correlation and statistical test algorithms will be stored in the memory of a computer and executed by a processor associated with the computer.

In some embodiments, the invention provides methods for determining whether the activation state data of different discrete cell populations is associated with a cellular network and/or a characteristic that can potentially complement each other to improve the accuracy of classification. In these embodiments, the activation state data of the discrete cell populations may be used generate a classifier for one or more characteristics associated with the discrete cell populations including but not limited to: therapeutic response, disease status and disease prognosis. A classifier can be any type of statistical model that can be used to characterize a similarity between a sample and a class of samples. Classifiers can comprise binary and multi-class classifiers as in the traditional use of the term classifier. Classifiers can also comprise statistical models of activation levels and variance in only one class of samples (e.g., normal individuals). These single-class classifiers can be applied to data, e.g., from undiagnosed samples, to produce a similarity value, which can be used to determine whether the undiagnosed sample belongs to the class of samples (e.g. by using a threshold similarity value). Any suitable method known in the art can be used to generate the classifier. For example, simple statistical tests can be used to generate a classifier. Examples, of classification algorithms that can be used to generate a classifier include, but are not limited to, Linear classifiers, Fisher's linear discriminant, ANOVA, Logistic regression, Naive Bayes classifier, Perceptron, Support vector machines, Quadratic classifiers, Kernel estimation, k-nearest neighbor, Boosting. Decision trees, Random forests, Neural networks, Bayesian networks, Hidden Markov models, and Learning vector quantization. Thus, in some embodiments, different types of classification algorithms may be used to generate the classifier including but not limited to: neural networks, support vector machines (SVMs), bagging, boosting and logistic regression. In some embodiments, the activation state data for different discrete populations associated with a same network and/or characteristic may be pooled before generating a classifier that specifies which combinations of activation state data associated with discrete cell populations can be used to uniquely identify and classify cells according to the activatable element.

In a specific embodiment, if the size of the activation state data associated with the discrete populations of cells is small, a straightforward corner classifier approach for picking combinations of activation state data that uniquely identifies the different discrete cell populations can be adopted. Combinations of discrete cell populations' activation state data can also be tested for their stability via a bootstrapping approach described below. In this embodiment, a corners classification algorithm can be applied to the data. The corners classifier is a rules-based algorithm for dividing subjects into two classes (e.g. dichotomized response to a treatment) using one or more numeric variables (e.g. population/node combination). This method works by setting a threshold on each variable, and then combining the resulting intervals (e.g., X<10, or Y>50) with the conjunction (and) operator (reference). This creates a rectangular region that is expected to hold most members of the class previously identified as the target (e.g., responders or non-responders of treatment). Threshold values are chosen by minimizing an error criterion based on the logit-transformed misclassification rate within each class. The method assumes only that the two classes (e.g. response or lack of response to treatment) tend to have different locations along the variables used, and is invariant under monotone transformations of those variables.

In some embodiments, computational methods of cross-validation are used during classifier generation to measure the accuracy of the classifier and prevent over-fitting of the classifier to the data. In a specific embodiment, bagging techniques, aka bootstrapped aggregation, are used to internally cross-validate the results of the above statistical model. In this embodiment, re-samples are iteratively drawn from the original data and used to validate the classifier. Each classifier, e.g. combination of population/node, is fit to the resample, and used to predict the class membership of those patients who were excluded from the resample. The accuracy of false positive and false negative classifications is determined for each classifier.

After iteratively re-sampling the original data, each patient acquires a list of predicted class memberships based on classifiers that were fit using other patients. Each patient's list is reduced to the fraction of target class predictions; members of the target class should have fractions near 1, unlike members of the other class. The set of such fractions, along with the patient's true class membership, is used to create a Receiver Operator Curve and to calculate the area under the ROC curve (herein referred to as the “AUC”).

In some embodiments, methods are provided for determining a status of an individual such a disease status, therapeutic response, and/or clinical responses wherein the positive predictive value (PPV) is higher than 60, 70, 80, 90, 95, or 99.9%. In some embodiments, methods are provided for determining a status of an individual such as disease status, therapeutic response, and/or clinical responses, wherein the PPV is equal or higher than 95%. In some embodiments, methods are provided for determining a status of an individual such a disease status, therapeutic response, and/or clinical responses, wherein the negative predictive value (NPV) is higher than 60, 70, 80, 90, 95, or 99.9%. In some embodiments, methods are provided for determining a status of an individual such as disease status, therapeutic response, and/or clinical responses, wherein the NPV is higher than 85%.

In some embodiments, methods are provided for predicting risk of relapse at 2 years, wherein the PPV is higher than 60, 70, 80, 90, 95, or 99.9%. In some embodiments, methods are provided for predicting risk of relapse at 2 years, wherein the PPV is equal or higher than 95%. In some embodiments, methods are provided for predicting risk of relapse at 2 years, wherein the NPV is higher than 60, 70, 80, 90, 95, or 99.9%. In some embodiments, methods are provided for predicting risk of relapse at 2 years, wherein the NPV is higher than 80%. In some embodiments, methods are provided for predicting risk of relapse at 5 years, wherein the PPV is higher than 60, 70, 80, 90, 95, or 99.9%. In some embodiments, methods are provided for predicting risk of relapse at 5 years, wherein the PPV is equal or higher than 95%. In some embodiments, methods are provided for predicting risk of relapse at 5 years, wherein the NPV is higher than 60, 70, 80, 90, 95, or 99.9%. In some embodiments, methods are provided for predicting risk of relapse at 5 years, wherein the NPV is higher than 80%. In some embodiments, methods are provided for predicting risk of relapse at 10 years, wherein the PPV is higher than 60, 70, 80, 90, 95, or 99.9%. In some embodiments, methods are provided for predicting risk of relapse at 10 years, wherein the PPV is equal or higher than 95%. In some embodiments, methods are provided for predicting risk of relapse at 10 years, wherein the NPV is higher than 60, 70, 80, 90, 95, or 99.9%. In some embodiments, methods are provided for predicting risk of relapse at 10 years, wherein the NPV is higher than 80%.

In some embodiments, the p value in the analysis of the methods described herein is below 0.05, 04, 0.03, 0.02, 0.01, 0.009, 0.005, or 0.001. In some embodiments, the p value is below 0.001. Thus in some embodiments, methods are provided for determining a status of an individual such a disease status, therapeutic response, and/or clinical responses, wherein the p value is below 0.05, 04, 0.03, 0.02, 0.01, 0.009, 0.005, or 0.001. In some embodiments, the p value is below 0.001. In some embodiments, methods are provided for determining a status of an individual such a disease status, therapeutic response, and/or clinical responses, wherein the AUC value is higher than 0.5, 0.6, 07, 0.8 or 0.9. In some embodiments, methods are provided for determining a status of an individual such a disease status, therapeutic response, and/or clinical responses, wherein the AUC value is higher than 0.7. In some embodiments, methods are provided for determining a status of an individual such a disease status, therapeutic response, and/or clinical responses, wherein the AUC value is higher than 0.8. In some embodiments, methods are provided for determining a status of an individual such a disease status, therapeutic response, and/or clinical responses, wherein the AUC value is higher than 0.9.

In another embodiment, activation state data generated for a cellular network over a series of time points can be used to identify activation state data that represents unique communications within the cellular network over time. The activation state data that represents unique communications within the cellular network can be used to classify other activation state data associated with cell populations to determine whether they are associated with a same characteristic as the cellular network or determine if there are in a specific stage or phase in time that is unique to a cellular network. For example, different discrete populations of cells in a cellular network can be treated with a same modulator and sub-sampled over a series of time points to determine communications between the discrete populations of cells that are unique to the stimulation with the modulator. Similarly, samples of different discrete cell populations can be derived from patients over the course of treatment and used to identify communications between the discrete populations of cells that are unique to the course of treatment.

In one embodiment, the activation state data for a discrete cell population at different time points can be modeled to represent dynamic interactions between the discrete cell populations in a cell networks over time. The activation state data can be modeled using temporal models, Bayesian networks or some combination therefore. Suitable methods of generating Bayesian networks are described in 11/338,957, the entirety of which is incorporated herein, for all purposes. Suitable methods of generating temporal models of activation state data are described in U.S. Patent Application 61/317,817, the entirety of which is incorporated herein by reference. Different metrics may be generated to describe the dynamic interactions including: derivatives, integrals, rate-of-change metrics, splines, state representations of activation state data and Boolean representations of activation state data.

In embodiments where metrics and other values describing dynamic interactions are generated, these values and metrics are used to generate a classifier. As outlined above, any suitable classification algorithm can be used to determine metrics and values that uniquely identify cellular network data that shares a same characteristic. In some embodiments, the descriptive values and metrics will be generated based on two distinct data sets: 1) activation state data that is associated with a characteristic and 2) activation state data that is not association with a characteristic. For example: activation state data generated from discrete cell populations after stimulation with a modulator and activation state data generated from un-stimulated discrete cell populations. In these embodiments, the descriptive values and metrics will be used to generate a two-class classifier. In other embodiments, descriptive values and metrics will be generated from a large number of activation state data sets associated with different characteristics and a multi-class classifier will be generated. The resulting classifier will be used to determine whether a cellular network is part of the data set.

In some embodiments, the above classifiers are used to characterize activation state data derived from an individual such as a patient. In these embodiments, activation state data associated with a cellular network of one or more discrete cell populations is derived from a patient. In some embodiments, the activation state data associated with the different discrete cell populations from a patient may be identified by obtaining patient samples with different characteristics (e.g. blood cells and tumor samples). In some embodiments, the activation state data associated with the different discrete cell populations may be identified computationally based on activation state data for activatable elements that are known to differentiate discrete cell populations. A classifier that specifies activation state data from different discrete cell populations used to determine whether the cells have a common characteristic is applied to the activation state data associated with the individual in order to generate a classification value that specifies the probability that the individual (or the cells derived from the individual) is associated with the characteristic. In most embodiments, the classifier is stored in computer memory or computer-readable storage media as a set of values or executable code and applying the classifier comprises executing code that applies the classifier to the activation state data associated with the individual. The classification value may be output to a user, transmit to an entity requesting the classification value and/or stored in memory associated with a computer. The classification value may represent information related to or representing the physiological status of the individual such as a diagnosis, a prognosis or a predicted response to treatment.

In some embodiments, the activation state data of a plurality of cell populations is determined in normal individuals or individual not suffering or not suspected of suffering from a condition. This activation state data can be used to create statistical model of the ranges of activation levels observed in cell populations derived from samples obtained from normal patients (e.g. regression model, variance model). This ranges and/or models may be used to determine whether samples from undiagnosed individuals exhibit the range of activation state data observed in normal samples (e.g., range of normal activation levels). This can be used to create a classifier for normal individuals. In some embodiments, the models may be used to generate a similarity value that indicates the similarity of the activation state data associated with the undiagnosed individual to the range of normal activation levels (e.g. correlation coefficient, fitting metric) and/or a probability value that indicates the probability that the activation state data would be similar to the range of normal activation levels by chance (i.e. probability value and/or associated confidence value). In other embodiments, activation state data from normal patients may be combined with activation state data from patients that are known to have a disease to create a binary or multi-class classifier. In some embodiments, the activation state data from an undiagnosed individual will be displayed graphically with the range of activation states observed in normal cells. This allows for a person, for example a physician, to visually assess the similarity of the activation state data associated with the undiagnosed patient to that range of activation states observed in samples from normal individuals.

In one embodiment, a clinical decision can be made based on a similarity value. In one embodiment, a clinical decision can be a diagnosis, prognosis, course of treatment, or monitoring of a subject.

In some embodiments, methods are provided for evaluating cells that may be cancerous. The cells are subjected to the methods described herein and compared to a population of normal cells. The comparison can be done with any of the algorithms described herein. In some embodiments, the activation state data is represented in graphical form. Typically, when shown in a graph, normal cells have a uniform population and appear tightly grouped with narrow boundaries. When cancerous or pre-cancerous cells are subject to the same methods as normal cells (e.g., treatment with one or more modulators) and are represented on the same graph, deviations from the norm shown by the graph indicate a more heterogeneous population. This change is an indication that the cells may be cancerous in a manner that is a function of the degree of change. Morphology change may indicate a cancerous population on a continuation from mild to metastatic. If there is no shape change from normal, then there may not be a change in the cell phenotype.

The presence of a heterogeneous population of cells may indicate that therapy is needed. The outcome of the therapy can be monitored by reference to the graph. A change from a more heterogeneous population to a population that is more tightly grouped on the chart may indicate that the cell population is returning to a normal state. The lack of change may indicate that the therapy is not working and the cell population is refractory or resistant to therapy. It may also indicate that a different discrete cell population has changed over to the cancerous phenotype. Lack of change back to normal is indicative of a negative correlation to therapy. These changes may be genetic or epigenetic.

One embodiment of the present invention is to conduct the methods described herein by analyzing a population of normal cells to create a pattern or a database that can be compared in a graphical way to a cell population that is potentially cancerous. The analysis can be by many methods, but one preferred method is the use of flow cytometry.

In all these embodiments, the activation state data may be generated at a central laboratory and the classifier may be applied to the data at the central laboratory. Alternately, the activation state data may be generate by a third party and transmitted, for example, via a secure network to a central laboratory for classification. Methods of transmitting data for classification and analysis are outlined in U.S. patent application Ser. No. 12/688,851, the entirety of which is incorporated herein by reference, for all purposes.

### Methods

The methods described herein are suitable for any condition for which a correlation between the cell signaling profile of a cell and the determination of a disease predisposition, diagnosis, prognosis, and/or course of treatment in samples from individuals may be ascertained. In some embodiments, the methods described herein are directed to methods for analysis, drug screening, diagnosis, prognosis, and for methods of disease treatment and prediction. In some embodiments, the methods described herein comprise methods of analyzing experimental data. In some embodiments, the cell signaling profile of a cell population comprising a genetic alteration is used, e.g., in diagnosis or prognosis of a condition, patient selection for therapy, e.g., using some of the agents identified herein, to monitor treatment, modify therapeutic regimens, and/or to further optimize the selection of therapeutic agents which may be administered as one or a combination of agents. In some embodiments, the cell population is not associated and/or is not causative of the condition. In some embodiments, the cell population is associated with the condition but it has not yet developed the condition. The cell signaling profile of a cell population can be determined by determining the activation level of at least one activatable element in response to at least one modulator in one or more cells belonging to the cell population. The cell signaling profile of a cell population can be determined by adjusting the profile based on the presence of unhealthy cells in a sample.

In one embodiment, the methods described herein can be used to prevent disease, e.g., cancer by identifying a predisposition to the disease for which a medical intervention is available. In another embodiment, an individual afflicted with a condition can be treated. In another embodiment, methods are provided for assigning an individual to a risk group. In another embodiment, methods of predicting the increased risk of relapse of a condition are provided. In another embodiment, methods of predicting the risk of developing secondary complications are provided. In another embodiment, methods of choosing a therapy for an individual are provided. In another embodiment, methods of predicting the duration of response to a therapy are provided. In another embodiment, methods are provided for predicting a response to a therapy. In another embodiment, methods are provided for determining the efficacy of a therapy in an individual. In another embodiment, methods are provided for determining the prognosis for an individual.

The cell signaling profile of a cell population can serve as a prognostic indicator of the course of a condition, e.g. whether a person will develop a certain tumor or other pathologic conditions, whether the course of a neoplastic or a hematopoietic condition in an individual will be aggressive or indolent. The prognostic indicator can aid a healthcare provider, e.g., a clinician, in managing healthcare for the person and in evaluating one or more modalities of treatment that can be used. In another embodiment, the methods provided herein provide information to a healthcare provider, e.g., a physician, to aid in the clinical management of a person so that the information may be translated into action, including treatment, prognosis or prediction.

In some embodiments, the methods described herein are used to screen candidate compounds useful in the treatment of a condition or to identify new druggable targets.

In another embodiment, the cell signaling profile of a cell population can be used to confirm or refute a diagnosis of a pre-pathological or pathological condition.

In instances where an individual has a known pre-pathologic or pathologic condition, the cell signaling profile of the cell population can be used to predict the response of the individual to available treatment options. In one embodiment, an individual treated with the intent to reduce in number or ablate cells that are causative or associated with a pre-pathological or pathological condition can be monitored to assess the decrease in such cells and the state of a cellular network over time. A reduction in causative or associated cells may or may not be associated with the disappearance or lessening of disease symptoms. If the anticipated decrease in cell number and/or improvement in the state of a cellular network do not occur, further treatment with the same or a different treatment regiment may be warranted.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regime can be considered.

In a further embodiment, cells of an individual can be analyzed to see if treatment with a differentiating agent has pushed a cell type along a specific tissue lineage and to terminally differentiate with subsequent loss of proliferative or renewal capacity. Such treatment may be used preventively to keep the number of dedifferentiated cells associated with disease at a low level, thereby preventing the development of overt disease. Alternatively, such treatment may be used in regenerative medicine to coax or direct pluripotent or multipotent stem cells down a desired tissue or organ specific lineage and thereby accelerate or improve the healing process.

Individuals may also be monitored for the appearance or increase in cell number of another cell population(s) that are associated with a good prognosis. If a beneficial population of cells is observed, measures can be taken to further increase their numbers, such as the administration of growth factors. Alternatively, individuals may be monitored for the appearance or increase in cell number of another cells population(s) associated with a poor prognosis. In such a situation, renewed therapy can be considered including continuing, modifying the present therapy or initiating another type of therapy.

### Reports and Computers

In some embodiments, a report can be generated that can be used to communicate a signaling pathway activity in single cells, identify signaling pathway disruptions in diseased cells, including rare cell populations, identify response and resistant biological profiles that guide the selection of therapeutic regimens, monitor the effects of therapeutic treatments on signaling in diseased cells, and/or monitor the effects of treatment over time. A report can enable biology-driven patient management and drug development, improve patient outcome, reduce inefficient uses of resources, and improve speed of drug development cycles.

A report can compare a signaling profile from one or more normal cells to a signaling profile from a test subject, e.g., a patient, e.g., an undiagnosed individual. A report can compare an activation level of one or more activable elements from one or more normal cells to an activation level of the one or more activable elements from a cell from a test subject, e.g., a patient, e.g., an undiagnosed individual.

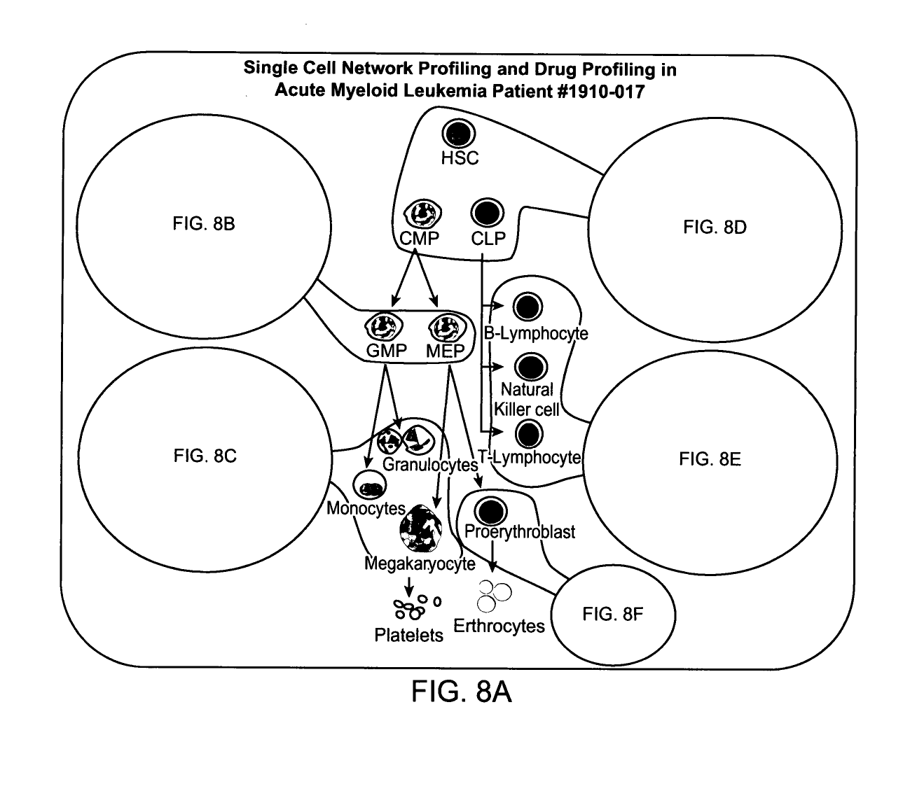

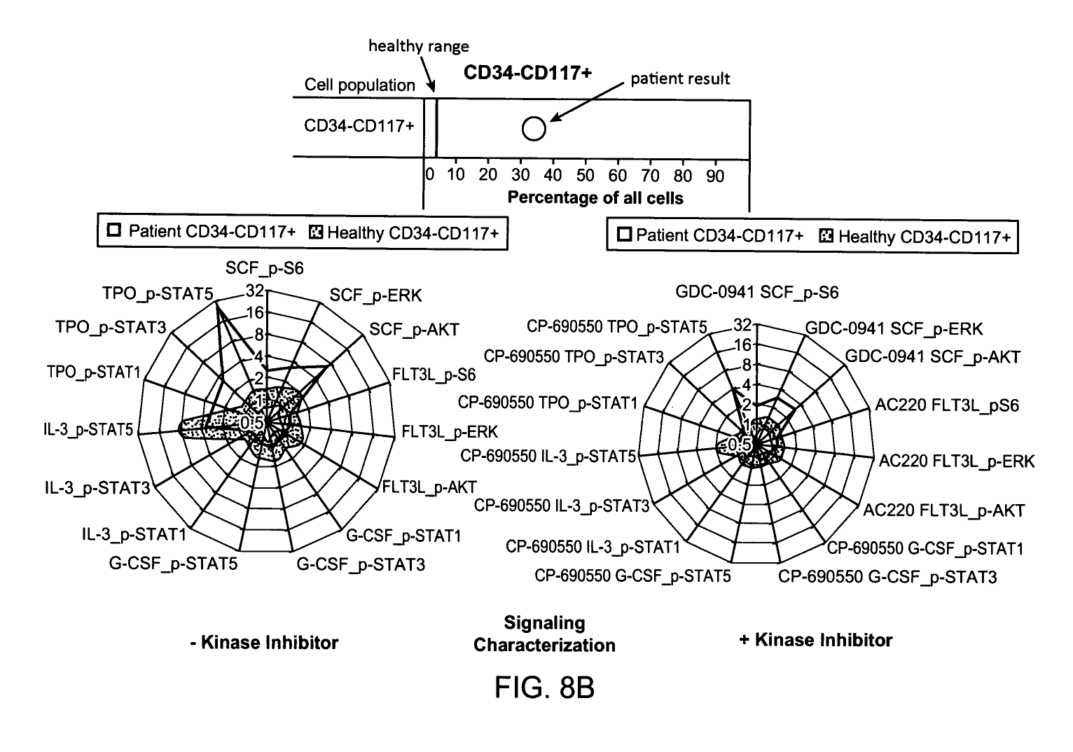

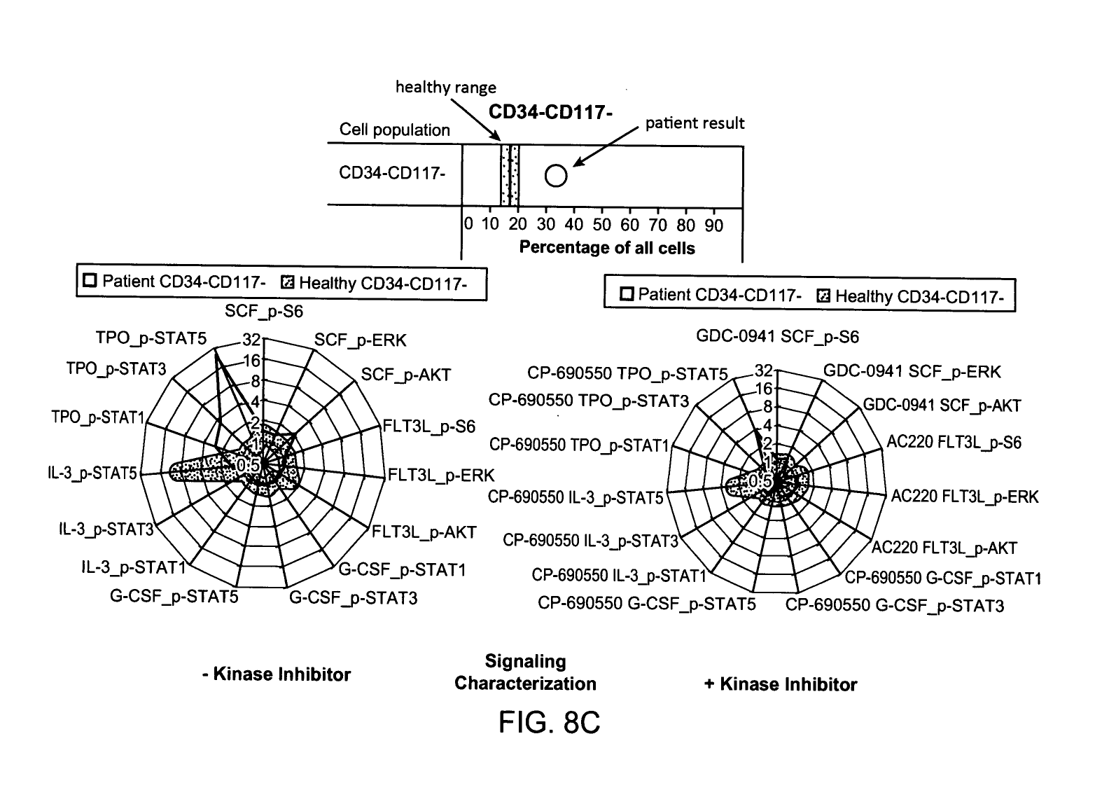

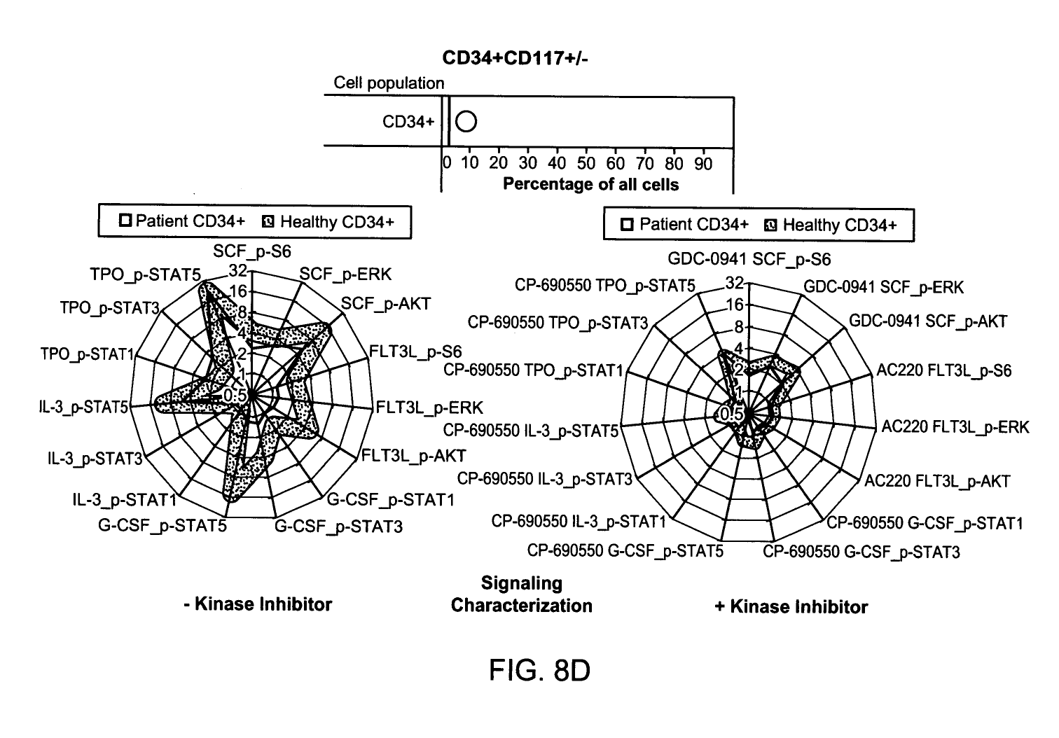

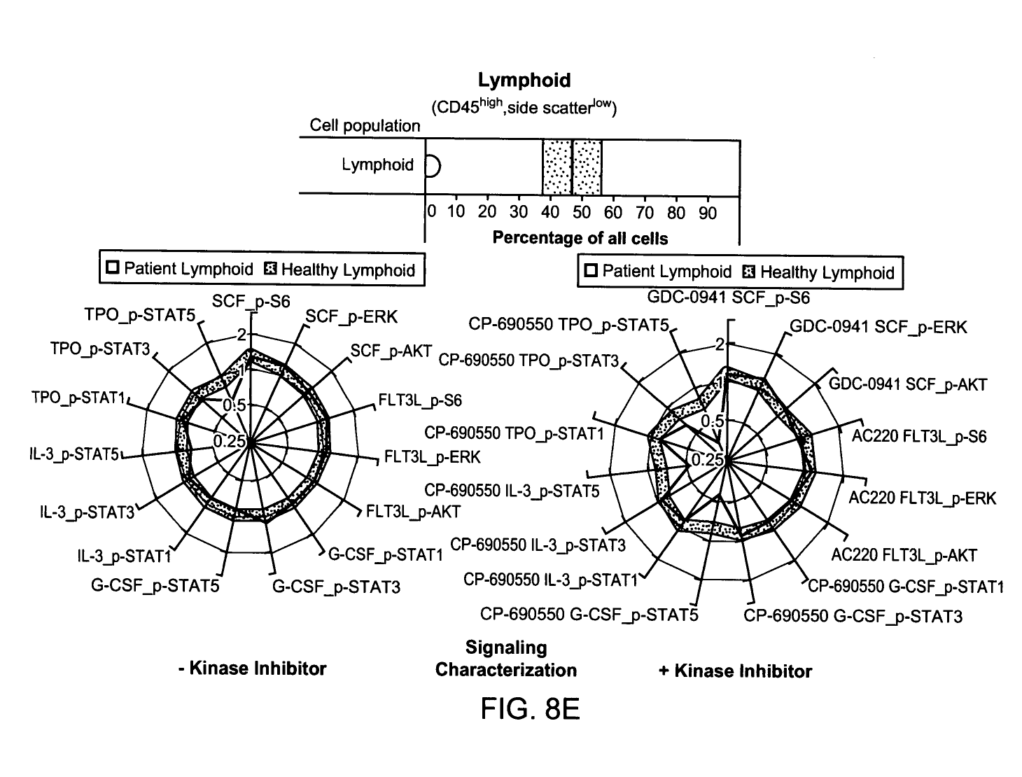

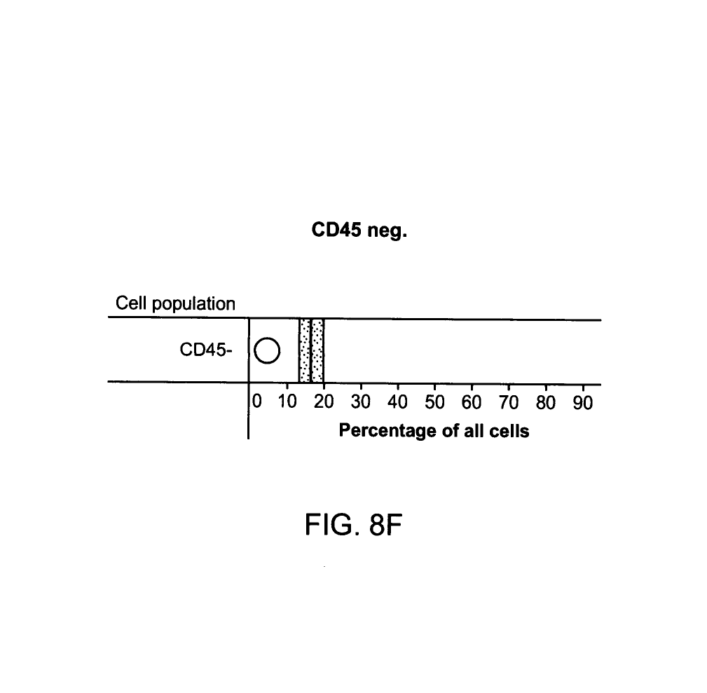

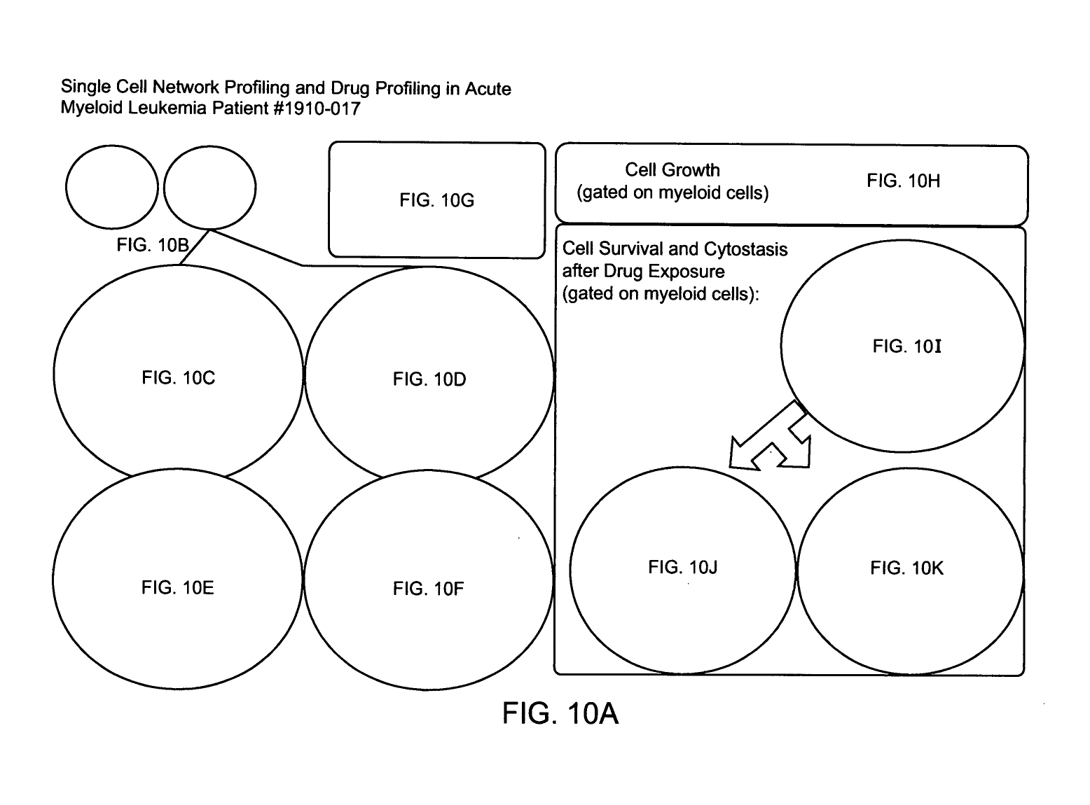

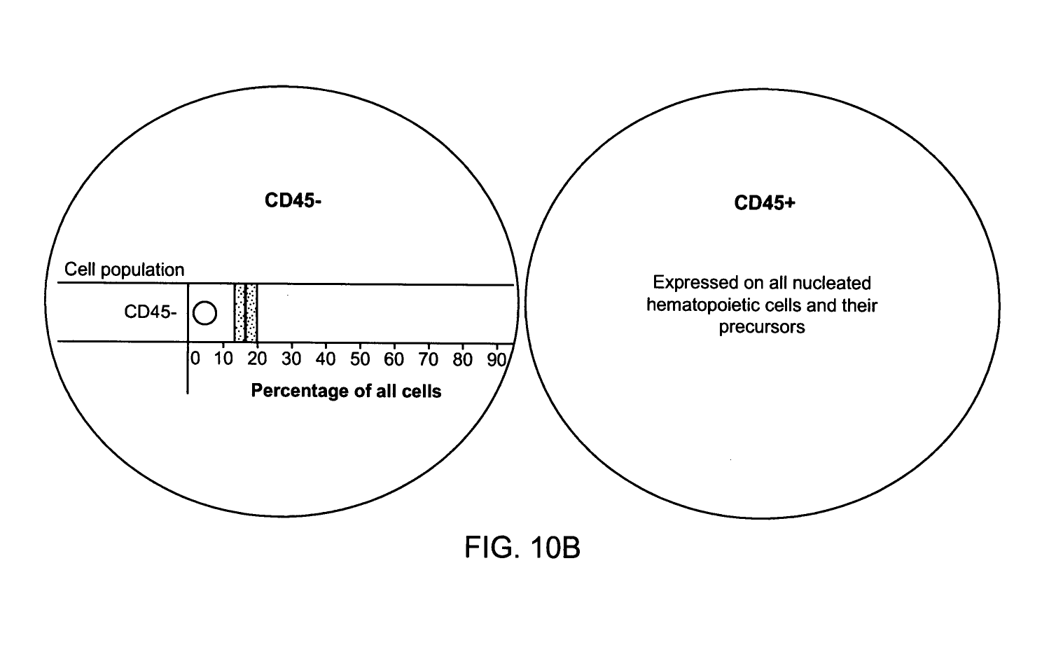

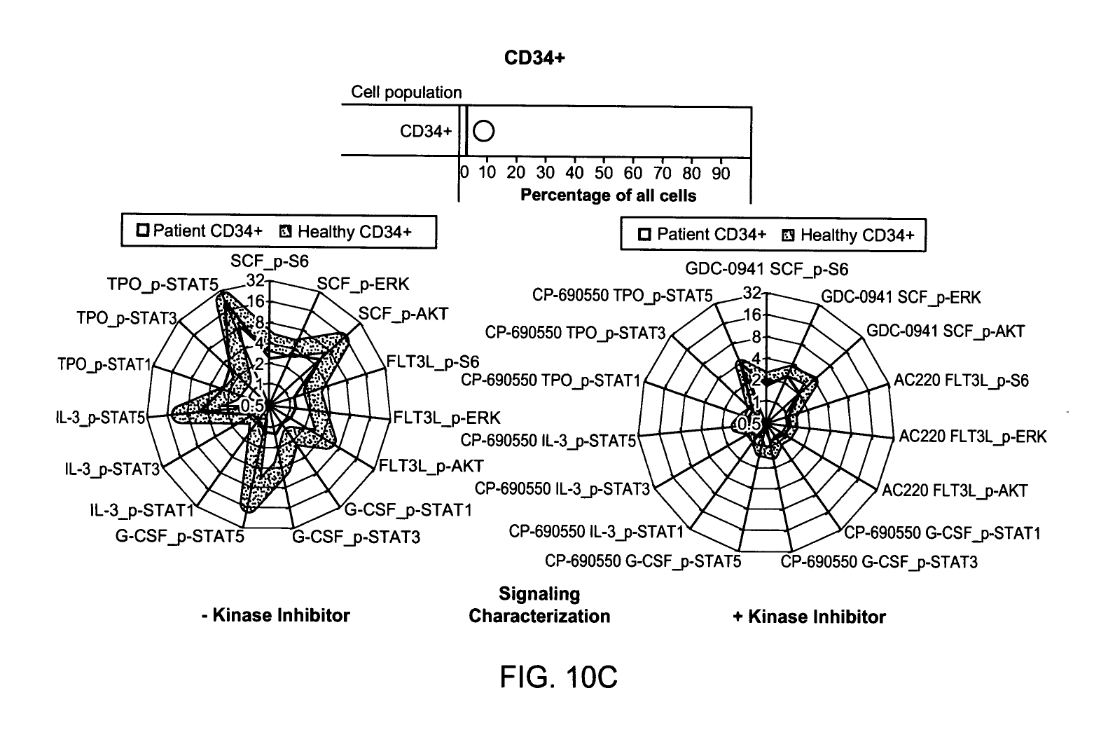

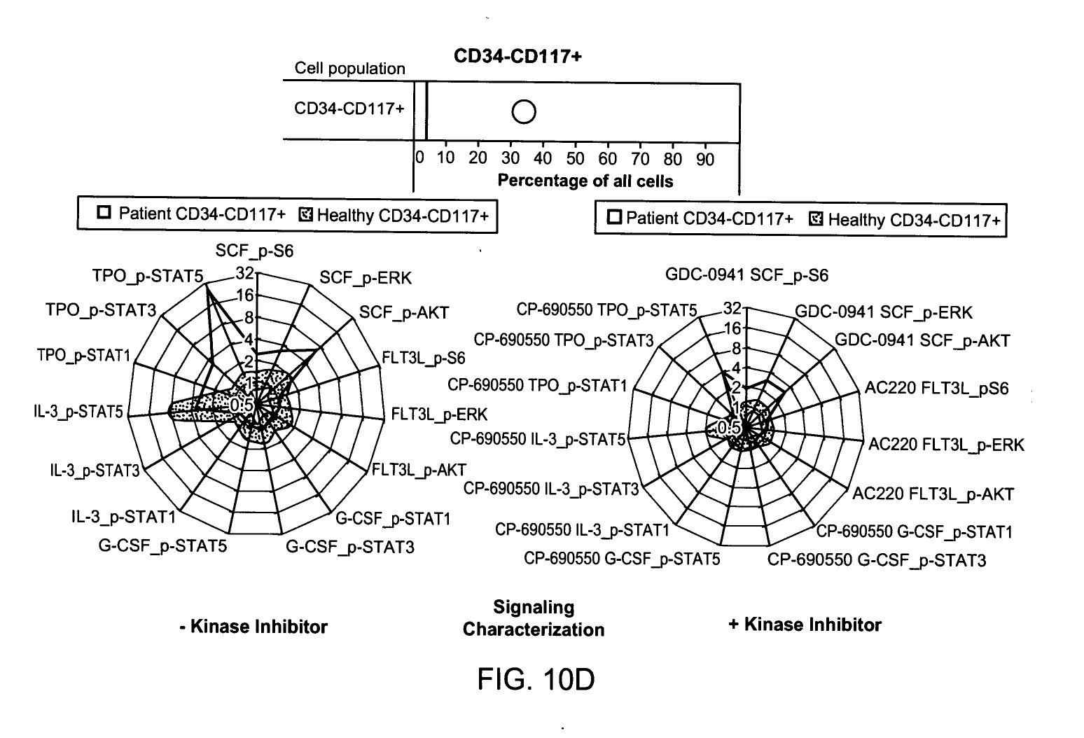

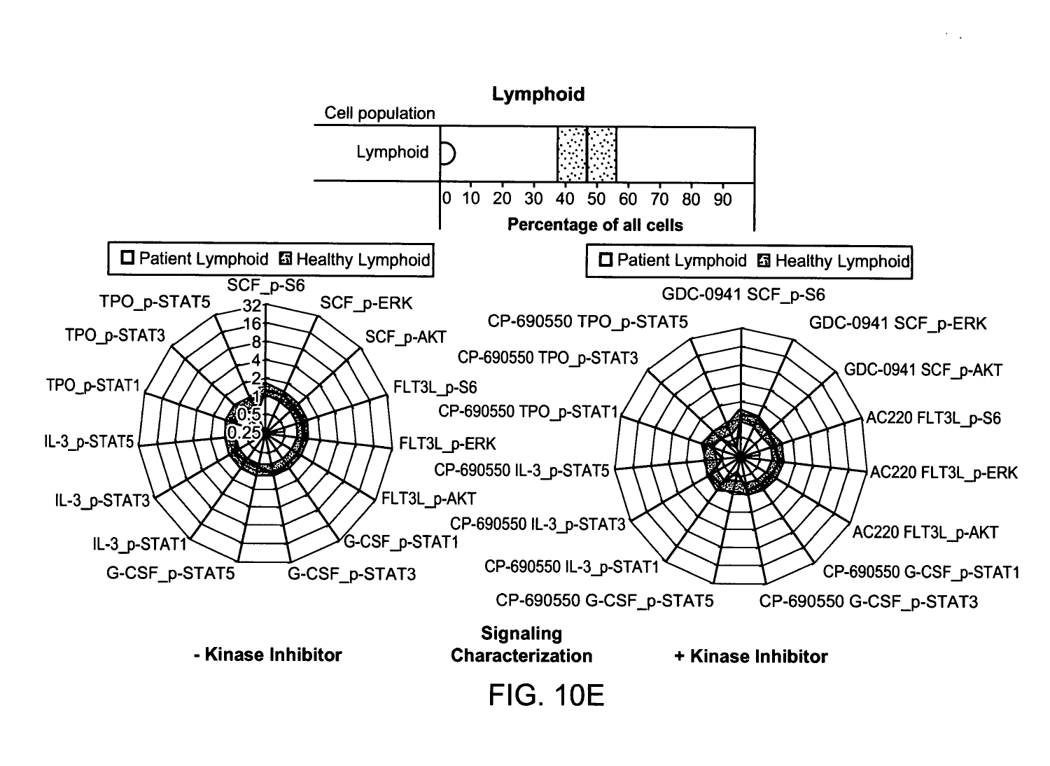

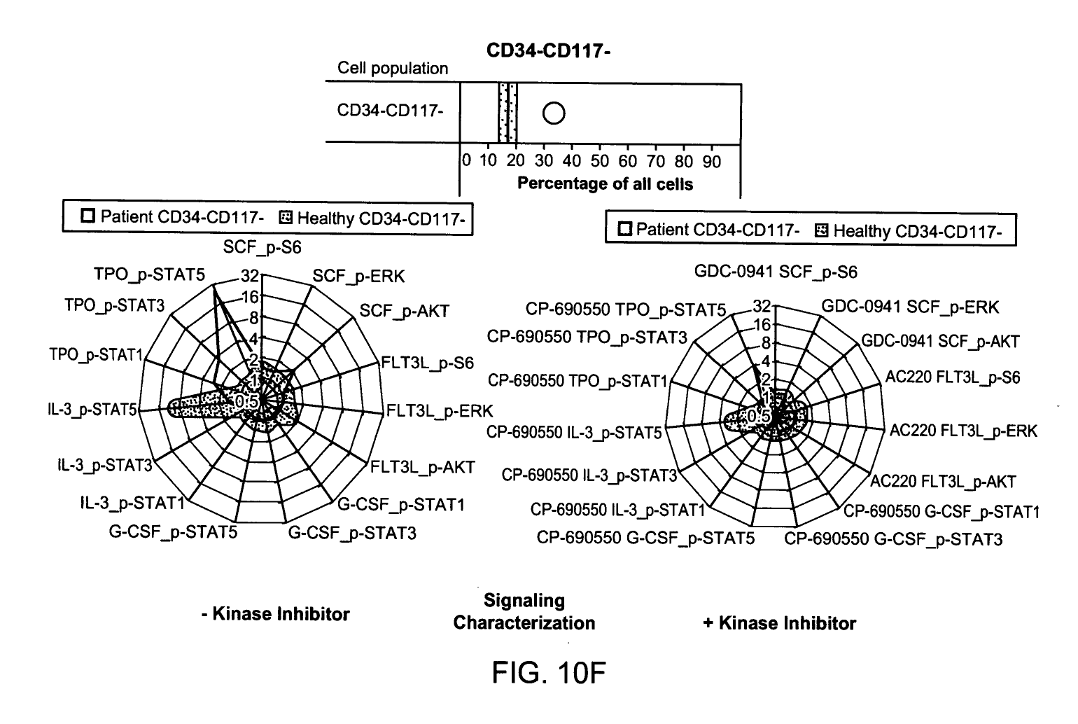

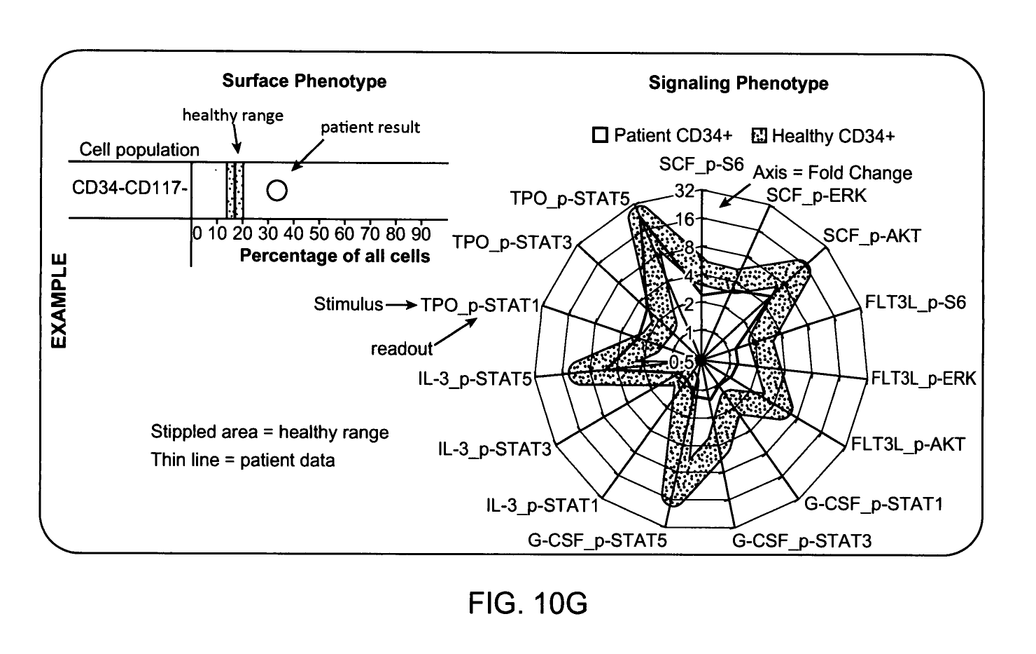

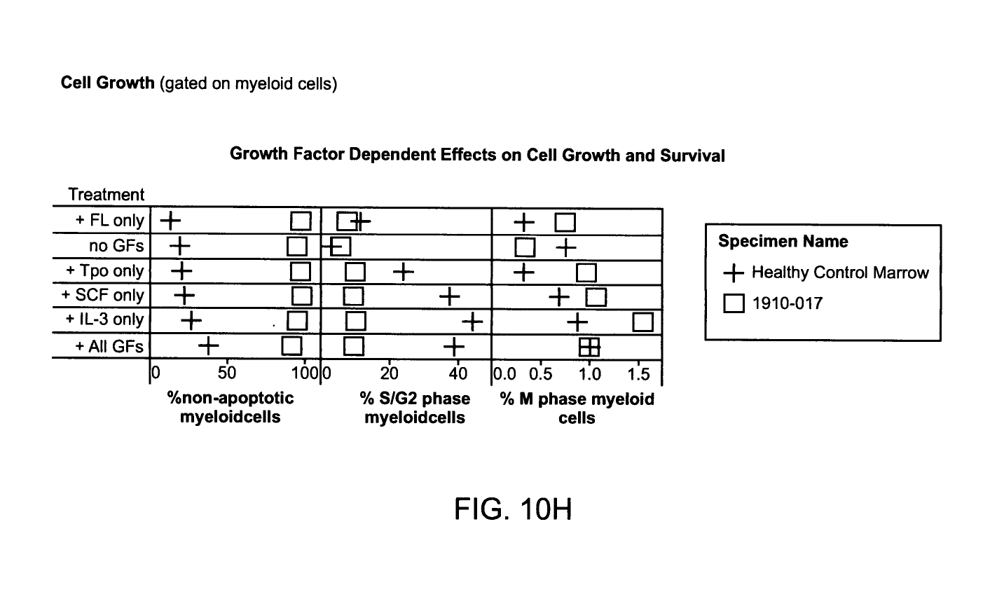

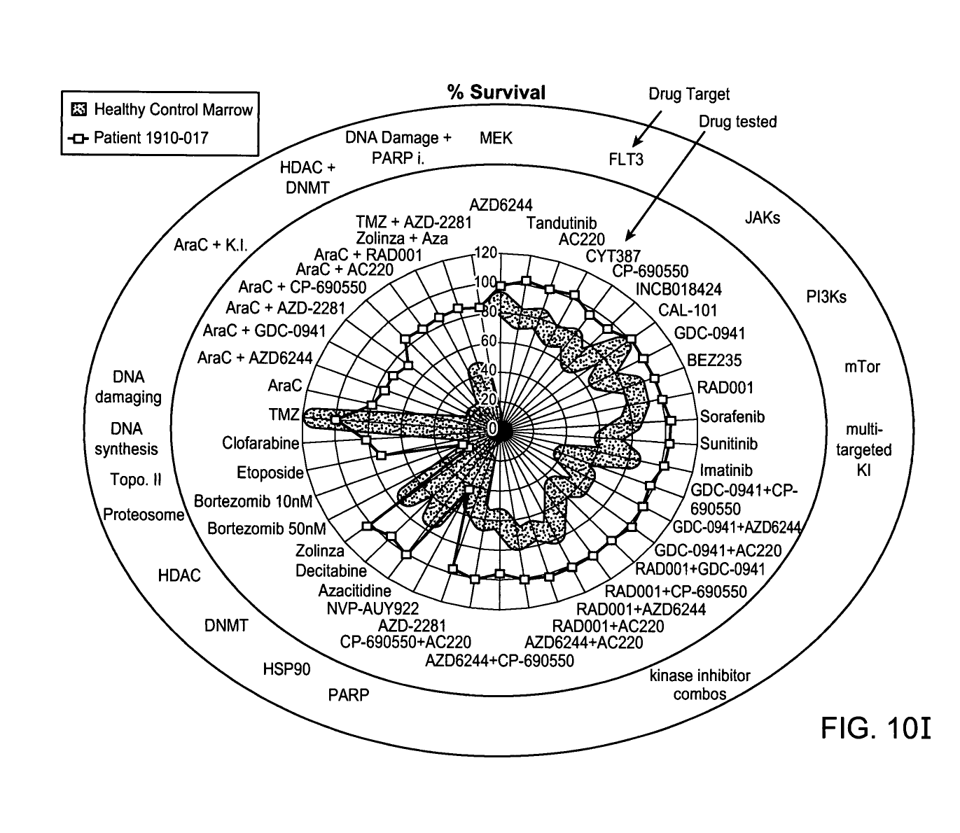

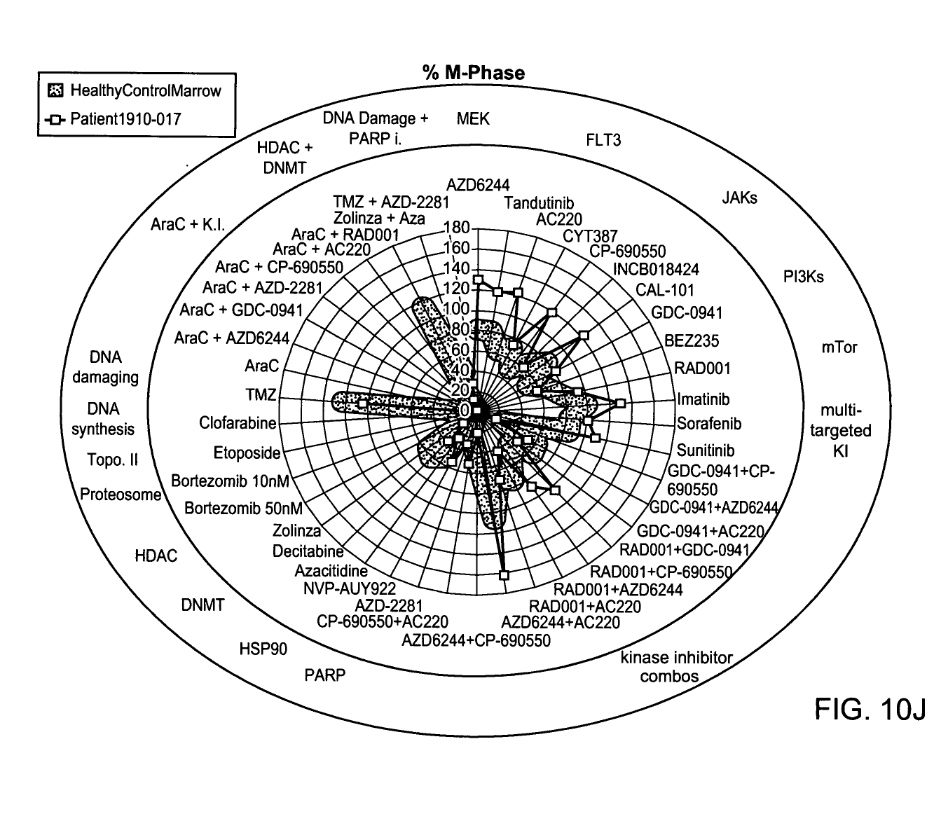

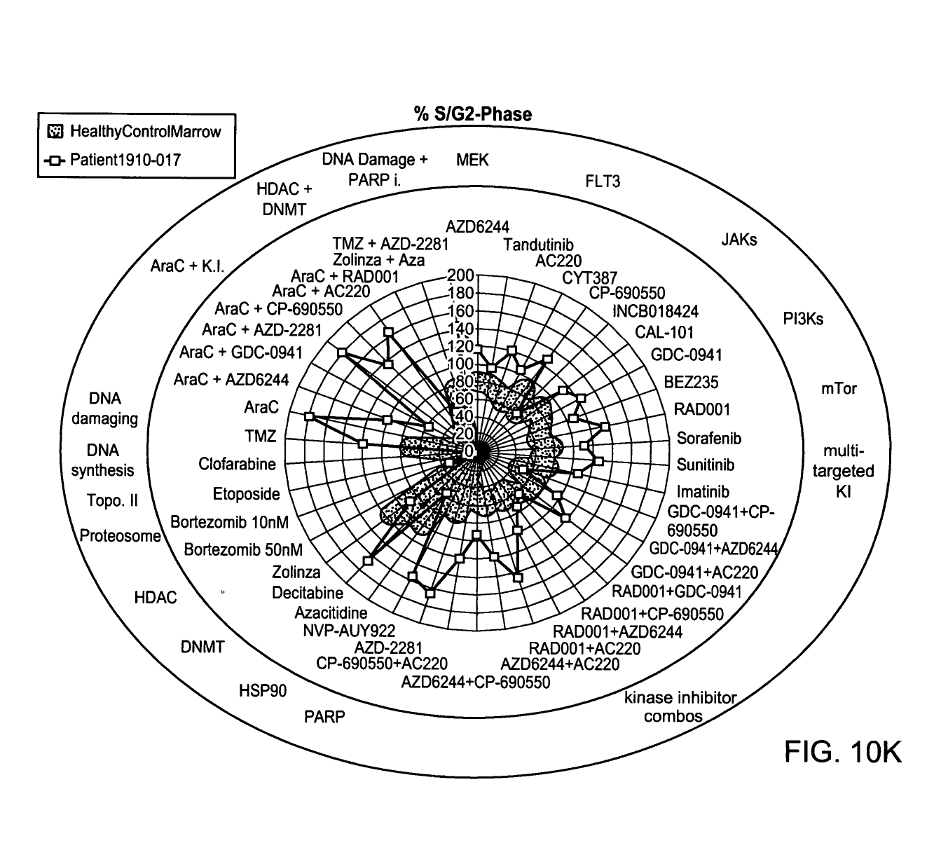

Examples of a report are shown in FIGS. 8, 9, and 10. A report can provide information on the types of cells in a patient sample (see e.g., FIGS. 8, 9, and 10). A report can comprise information on a percentage of a type of a cell in a patient sample (see, e.g., FIGS. 8, 9, and 10). A report can provide information on the percentage range of a type of cell in a normal or healthy sample. The type of cell can be determined based on the surface phenotype of the cell, and the surface phenotype of the cell can be included in the report. The range of percentage of normal or healthy cells in a sample can be compared to the percentage of a type of cell from a patient on a linear graph (see e.g., FIGS. 8 and 10) or a circular diagram (see e.g., FIG. 9A).

A report can provide information on a signaling phenotype. Signaling information can be presented as a radar plot (see e.g., FIGS. 8 and 10). A radar plot can also be known as a web chart, spider chart, star chart, star plot, cobweb chart, irregular polygon, polar chart, or kiviat diagram. Information on a report can include a comparison of signaling information from a patient (a test sample) to signaling information from normal or healthy samples. Information on normal samples can comprise information on the range of activation levels of activatable elements. The range can be indicated by a color, e.g., gray, on a radar plot. The range of activation levels can be expressed as fold changes in activation levels for activatable elements when cells are in the presence of a modulator relative to when cells are in the absence of the modulator. Other metrics can be used to compare patient samples to values for normal or healthy cells. The information on the activation levels of activatable elements from a patient (e.g., fold change when cells are in the presence of a modulator relative to cells in the absence of a modulator) can be plotted on the radar plot to allow a comparison of signaling data between the patient sample and the normal or healthy samples. Data on the patient sample can be represented in a different color than data for the normal or healthy samples, and different colors can be used for different cell subpopulations. A radar plot can include information on a modulator used in an experiment (e.g., TPO, SCF, FLT3L, G-CSF, IL-3) and on an activatable element (e.g., p-STAT3, p-ERK, p-AKT, p-S6, p-AKT, p-STAT1). The report can contain information regarding whether samples were treated or not treated with a kinase inhibitor. A report can illustrate cell lineage information (see e.g., FIG. 8).

Cell signaling information can also be represented as a heat map (see e.g., FIGS. 9B and 9C). The activation level of an activatable element relative to a basal state can be represented by a color scale. The color scale can comprise shades of yellow and blue or shades of red and green, for example.

A report can include information on cell growth (see e.g., FIGS. 9D and 10H). The information on cell growth can include information on one or more treatments, percentage of non-apoptotic cells, percentage of S/G2 phase cells, and percentage of M phase cells. The information on cell growth can compare cell growth of a patient sample to a normal or healthy control. The information on cell growth can include information on growth factor dependent effects on cell growth and/or survival.

A report can include information on the effects of a drug on a cell, e.g., cell survival and/or cytostasis (see e.g., FIGS. 9D, 9E, 10I, 10J, and 10K). Information on percent survival can be plotted as a radar plot, e.g., a survival radar plot (see e.g., FIG. 10I). The information on cell survival and/or cytostasis can include drug target and drugs that are tested. The percentage of non-apoptotic cells can be normalized to an untreated control (untreated can equal 100%). A color (e.g., gray) can show a range of response from a healthy sample, e.g., a healthy bone marrow sample. In the example shown in FIGS. 10J and 10K, for patient #1910-017, myeloid cells resisted apoptosis for most drugs, including AraC. However, two drugs were effective at inducing apoptosis: bortezomib (a proteosome inhibitor) and NVP-AuY922 (an HSP90 inhibitor).

Information on cell survival and/or cytostasis after drug exposure can include a cytostasis radar plot (see e.g., FIGS. 10J and 10K). As another example of information that can be included in a report, samples can be gated on non-apoptotic cell populations and that information can be displayed. A cytostasis radar plot can indicate cell-cycle information, e.g., a percentage of cells in M-phase or a percentage of cells in S/G2 phase normalized to an untreated control (e.g., an untreated control can equal 100%). In the examples shown in FIG. 10, although most drugs tested on patient sample #1910-017 have a mild effect on cell survival, many drugs can prevent cell growth (cytostasis). Information on apoptosis and cytostasis can be plotted as shown in FIGS. 9D and 9E. The results of other cell tests can be included in a report, such as those shown in U.S. Patent Publication No. 20100204973.

Direct graphical comparison between a range of activation level of an activatable element for normal or healthy cells compared to the activation level of the activation element for cells in a test sample (e.g., diseased cells) can identify aberrant signaling processes and/or survival mechanisms that can inform strategies for targeting a subject from whom the test sample was taken with a therapeutic. For example, aberrantly high thrombopoietin (TPO) signaling can reveal a dependence on TPO receptor signaling for optimal tumor cell survival and/or proliferation. Thus, targeting TPO signaling with one or more molecules that can attenuate the signal (e.g., kinase inhibitors, neutralizing antibodies, etc.) can slow tumor growth.

In some embodiments, a report can comprise information regarding, e.g., patient or subject indentifying information (e.g., name, age, gender, date of birth, weight, eye color, and/or hair color), insurance information, healthcare provider information (e.g., physician name, address of business, type of practice, etc.), medical history, blood pressure information, pulse rate information, information on therapeutics the subject is taking (e.g., name of therapeutic, dose, administration schedule, etc.), billing information, sample identification information, and/or order number. A report can comprise a summary, a diagnosis, a prognosis, or a therapeutic suggestion. A therapeutic suggestion can comprise a type of drug, a dose of drug, or a drug administration schedule. A report can comprise a barcode to identify the report or link the report to a subject. A report can comprise information on a clinical trial.

In some embodiments, a method is provided for determining an activation level of one or more activatable elements in normal cells and/or cells from a test subject (e.g., an undiagnosed subject), wherein the normal cells and/or cells from the test subject (e.g., an undiagnosed subject) are, or are not, contacted with a modulator, and transmitting data on the activation level of the one or more activatable elements to a central server for analysis and report generation. In one embodiment, a server communication module can receive a report from a central laboratory server. A report can comprise, e.g., a hyperlinked document, a graphic user interface, executable code, and/or physical document. A report can be accessed via a secure web portal. A server communication module can display a report to a third party and allow a third party to interactively browse a report. In some embodiments, a server communication module allows a third party to specify a format they would like to receive a report in or specific types of data (e.g., pathways data, clinical trials data, partner biometric data) they would like to include in a report. In an instance where a received report is associated with a patient sample, a server communication module can re-integrate patient information that has been scrubbed from clinical data in a report.

In one embodiment, a report generation module generates interactive reports which a third party can navigate to view report information. Reports can be displayed in a web browser or module software. A report generation module can generate a static report, e.g., a hard copy document.

A report generation module can function to generate a report for a third party based on the activation level of one or more activatable elements and an association metric. A report generation module can combine the activation level of one or more activatable elements and an association metric for a sample with additional information from public bioinformatics databases and partner a biometric information database to generate a report. A report generation module can retrieve data associated with a biological state from an external source such as a public bioinformatics database and combine this data with data on the activation state of an activatable element and an association metric to generate a report. In some embodiments, a report generation module can periodically retrieve this data and store the data in association with a statistical model in a biological state model dataset. A report generation module can retrieve clinical information associated with a sample from a partner biometric information database. A report generation module can also retrieve the activation level of one or more activation elements associated with a prior report for a client from an activation level database.

A report generation module can communicate with an activation level metric module, and a model generation module can generate graphical summaries of activation level data. Graphical summaries of data can include, e.g., bar plots of activation level data, gated plots of activation level data, line plots of activation level data, and pathway visualizations of activation level data. A report generation module can further communicate with an association metric module to produce a textual summary of association metric data. A textual summary can include a diagnostic of a disease state in a patient, recommended treatment regimen for a patient, a grade disease-subtype of a patient or a prognosis for a patient. A report generation module can incorporate graphical and textual summaries of activation level data into a report.

In some embodiments, a report generation module can then transmit a generated report to a third party client via a communication module or display a generated report to a third party client via a secure web portal. In other embodiments, a report generation module can physically transmit a report to a third party as a hard copy paper document or as executable code encoded on a computer-readable storage medium.

A report can be provided to a subject (e.g., a subject from whom a test sample was taken). A report can be provided to an insurance company. A report can be provided to a healthcare provider (e.g., physician, surgeon, nurse, first responder, dentist, psychiatrist, psychologist, anesthesiologist, etc.).

### Sample Grouping or Characteristic

In some embodiments, samples from a test subject, e.g., an undiagnosed individual (e.g., samples comprising undiagnosed cells) and normal individual (normal cells) can be compared based on a sample grouping or characteristic, e.g., age, race, gender, ethnicity, physical characteristic, socioeconomic status, income, occupation, geographic location of birth, education level, diet, exercise level, etc.

A sample grouping or characteristic can be age. The age of an individual (e.g., test subject or, normal subject) from whom a sample can be derived can be about, more than about, or less than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46. 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119 or 120 years old. The test subject (e.g., undiagnosed individual) or normal subject can be, e.g., a fetus, a newborn, an infant, a child, a teenager, an adult, or an elderly person. An activation level of one or more activatable elements in an a sample from a test subject (e.g., an undiagnosed sample; sample from an undiagnosed individual) can be compared to an activation level of the one or more activatable elements from normal samples derived from normal subjects that are, e.g., about 1-5, 5-10, 1-10, 10-15, 10-20, 15-20, 20-25, 20-30, 25-30, 30-35, 35-40, 40-45, 40-50, 45-50, 50-55, 50-60, 55-60, 60-65, 60-70, 65-70, 70-75, 75-80, 70-80, 80-85, 80-90, 85-90, 90-95, 90-100, 95-100, 100-105, 100-110, 105-110, 110-115, 110-120, 115-120, 1-20, 20-40, 40-60, 60-80, 80-100, or 100-120 years old. A test subject can be of an age that falls into any one of the aforementioned ranges. A test subject and/or normal subject can be about, more than about, or less than about 1, 2, 3, 4, 5, 6, 7, 8, 9, 10, 11, or 12 months old. Normal subjects can be selected for analysis based on the age of the normal subjects.

A sample grouping or characteristic can be race, ethnicity, birth country, and/or geographic location. A sample grouping or characteristic of a test subject and/or normal subject can be, e.g., a European American, an African-American, Caucasian, Asian, Hispanic, or Latino. In another embodiment, a sample grouping or characteristic of a test subject and/or normal subject can be, e.g., Abzinz, Abenaki, Abipones, Abkhazs, Aborigines, Abron, Acadian, Accohannock, Achang, Acelmese, Acholi, Achomawi, Acoma, Adi, Adjarians, Adyghe, Adyhaffe, Aeta, Afar, African-American, African Canadian, African Hebrew Israelites of Jerusalem, Afrikaners, Afro-American peoples of the Americas (e.g., Afro Argentine, Afro Bolivian, Afro Brazilian, Afro-Chilean, Afro-Colombian, Afro-Costa Rican, Afro-Cuban, Afro-Dominican, Afro-Ecuadorian people, Afro-Guyanese, Afro-Latino, Afro-Jamaican, Afro-Mexican, Afro-Peruvian, Afro-Portuguese, Afro-Puerto Rican, Afro-Trimidadian, Afro-Uruguayan), Aftsarians or Isaurians, Agaw, Agni, Aguls, Ahtna, Aimaq, Ainu, Aynu of China, Aja, Aka, Akie, Ak Chin, Akan, Akha, Akuapem, Akhvakh people, Akyem, Alabama, Alak, Albanians, Albanian American, Albanian Australian, Aleut, Algonquian, Aliutors, Alsatians, Amahuaca, Amerasians, Americo-Liberians, Amhara, Amish, Amungme, Andalusians, Andis, Anga, Anglo-African, Anglo-Celtic Australian, Anglo-Indian, Anglo-Saxon, Annamites or Vietnamese or Kinh or Jing, Ansar people or Ansarie, Anuak, Apaches, Apinaje, Arab (e.g., Palestinian diaspora, Afro-Arab, Arab American, Arab Argentine, Arab Australian, Arab Brazilian, Arab Britons, Arabs in Bulgaria, Arab Canadian, Arab Chilean, Arab Ecuadorians, Arab Haitian, Arabs in France, Arabs in Germany, Arabs in Greece, Arabs in Palestine, Arabs in Italy, Arab Jews, Arab Mexican, Arabs in the Netherlands, Arabs in Pakistan, Arab Peruvian, Arab Singaporean, Arab Sri Lankans, Arabs in Sweden, Arabs in Turkey, Arab Venezuelan, Arab diaspora in Colombia, Arabs in Afghanistan, Iranian Arabs), Aramaic, Araon, Aragonese, Arapaho, Arawak, Arbëreshë, Archis, Arikara, Armenians, Armenian American, Aromanians (or Macedo-Romanians), Arvanites, Atoni, Aryans/Indo-Iranians, Indo-Aryan peoples, Iranian peoples, Nuristani people, Asante, Asheninka, Asmat, Assiniboine, Assyrians, Asturians, Atacameno, Atta, Ati, Atikamekw, Atsina, Atsugewi, Aukstaitians, Australian aborigine, Austrians, Avars, Awá, Aymaras, Ayta, Ayrums, Azeris, Aztecs, Ayapaneco, Babongo, Bahrani people, Badui, Baggara or Baqqarah, Baguirmi, Bagulals, Bai, Bajau, Baka, Bakhtiyari, Balinese, Bakongo/Kongo, Balkars, Baloch (also Baluch, Balochi), Baltic Germans, Bamar (also Burmese and Burman), Bambara, Bamileke, Banat Swabians, Banawa, Banda, Bandjabi, Banjar, Bantu, Baoule, Bapou, Bariba, Bartangs, Basarwa, Bashkirs, Basotho, Basques, Basque Argentine, Basque American, Basque Chilean, Bassa, Bassari, Baster (also known as Baaster), Batak, Batak, Bateke, Bats, Batswana (also Tswana), Bavarians, Beaver, Bedouins, Beja, Belarusians, Bengalis, Bengali American, Bengali Hindus, Bemba, Bene Israel, Berbers, Berom, Betamaribe, Bethio, Beti-Pahuin, Bezhtas, Bhotia, Bhotiya, Bicolano, Biharis, Blackfeet (or Blackfoot), Black British, Black Canadians, Black Indians, Bo Y, Bodo, Boere-Afrikaners, Bonairean, Bonan, Borinquen, Bosniaks, Bostonian, Botlikhs, Bouganvilleans, Boyar, Boyko, Bozo, Brau, Brazilian, Bretons, British, British American, British Canadian, British Chileans, Brulé, Bru-Van Kieu, Bubi, Budukhs, Bugis, Bulang, Bulgarians, Bulgars, Bunjevci, Burgenland Croats, Buryats, Bushongo, Buyi, Caddo, Cahuilla, Caingang, Cajun, Caldoche, Californio, Cambodia, Campa, Canadians, Canarians, Cantonese, Cape Coloured, Cape Malay, Castilians, Caprivian, Caribs, Carinthian Slovenes, Caripuna, Catalans, Catawba, Cayuga, Cayuse, Cebuano, Celts, Ceylon Moors, Chagga, Cham, Chambri, Chamalals, Chamorro, Charrúa, Chechens, Chehalis, Chemehuevi, Chepang, Chere, Cherokee, Cheyenne, Chicanos, Chickahominy, Chickasaw, Chilcotin, Chileans, Chilean American, Chilean Australian, Chilean Swedes, Chimakum, Chinese (also known as Han or Han Chinese), Chinese American, Chinese Australian, Chinese Brazilian, Chinese Canadian, British Chinese, Ethnic Chinese in Brunei, Chinese people in Bulgaria, Burmese Chinese, Chinese Cambodian, Chinese Canadian, Chinese people in Chile, Chinese-Costa Rican, Chinese Cuban, Chinese in Fiji, Chinese Filipino, Chinese diaspora in France, Chinese Indonesian, Chinese people in Italy, Chinese Jamaican, Chinese people in Japan, Ethnic Chinese in Korea, Laotian Chinese, Malaysian Chinese, Chinese Mauritian, Chinese Mexican, Ethnic Chinese in Mongolia, Chinese New Zealander, Chinese Nicaraguan, Ethnic Chinese in Panama, Chinese Peruvian, Chinese of Romania, Ethnic Chinese in Russia, Chinese in Samoa, Chinese Singaporean, Chinese South Africans, Chinese people in Spain, That Chinese, Chinese in Tonga, Chinese Trimidadian, Chinese Vietnamese, Chinookan, Chipewyan, Chippewa, Chitimacha, Cho Ro, Choctaw, Chukchansi, Chukchis, Chulym Tatars, Chumash, Chuncho, Chut, Chuukese, Chuvash, Ciboney, Circassians or Cherkezians, Clayoquot, Co people, Coalhuiltec, Co Ho people, Co Lao, Co Tu people, Coast Salish, Cochiti, Cocopah, Coeur d'Alene, Coharie, Colchians or Kolchians, Colombians, Coloured, Colville, Comanche, Comorian, Cong, Congolese people, Copper, Coquille, Corsicans, Cornish, Cornish American, Cornish Australian, Cossack, Costanoan, Coushatta, Cowichan, Cowlitz, Cree, Creek, Créole, Crimean Germans, Crimean Goths, Crimean Tatars, Croats, Croatian American, Croatian Australian, Croatian Brazilian, Croatian Canadians, Croatian Chileans and Croatian-Peruvians, Crow, Cubans, Cuban Americans, Cumans, Cupeño, Curaçaoan, Greek Cypriots, Czechs, Czech American, Czechs in the United Kingdom, Czech Canadian, Daasanach, Dadhich, Dai (That, That Lue), Dakelh, Dakota, Damara, Danish, Danish American, Danish Australian, Danish Canadian, Danmin, Darhad, Dargins, Daribi, Daur, Dayaks, De'ang, Deg Hit'an, Degar (Montagnards), Delaware, Dena'in a (also known as the Tanaina), Dendi, Derbish, Desana, Dhivehis, Dhodia, Didos, also known as Tsez, Diegueno, Dinka, Diola, Dogon, Dolgans, Dom, Doma, Dominicans, Dominican American, Don Cossacks, Dong, Dongxiang, Dorze, Dorians, Dravidians, Drung, Druze, Du people, Duala people, Dungan, Dutch, Dutch American, Dutch Australian, Dutch Brazilian, Dutch Canadian, Cape Dutch, Dutch New Zealander, Dyula (Jula), Ebira, Ecuadorian, Egyptians, Elema, Enets, Enga, English, English American, English Australian, English Canadian, English Brazilian, English African, English Argentine, Anglo-Burmese, Anglo-Indian, Enxet, Eshira, Eskimo, Esselen, Estonians, European Americans, Evens, Evenki, Ewe, Expatriata Americana, Falasha/Beta Israel, Fante, Faroese, Fars, Fereydan, Fernandinos, Fijian, Fir Bolg, Finns, Finnish American, Flemish, West Flemings, Fon, Fox, Franco-Mauritian, Franco-Réunionnaise, Franks, Franconians, French, French American, French British, French Mexican, French Argentine, French Chilean, French Canadian, Frisians, Fula (also called Fulani or Fulbe), Fulni-o, Fur, Ga, Gaels, Gagauz, Galicians, Gaoshan, Garifuna/Garinagu, Garo, Gbaya people, Ge, Geba Buru, Gelao, Georgian, Georgian American, Germans, German American, German Argentine, German Australian, German Brazilian, German-Briton, Germans in Bulgaria, German Canadian, German-Chilean, Germans in the Czech Republic, Germans of Hungary, Germans of Kazakhstan, German Mexican, German Peruvian, Germans in Poland, Germans in Romania, Germans from Slovakia, Germans of Yugoslavia, Gia Rai, Giay, Gie Trieng, Gitanos, Godoberis, Gogodali, Gongduk, Gorals, Gorani, Goshute, Gotlanders, Goulaye, Greeks, Griqua, Gros Ventre, Gruzinim, Guadeloupean, Guajajara, Guarani, Gujaratis, Gullah, Gurage, Guria, Guru, Guruks, Gurung, Hadza, Haida, Haitian Creole, Hakka, Haliwa-Saponi, Hamer, Hamshenis, Han Chinese, Hani, Hausa, Havasupai, Haw, Hawaiian, Hapas, Hazara, Herero, Hesquiat, Hezhen, Hidatsa, Himba, Hindoestanen, Hindi people, Hinukhs, Hispanics, Hmar, Hmong, Hoa, Ho-Chunk, Hoh, Hohokam, Hoklo, Holikachuk, Hopi, Houma, H'Re, Hualapai, Huastec, Hui Chinese, Huicol, Hungarians, Huns, Hunzakuts, Huli, Hunzibs, Hupa, Hurrians, Huron, Hutsuls, Hutu, Iatmul, Iban, Ibanag, Ibibio, Icelanders, Icelandic American, Icelandic Canadian, Igbo, Igbo American, Igbo Jamaican, Igbo Canadian, Igorot, I-Kiribati, Illiniwek, Ilocano, Ilonggo, Imereti, Incan, Indo-Aryan, Indo-Caribbean, Indo-Europeans, Indo-Guyanese, Indo-Iranians, Indo-Aryan peoples, Iranian peoples, Nuristani people, Indo-Trimidadian, Ingessana, Ingrians, Ingushes, Innu, Inuit, Irani, Iranian, Irish, Irish American, Irish Argentine, Irish Australian, Irish British, Irish Canadians, Irish Chilean, Irish Puerto Rican, Irish Mexican, Irish Newfoundlanders, Irish Traveller, Irish Quebecers, Iroquois, Ishkashmis, Isleta, Isoko, Istriot, Istro-Romanians, Italians, Italian American, Italian Argentine, Italian Australian, Italian Brazilian, Italians in the United Kingdom, Italian Canadian, Italian-Chilean, Italian Egyptian, Italians in Germany, Italian Jews, Italian settlers in Libya, Italian Mexican, Italian Peruvian, Italians of Romania, Italian Scots, Italian Swiss, Tunisian Italians, Italian settlement in Uruguay, Italo-Venezuelans, Welsh Italians, Itelmens, Itsekiri, Izhorians, Jakaltek people, Jakut, Jamaican, Janjevci, Japanese, ethnic Japanese, Japanese Americans, Japanese Australians, Japanese Brazilians, Japanese Canadians, Japanese Chileans, Japanese settlement in the Philippines, Japanese people in France, Japanese people in Germany, Japanese in Hawaii, Japanese Mexicans, Japanese in the United Kingdom, Japanese Peruvians, Jassic (Jász), Jat, Javanese, Jebala, Jemez, Jewish, Jing, Jingpo, Jino, Jivaro, Jola, Jopadhola, Jri, Jutes, Kandahar and Kabuli, K'iche' (Quiché), Kabardin, Kabyle, Kadiweu, Kaibartta, Kakheti, Kalasha of Chitral, Kalenjin, Kallawaya, Kaliai, Kalispel, Kabuli, Kamas, Kamayura, Kannadiga, Kanembu, Kapauku, Kapampangan, Karachay, Karaims, Karajá, Karakalpaks, Karamanlides, Karamojong, Karatas, Karelians, Karen, Karok, Kashubians, Katang, Kato, Katuquina, Kavango, Kaw or Kansa Indians, Kayapo, Kazakhs, Kenyah, Kenyan American, Kereks, Keresan, Kets, Khakas, Khang, Khants, Khasia, Khassonké, Khevi, Khevsureti, Khinalugs, Khmer, Khmer American, Khmu, Kho Mu, Khoikhoi, Khojas, Khomani or Nu, Khufis, Khvarchis, Kickapoo, Kikuyu, Kinh or Jing or Vietnamese, Kiowa, Klallam, Klamath, Klikitat, Kolchan, Kombai, Kogi, Komi, Koniag, Kongo, Kootenai, Koptian, Korean, Korean American, Koreans in Argentina, Korean Australian, Korean Brazilian, Koreans in the United Kingdom, Korean Canadian, Koreans in Chile, Koreans in China, Koreans in the Philippines, Koreans in France, Koreans in Germany, Koreans in Guatemala, Koreans in Hong Kong, Koreans in the Arab world, Koreans in Indonesia, Koreans in Iran, Koreans in Japan, Koreans in Malaysia, Korean Mexican, Koreans in Micronesia, Korean New Zealander, Koreans in Paraguay, Koreans in Peru, Koreans in Poland, Koreans in Singapore, Koreans in Taiwan, Koreans in Uruguay, Koreans in Vietnam, Korean adoptees, Korowai, Koryaks, Kosraean, Koskimo, Koyukon, Kpelle, Kraho, Krashovans, Kri, Krymchaks, Kryz, Kuban Cossacks, Kubu, Kuikuru, Kuna, Kumeyaay, Kumyks, Kurds, Kuruba Gowda, Ktunaxa, Kwakiutl, Kwakwaka'wakw, Kyrgyz, La Chi, La Ha, La Hu, Laguna, Lahu, Laigain, Lakota, Laks, Lamet, Langi (also Lango), Lao, Lao American, Lao Sung, Lao Theung, Latgalians, Latvians, Lavae, Laven, Layap, Laz, Lazoi, Lebanese people, Lebanese American, Lebanese Australian, Lebanese Brazilian, Lebou, Lemkos, Lenca, Lengua, Leonese, Lezgis, Lhoba, Lhotshampa, Li, Liechtenstein, Limbus, Lipka Tatars, Lipovans, Lisu, Lithuanians, Livonians, Lo Lo, Lobi, Lotuko, Louisiana Creole people, Lozi, Lua, Luba, Lue, Luhya, Luiseno, Lumad, Lumbee, Lummi, Lunda, Luo (also Joluo), Lusitanians, Luso-Brazilians, Luso-American, Luxembourgers, Luxembourg American, Maasai, Macao, Macedonians, Macuxi, Madeirans, Madurese, Magar people, Magyars, Magyar American/Hungarian American, Magyar Canadian/Hungarian Canadian, Magyar Vojvodinian/Hungarians in Vojvodina, Mahican, Mahorian, Maidu, Mailu, Maingtha, Maka, Makah, Makong, Makua, Malagasi, Malay, Malayalee, Maliseet, Maltese, Mam, Mamamwa, Manasi, Manchu, Mandan, Mandinka, Mang people, Mangbetu, Mangyan, Mansis, Manx, Maonan, Māori, Mapuche, Maratha, Marathis, Mari, Maricopa, Marind-Anim, Mashantucket Pequots, Matabele, Mataco, Matis, Mattaponi, Maubere, Maya, Mayo, Mazandarenis, M'Baka, Mbaya, Mbochi, Mbuti, Megleno-Romanians, Meherrin, Mekeo, Melungeons, Memon, Menba, Mende, Menominee, Mennonites, Amish or the Pennsylvania Dutch, Hutterites, Mentawai, Meskhetians, Mestizo, Métis, Meitei, Me-Wuk, Mbuti, Miccosukee, Mi'kmaq, Mina, Mekeo, Mexican people, Minahasa/Manadonese, Minangkabau, Mingo, Mingrelians, Miskito, Mission, Mitsogo, Miwok, Mixtec, Mizo, Mlabri, Mnong, Modoc, Mohajir, Mohave, Mohawk, Mohegan, Molise Croats, Mon, Monacan, Mongo, Mongols, Mono, Montagnais, Montaukett, Montenegrins, Moor, Moravians, Moriori, Morisco, Morlachs, Mormons, Moro people, Mossi, Motuan, Muckleshoot Indians, Mudéjar, Muhajir (Pakistan), Mulam, Mulatto, Mundas, Mundurucu, Muong, Mursi, Museu, Myene, Naga, Nahanni, Nahua, Namaqua, Nanais, Nansemond, Narragansett, Nasia, Natchez, Nauruan, Navajo, Naxi, Ndau, Ndebele, Negidals, Negrito, Nenets, Nespelem (Nespelim or Nespilim), Nevisian, Newar, Nez Percé, Ngac'ang, Ngasan, Ngae, Nganasans, Nhahuen, Nhuon, Niominka, Nipmuc, Nishka, Nisqually, Nisei, Nisse, Nivkh, Niuean, Ni-Vanuatu, Njem, Nogais, Nomlaki, Nooksack, Norwegians, Norwegian American, Norwegian Canadian, Nu, N/u or Khomani, Nuba, Nubians, Nuer, Nukak, Nung, Nuristani, Nuu-chah-nulth, Nyagatom, Nzema, O Du, Odawa, Ogaden, Oglala, Ogoni, Ojibwa, Okamba, Okande, Okinawans, Omaha, Omagua, Oneida, Onondaga, O'Odham, Oroch, Orokaiva, Oroks, Oromo, Orogen, Oroshoris, Osage Nation of Oklahoma (or of Missouri, Kansas, and Arkansas), Ossetians, Otavalerio, Otoe-Missouria, Ottawa, Ovambo, Pa Then, Paiute, Pākehā, Pakoh, Palcene, Paliyan, Pamunkey, Pangasinan people, Panoan, Pa-O, Pashu, Pashtun (Pathan), Parsi, Passamaquoddy, Pataxo, Pattar, Pa-Thi, Paugusset, Pawnee, Pennsylvania Dutch, Penan, Pennsylvania German, Penobscot, Peoria, Perce, Persians, Petchenegs, Phoenicians, Phong, Phu La, Phu Noi, Phu That, Picts, Pied-noir, Piegan, Pima, Pit River Indians, Pitcairn-Norfolk, Pilaga, Polabian Slays, Polish, Polish American, Polish Australian, Polish Argentine, Polish Brazilian, Poles in the United Kingdom, Polish Canadian, Poles in Germany, Polish minority in Ireland, Polish minority in Russia, Poles in Belarus, Poles in Czechoslovakia, Poles in Ireland, Poles in Latvia, Poles in Lithuania, Poles in Romania, Poles in the former Soviet Union, Poles in the Soviet Union, Poles in Ukraine, Polynesians, Pomaks, Porno, Ponca, Ponhpeian, Pontic Greeks, Poospatuck, Portuguese, Portuguese Brazilian, Portuguese American, Portuguese Canadian, Potawatomi, Potiguara, Powhatan, Proto-Indo-Europeans, Pu Peo, Pueblo people, Puelche, Puerto Ricans, Puerto Ricans in the United States, Puget Sound Salish, Purépecha, Punan, Pumi, Punjabis, Puyallup, Qashqai, Q'eros, Qiang, Quahatika, Quapaw, Quechan, Québécois, Quechuas, Quiché (K'iche'), Quileute, Quinault, Quinqui, Ra Glai, Rais, Rakhine, Rakuba, Ramapough Mountain Indians, Rappahannock, Rashaida, Ro Mam, Rohingya, Roma, Romanians, Romanian American, Roshanis, Rotuman, Russians, Russian American, Russian Australian, Russians in Belarus, Russians in Bulgaria, Russian Brazilian, Russian Canadian, Russians in China, Russians in Finland, Russians in Georgia, Russians in Japan, Russians in Kazakhstan, Russians in Korea, Russians in Ukraine, Russians in Mexico, Russians in Germany, Rusyns, Ruthenians, Rutuls, Ryukyuans, Sadang, Saek, Saho, Saingolo, Salar, Salish, Samanthan, Samaritan, Samegrelo, Sami, Samoans, Samogitians, Samojeeds, Samtao, Samburu, San, San Chay, San Diu, Sanema, Santal, Santee Sioux, Saponi, Sara, Saramaka, Sarakatsani, Sardinians, Sauk, Sauk-Suiattle, Saxons, Scottish-American, Scots-Irish, or Scotch-Irish, Scots-Irish American, Scottish, Sekani, Selk'nam, Selkies, Selkups, Semai, Seminole, Sena, Seneca, Senegalese people, Sentinelese, Serbs, Serer, Serer-Ndut, Seychellois Creole people, Seychellois people, Shan, Shangaan, Shasta, Shavante, Shawnee, She, Sherpa, Shinnecock, Shipibo, Shoalwater Bay Tribe, Shona, Shors, Shoshone, Shughnis, Shui, Si La, Sicilians, Sicilian American, Sidamo, Siddi, Siksika, Silesians, Siletz, Sindhis, Singmun, Sinhalese or Sinhalas, Sinti, Sioux, Siuslaw, Skagit, S'Klallam, Skokomish, Skwxwú7mesh, Slays, Slovaks, Slovak American, Slovaks in Bulgaria, Slovaks in Vojvodina, Slovenes, Slovene Americans, Slovene Australians, Slovene Canadians, Slovene Hungarians, Sokci, Somali, Somba, Songhai, Soninke, Sorbs, Souei, (South African), Southern Tutchone, Southerners or Southern Americans, Spanish, Spanish American, Spokane, Squaxin Island Tribe, Sri Lankan Moors, Stillaguamish, Sundanese, Sudanese people, Sudanese American, Sudanese Australian, Suquamish, Suri, Surui, Susu, Suyá, Svans, Aramean-Syriacs, Swahili people, Swazi, Swedes, Swedish American, Swedish Argentine, Swedish Australian, Swedish British, Swedish Canadian, Swedish Estonian, Swedish Finns, Swinomish, Swiss, Swiss German, Swiss French, Swiss Italian, Swiss Romansh, T'boli, Ta Oi, Tabasarans, Tache, Tachi, Tagalogs, Tagish, Taíno, Taiwan, Taiwanese American, Taiwanese aborigines, Tajik, Tajiks in China, Taliang, Talysh, Tamang, Tamil, Tamil British, Tamil Canadian, Tamil Indians in Sri Lanka, Tamil Malaysians, Tamil Sri Lankans, Tanna, Tanana, Taos, Tapajo, Tapirapé, Tapuia, Tarahumara, Tarascan, Tasaday, Tatars, Tats, Tay, Teda, Tehuelche, Teimani Jewish, Tejano, Telefolmin, Terena, Tetons, Tewa, Texans, That, That American, That Australian, That British, Thakali, Tharu, Thin, Th{circumflex over (\dot{o}, Tibetans, Ticuna, Tigray people, Tigray-Tigrinia, Tigre people, Tigrinya people, Tigua, Tindis, Tipra, Tlakluit, Tlingit, Toala, Toba, Tofalars, Tohono O'odham, Tokelauan, Tolowa, Tolais, Toltec, Tonga, Tongans, Tongva, Tonkawa, Topachula, Toraja, Torbesh, Torres Strait Islanders, Totonac, Toubou, Transylvanian Saxons, Trukhmens, Tsakhurs, Tsetsaut, Tsez, Tsimishian, Tsonga, Tsuu T'ina, Tswana people, Tuareg, Tujia, Tukano, Tukolor, Tuamotu, Tulalip, Tulutni, Turn, Tumbuka, Tungus, Tunica-Biloxi, Tupian, Tupinamba, Turkmen, Turks, Turkish American, Turkish Australian, Turks in Austria, Turks in Azerbaijan, Turks in Belgium, Turkish British, Turkish Canadian, Turkish Cypriots, Turks in Denmark, Turks in France, Turkish Germans, Turks in Japan, Turks in Liechtenstein, Turks in the Netherlands, Turks in Norway, Turks in Sweden, Turks in Switzerland, Tusheti, Tutsi, Tuvaluans, Tuvans, Twa peoples, Txicao, Tzigane, U'wa, Ubykh, Udeghes, Udis, Ukrainian, Ukrainian American, Ukrainian Argentine, Ukrainian Canadian, Ukrainian Russian, Ulchs, Ulster-Scots, Ulta, Umatilla, Umpqua, Upper Skagit, Urapmin, Ute, Uyghur, Uzbek, Valencian people, Vaturanga, Venda, Venetians, Veps, Vietnamese or Kinh or Jing or archaically Annamites, Vietnamese American, Vietnamese people in the United Kingdom, Vietnamese people in the Czech Republic, Vietnamese Norwegians, Vietnamese people in Bulgaria, Vietnamese people in Russia, Visayan, Vlachs, Volga Germans, Votes, Wa, Wabanaki, Waccamaw, Wailaki, Waitaha, Waiwai, Waki, Wakhs, Walla Walla, Walsers, Wampanoag, Wasco, Washoe, Wayana, Welayta people, Welsh, Welsh American, Welsh Australian, Welsh Canadian, Wends, White Mountain Apache, Wichita, Wintun, Wiyot, Wolof, Wu Chinese, Wyandot, Wyyanaha, Xakriabá, Xavante, Xerente, Xhosa, Xibe, Xikrin, Xin Uygurs, Xinh Mun, Xo Dang, Xtieng, Xucuru, Xueda, Yae, Yaghan, Yaghnabis, Yagua, Yakama or Yakimas, Yakughir, Yakuts, Yang, Yankton Sioux, Yanomami, Yao, Yavapai: Yavapai-Apache Nation, Yavapai-Prescott Indian Tribe, Yapese, Yaqui, Yawanawa, Yawalpiti, Yazgulamis, Yekuana, Yi, Yocha-Dehe, Yokut, Yoruba, Yörük, Yuchi, Yugur, Yukaghirs, Yuki, Yuma, Yumbri, Yupik, Yurok, Yu people, Zaghawa, Zambo, Latino Zamboangueño, Zapotec, Zarma, Zeibeks, Zazas, Zhuang, Zou, Zulian, Zulu, or Zuni.

A sample grouping or characteristic can be gender. Gender can be male or female.

A sample grouping or characteristic can be socioeconomic status. Socioeconomic status can comprise, e.g., low, middle, or high. Socioeconomic status can be based on income, wealth, education, and/or occupation.

A sample grouping or characteristic can be highest education level of a subject. Education level can be, e.g., kindergarten, primary (e.g., elementary) school, middle school, secondary school (e.g., high school), college or university, junior college, graduate school, law school, medical school, or technical school.

A sample grouping or characteristic can be occupation-type. An occupation-type can be, e.g., healthcare, advertising, charity or voluntary work, education, administration, engineering, environment, financial management or accounting, agriculture, legal, hospitality, human resources, insurance, law enforcement, business, aviation, fishing, tourism, media, mining, performing arts, publishing or journalism, retailing, social care or guidance work, recreation, athletic, government, public service, science, or military, etc.

A sample grouping or characteristic can be annual income level. Annual income level can be, e.g., about $0-$20,000; $20,000-$40,000; $40,000-$60,000; $60,000-$75,000; $75,000-$100,000; $100,000-$150,000; $150,000-$200,000; $200,000-$500,000; $500,000-$1,000,000; $1,000,000-$10,000,000; $10,000,000-$100,000,000; or more than $100,000,000. Annual income level can be about, more than about, or less than about $2500, $5000, $7500, $10,000, $12,500, $15,000, $17,500, $20,000, $22,500, $25,000, $27,500, $30,000, $35,000, $40,000, $50,000, $60,000, $70,000, $80,000, $90,000, $100,000, $125,000, $150,000, $200,000, or $250,000.

A sample grouping or characteristic can include a factor related to diet. Factors related to diet can include, e.g., daily caloric intake, types of food consumed (e.g., proteins, carbohydrates, fruits, vegetables, meats, dairy products, sweets, desserts, saturated fat, unsaturated fat, cholesterol, etc.), schedule of meal consumption, etc.

A sample grouping or characteristic can be geographic location of a subject. A geographic location can be a street address, a city block, a neighborhood in a town or city, a town or city, a metropolitan area, a county, a state (e.g., any of the 50 states of the United States), a country, a continent, or a hemisphere. A test subject and a normal individual can live in the same geographic location.

A sample grouping or characteristic can be exposure to a disaster and/or environmental condition. A disaster or environmental condition can be, e.g., an earthquake, a hurricane, a blizzard, a flood, a tornado, a tsunami, a fire, air pollution, water pollution, a terrorist attack, a bioterrorist attack, radiation, nuclear attack, insect infestation, food contamination, asbestos, war, pandemic, lead poisoning, etc.

A sample from a test subject can be compared to a sample from one or more normal subjects that share one or more sample characteristics with the test subject.

### EXAMPLES

### Example 1

### Normal Cell Response to Erythropoietin (EPO) and Granulocyte Colony Stimulating Factor (G-CSF)

Normal cell signaling responses to EPO and G-CSF were characterized through comparison to signaling responses observed in samples from a subclass of patients with myelodysplatic syndrome (MDS) referred to herein as “low risk” patients. Fifteen samples of healthy BMMCs (from patients with no known diagnosis of disease) and 14 samples of BMMCs from patients who belonged to a subclass of patients with myelodysplastic syndrome were used to characterize normal cell responses to EPO and G-CSF. The 14 samples of low risk patients were obtained from MD Anderson Cancer Center in Texas. The low risk patients were diagnosed as per standard of care at MD Anderson Cancer Center. The 15 samples of healthy BMMCs were obtained through Williamson Medical Center and from a commercial source (AllCells, Emeryville, Calif.). The samples obtained through Williamson Medical Center were collected with informed consent from patients undergoing surgeries such as knee or hip replacements.

Each of the normal and the low risk samples were separated into aliquots. The aliquots were treated with a 3 IU/ml concentration of Erythropoietin, a 50 ng/ml concentration of G-CSF and both a 3 IU/ml concentration of Erythropoietin and a 50 ng/ml concentration of G-CSF. Activation levels of pStat1, pStat3 and pStat5 were measured using flow cytometry at 15 minutes after treatment with the modulators. In addition to the Stat proteins measured, several other elements were measured in order to separate the cells into discrete populations according to cell type. These markers included CD45, CD34, CD71 and CD235ab. CD45 was used to segregate lymphocytes, myeloid(p1) cells and nRBCs. The nRBCs were further segregated into 4 distinct cell populations based on expression of CD71 and CD235ab: m1, m2, m3 and m4.

Distinct signaling responses were observed in the different cell populations; different activation levels of pStat1, pStat3 and pStat5 were observed in EPO, G-CSF and EPO+G-CSF treated lymphocytes, nRBC1 cells, myeloid(p1) cells and stem cells (data not shown). Although this was true in both the healthy and the low risk patients, the different cell populations exhibited a much narrower range of induced activation levels in normal samples than in the low risk samples. The different cell populations also show a much narrower range of non-response to a modulator in normal cells. These observations accord with the common understanding that diseased cells exhibit a wider range of different signaling phenotypes than normal cells. Additionally, cell differentiation in disease may be inhibited or stunted, causing cells to exhibit characteristics such as signaling phenotypes that are different from other cells of the same type.

Different activation levels of EPO, G-CSF and EPO+G-CSF-induced pStat1, pStat3 and pStat5 were observed in cell populations at various stages of maturation into red blood cells. The healthy samples exhibit much less variance in the activation levels of pStat1, pStat3 and pStat5 than the low risk samples. Combining the modulators EPO and G-CSF does not alter this observation; the combined response to the modulators still exhibits less variance in the healthy cells. This result suggests that modulators may be combined prior to modulation without distorting the activation state data. These results demonstrate the utility of using the variance of the observed activation levels as a metric for diagnoses and/or prognoses.

### Example 2

### Normal Cell Response to PMA and IFNa

Normal cell signaling responses to PMA and IFNa were characterized in a set of 12 normal samples. Twelve of the normal samples were obtained from the National Institute of Health (NIH) and consisted of cryopreserved leukapheresis peripheral blood mononuclear cell (PBMC) samples. The normal samples had been previously categorized as high pStat5 responders and low pStat5 responders by the NIH based on flow-cytometry based analysis of IFNa-induced pStat5 in isolated T cells (measured at 15 minutes after modulation). The set of samples comprised 6 high responders and 6 low responders. The set of samples were homogeneous by gender and were blind associated with race, age, gender and pStat5 response. Additionally, two normal samples comprising cryopreserved PBMCs from healthy donors were processed at Nodality. In addition to the above described samples, a Jurkat cell line was used as a control.

Activation levels of different activatable elements were measured at different time intervals after stimulation with PMA and IFNa. In addition to the activatable elements, several cell type markers were used to segregate the single cell data for each sample into discrete cell populations. Two different phosphorylation sites on pStat1(Y701 and S727) and pStat3 (Y705 and 5727) were measured. Unless otherwise noted, pStat1 and pStat3 activation discussed herein refers to pStat1(Y701) and pStat3 (Y705),

Cell surface markers and other markers such as Live/dead amine Aqua stain were used to segregate the single cell data according to cell populations. First, live/dead amine Aqua stain was used to select for viable cells. CD14 was then used to segregate monocytes from lymphocytes. SSC-A, CD20 and CD3 were used to segregate T cells, B Cells and CD3-CD20-lymphocytes. CD4 was used to segregate T cells into CD4+ and CD4− T cells. The percentage recovery from the samples, a metric that compares the expected cell count to the actual cell count, was determined. The percentage viability of the cells in the samples was determined based on Aqua staining and the percentage of cells that express cleaved PARP (a marker for apoptosis). The percentage of cells that exhibit higher than average auto-fluorescence was compared to the percentage of cells that exhibit higher than average cleaved-PARP staining.

The different cell populations demonstrated different responses to stimulation with PMA. pS6 and pERK response after stimulation with PMA in T cells, B cells and monocytes, respectively was observed.

Response to IFNa was also unique to the cell population being observed. The fold change in pStat1, pStat3 and pStat5 between IFNa stimulated and unstimulated cells over time after stimulation was determined (data not shown). The fold change of the activatable elements was measured at 1, 15, 60, 120 and 240 minutes. In most of the cell populations and activatable elements observed, the average fold change peaks at 15 minutes post-stimulation. The fold change in pStat4, pStat6 and p-p38 between IFNa stimulated and unstimulated cells from the normal samples was determined (data not shown). In most of the cell types observed, the average fold change peaks at 60 minutes. In this experiment, pStat4 is only induced by IFNa in T cells (data not shown).

The IFNs-induced fold change in pStat1(S727) and pStat3(S727) in Monocytes, T cells and B cells from the normal samples was determined (data not shown). None of the different cell types demonstrated more than a minimal activation of pStat1(S727) and/or pStat3(S727). The IFNa-induced fold change in pStat1(S727) and pStat3(S727) in CD4+ and CD4− T cells was determined (data not shown). The magnitude of pStat5 fold change was much larger in CD4+ T cells (average fold change 7.2) than in CD4− T Cells (average fold change 3.2).

The IFNa-induced fold change in pStat4, pStat6 and p-p38 in CD4+ and CD4− T cells from the normal samples was determined (data not shown). The magnitude of pStat4 fold change was much larger in CD4− T cells (average fold change 1.8) than in CD4+ T Cells (average fold change 1.5).

The IFNa-induced activation of pStat1, pStat3, pStat5, pStat4, pStat6, p-p38, pStat3(S727) and pStat1(S727) in the Jurkat cells that were used as a control was determined (data not shown). These cells demonstrated minimal IFNa-induced activation of pStat4, pStat6, p-p38, pStat3(S727) and pStat1(S727). IFNa-induced activation of pStat1, pStat3 and pStat5 peaked at 15 minutes.

The IFNa-induced activation of pStat1, pStat3 and pStat5 in Jukat cells (control) and the T cells from the normal samples was determined (data not shown). The magnitude of the pStat3 fold change in the Jurkat cells (average fold change=4.3) was much larger than in the T cells (average fold change=3.2).

The relative frequencies of different cell sub-populations were determined (data not shown). IFNa-induced pStat1, pStat3, and pStat5 in monocytes, T cells and B cells were compared (data not shown). IFNa-induced pStat1, pStat3, and pStat5 in samples from a Jurkat cell line was determined (data not shown). The different colored bars represent different plates of samples from which the activation levels of IFNa-induced pStat1, pStat3, and pStat5 were measured. As shown in the bar graphs, there was good agreement between the activation levels in the two sets of control data.

The NIH Stat5 response classifications were determined (data not shown). These NIH response classifications were generated by stimulating isolated T cells from the samples with IFNa and measuring pStat5 response at 15 minutes. The agreement between the NIH response classifications and observed IFNa-induced pStat5 response was determined (data not shown). Of the 12 samples, the 3 samples with the highest IFNa-induced pStat5 response and the 3 samples with the weakest IFNa-induced pStat5 response corresponded with the NIH response classifications. However, the other samples did not agree. This difference may be explained by the fact that the T cells were isolated in the NIH experiment prior to characterizing pStat5 response, whereas in our analysis the T cells with modulated with pStat5 in a heterogeneous population of cells.

IFNa-induced pStat1, pStat3, and pStat5 in different cell populations as a function of the age of the person from whom the sample was derived was determined (data not shown). IFNa-induced pStat1, pStat3, and pStat5 in Monocytes as a function of age was determined (data not shown). IFNa-induced pStat1, pStat3, and pStat5 in T cells as a function of age was determined (data not shown). A strong T cell response was consistently observed in one of the samples (termed NIH10). IFNa-induced pStat1, pStat3, and pStat5 in B cells as a function of age was determined (data not shown). A strong B cell response was also observed in sample NIH10. These results illustrate the utility of sampling a large range of normal patients to develop a model of normal activation levels and using similarity rather than classification to characterize patients. A classification model based on the samples would be skewed by the high activation values observed in sample NIH10. However, a similarity based model would account for the fact that NIH10 is dissimilar in activation level to the other normal samples.

Correlations between age and IFNa-induced pStat4 and pStat6 activation levels were determined (data not shown). A positive correlation was observed between IFNa-induced pStat4 and age. A negative correlation was observed between IFNa-induced pStat6 and age. These results demonstrate that some induced activation levels for a test subject, e.g., an undiagnosed individual, can be normalized according to the age of the individual prior to determining the similarity to normal samples.

IFNa-induced pStat1, pStat3 and pStat5 activation levels in monocytes, B cells and T cells derived from normal samples from European Americans (ea) and African Americans (aa) were determined (data not shown). No differences associated with race were observed.

The correlation between observed activation levels in the different cell populations in the normal samples were determined (data not shown). The Pearson correlation coefficient was calculated using difference metric (i.e., the difference between the Mean Fluorescence values in stimulated and unstimulated samples) to represent the activation levels. Positive correlations greater than or equal to 0.5 and negative correlations less than or equal to −0.5 were determined. Generally, very high correlation was observed between the pStat1, pStat3 and pStat5 in the B cells and the T cells. The correlations between nodes in different cell populations were illustrated using a circular plot, where nodes with a positive correlation (>0.5) are connected by a red line and nodes with a negative correlation (<=−0.5) are connected by a green line.

The similarity in activation profiles between the normal samples were determined with heat maps (data not shown). The activation levels of the nodes in different cell populations were normalized by the maximum and minimum activation level (represented by the difference metric) for each node such that all nodes range from 0 to 1. Although little variance was exhibited in the samples, this normalization method magnifies the existing variance such that the samples may be analyzed to determine whether there are distinct subgroups of normal samples. These results suggest that it may be helpful to build multiple models for normal samples according to the different subgroups of response observed.

### Example 3

### Normal Cell Response to Varying Concentrations of GM-CSF, IL-27, IFNa and IL-6 in Whole Blood

Kinetic response to varying concentrations of modulators was investigated in normal whole blood samples (i.e., samples from persons who have no diagnosis of disease). 11 normal samples were donated with informed consent by Nodality Inc. employees and processed at Nodality Inc. in South San Francisco, Calif. The samples were treated with 4 different modulators (GM-CSF, IL-27, IFNa and IL-6) at 4 different concentrations of the modulator and activation levels of pStat1, pStat3 and pStat5 were measured at different time points. Activation levels were measured at 3, 5, 10, 15, 30 and 45 minutes using flow cytometry-based single cell network profiling. The concentrations of the stimulators are tabulated below in Table 2:

TABLE 2
Stimulator Concentrations

| | low | | med | | hi | |
|---|---|---|---|---|---|---|
| GM-CSF | 0.1 | ng/ml | 1 | ng/ml | 10 | ng/ml |
| IL-27 | 1 | ng/ml | 10 | ng/ml | 100 | ng/ml |
| IFNa | 1000 | IU | 4000 | IU | 100000 | IU |
| IL-6 | 1 | ng/ml | 10 | ng/ml | 100 | ng/ml |

The activation levels of pStat1, pStat3 and pStat5 were measured in discrete cell populations as defined by cell surface receptor expression. Gating was used to segregate the cells into discrete cell populations. In the gating analysis, SSC-A and FSC-A were first used to segregate lymphocytes from non-lymphocytes. CD14 and CD4 were then used to segregate the non-lymphocytes into populations of neutrophils and CD14+ cells (monocytes). CD3 and CD20 were then used to segregate the lymphocytes into populations of CD20+ (B Cells), CD3+ (T Cells) and CD20-CD3− cells. CD-4 was used to segregate the CD3+ T cells into populations of CD3+ CD4− and CD3+ CD4+ T cells.

The kinetic responses of different cell populations in the normal samples were determined (data not shown). The activation levels observed in all of the donors over the time intervals at which they were measured were determined (data not shown). The activatable elements may have varying responses based on the concentration of the modulator. The activation levels for the different samples showed little variation across donors for the same concentration of IL-6. This suggests tight regulation of phosphorylation in normal cells.

The kinetic responses of different cell populations in the normal samples were determined (data not shown). The line graphs contained in plot the activation levels observed in all of the donors over the time intervals at which they were measured. The different concentrations of IL-6 tabulated above are represented by different colored lines. Generally, the normal samples demonstrated similar activation profiles over time according to the concentration of sample given. Different concentrations of the modulator IL-6 yielded dramatically different activation profiles for some of the Stat phosphoproteins measured. For example, IL-6-induced pStat3 response varied at early time points (5-15 minutes) for the different concentrations of IL-6 but became more uniform at later time points. This uniformity of response supports the idea that normal cells exhibit a narrow range of activation. As the different cell populations exhibited very different signaling profiles, these results also demonstrate the utility of segregating single-cell data into discrete cell populations prior to analysis.

Different cell populations demonstrated unique responses to modulation. The neutrophils exhibited very low IL-6 induced activation as compared to the CD4+ T cells and monocytes. Between the CD4+ T cells and monocytes, several differences in activation profiles were observed. Monocytes showed a peak activation of IL-6-induced pStat1 activity at a different time point than the CD4+ T cells. Although both the monocytes and the CD4+ T cells demonstrated a drop-off in pStat3 activity after 15 minutes, the drop-off (post-peak or “resolution phase” activity) was much more dramatic in the monocytes (data not shown). This observation confirms the utility of using additional metrics which describe the dynamic response such as ‘slope’ and liner equations to represent dynamic response to induced activation.

The different activation profiles for IFNa and IL-6-induced pStat1, pStat3 and pStat5 in T cells were compared (data not shown). IFNa can activate all three Stats with activation profiles that are correlated over time. This result implies that IFNa induced Stat profiles that are not positively correlated may indicate dysregulation of Stat signaling or disease. In contrast, IL-6 induced Stat signaling did not show positively correlated activation profiles over time.

Cell population dependent differences in IFNa induced and GM-CSF-induced Stat profiles were investigated (data not shown). IFNa-2b-induced pStat1, pStat3 and pStat5 showed a range of activation profiles in monocytes; there was little to no activation of IFNa-2b-induced pStat1 and pStat5 in neutrophils (data not shown). The two cell populations showed more similar response to GM-CSF modulation. However, the activation profiles indicate that neutrophils have prolonged activation phase of pStat5 responsive to G-CSF induction, whereas monocytes demonstrate a resolution phase after 15 minutes.

GM-CSF, IFNa-2b, IL-6 and IL-27 induced pStat1, pStat3 and pStat5 in neutrophils, monocytes, CD4+ T cells, CD4− T cells, and Non B/T Cell lymphocytes (NK) were investigated. These results demonstrate the utility of capturing different concentrations of different modulators at different time points: many of cell populations that are uniquely responsive to different modulator and activation levels show little variance associated in some cell types/concentrations of modulators. Both of these properties allow for the characterization and modeling of normal cell activity. Unique response (including non-response) to modulators based on cell type allows for the identification of aberrant differentiation and signaling dysregulation. Invariant response similarly allows for the identification of outlier activation levels that may be associated with disease.

IL-6 induced activation of pStat4 in CD3+ CD4+ T cells was investigated over time. Staining controls included bulk IFN-alpha dose response from one donor. While different activation levels were associated with the different concentrations of IL-5 at earlier time points, a convergence of the activation levels at 15 minutes time was observed. Although the different concentrations are still distinguishable at 15, 30 and 45 minutes, the ranges observed with the different concentrations demonstrate far less variance. These data demonstrate activation ranges that may serve as unique, low variance indicators of disease and/or dysregulation independent of the concentration of modulator used to induce the activation levels.

### Example 4

### Functional Pathway Analysis of the Healthy Immune System

A greater understanding of the function of the human immune system at the single cell level in healthy individuals can play a role in discerning aberrant cellular behavior that can occur in settings such as autoimmunity, immunosenescence, and cancer. To achieve this goal, a systems-level approach capable of capturing responses of interdependent immune cell types to external stimuli can be used. In this study, an extensive characterization of signaling responses in multiple immune cell subpopulations within PBMCs from a cohort of 60 healthy donors was performed using single cell network profiling (SCNP). SCNP can be a multiparametric flow-cytometry based approach that can enable the simultaneous measurement of basal and evoked signaling in multiple cell subsets within heterogeneous populations. In addition to establishing the inter-individual degree of variation within immune signaling responses, the possible association of any observed variation with demographic variables including age and race was investigated. Using half of the donors as a training set, multiple age- and race-associated variations in signaling responses in discrete cell subsets were identified, and several were subsequently confirmed in the remaining samples (test set). Such associations can provide insight into age-related immune alterations associated with high infection rates and diminished protection following vaccination and into the basis for ethnic differences in autoimmune disease incidence and treatment response. SCNP allowed for the generation of a functional map of healthy immune cell network responses that can provide clinically relevant information regarding both the mechanisms underlying immune pathological conditions and the selection and effect of therapeutics.

A systems-level approach can be used to provide a comprehensive understanding of how the function of the human immune system arises from the interactions among numerous inter-connected components, pathways, and cell types. Reductionist approaches that analyze individual components within the immune system have dominated in the past several decades primarily due to technological limitations. The recent development of high-throughput technologies is beginning to change the landscape of immunological studies and researchers are ushering in the new field of systems immunology (1). Here, a novel technology is described that can have an enormous impact on this burgeoning field because it can allow for simultaneous functional measurements from multiple cell subpopulations without the need for prior cell separation. This capability can enable a more integrated description of immune function than traditional studies which often focus on the behavior of specific cell types that have been physically isolated from heterogeneous tissues such as peripheral blood, spleen, or lymph nodes. This technology was applied to the characterization of immune cell signaling in healthy individuals to establish a reference functional map in the context of an immune cell signaling network, which can be used to elucidate aberrant network-level behaviors underlying the pathogenesis of immune-based diseases.

SCNP can be a multiparametric flow-cytometry based analysis that can simultaneously measure, at the single cell level, both extracellular surface markers and changes in intracellular signaling proteins in response to extracellular modulators. Measuring changes in signaling proteins following the application of an external stimulus informs on the functional capacity of the signaling network which cannot be assessed by the measurement of basal signaling alone (2). In addition, the simultaneous analysis of multiple pathways in multiple cell subsets can provide insight into the connectivity of both cell signaling networks and immune cell subtypes (3). SCNP technology can be used to investigate signaling activity within the many interdependent cell types that make up the immune system because it can allow for the simultaneous interrogation of modulated signaling network responses in multiple cell subtypes within heterogeneous populations, such as PBMCs, without the additional cellular manipulation that can be used for the isolation of specific cell types.

Summarized below are the results of an extensive characterization of immune cell signaling responses utilizing SCNP technology to quantify phospho-protein levels (pStat1, pStat3, pStat5, pStat6, pAkt, pS6, pNFκB, and pErk) within pathways downstream of a broad panel of immunomodulators (including IFNα, IFNγ, IL2, IL4, IL6, IL10, IL27, α-IgD, LPS, R848, PMA, and CD40L) in seven distinct immune cell subpopulations within PBMC samples from 60 healthy adults. This systems-level approach enabled the generation of a functional map of immune cell network responses in healthy individuals which serves as a reference for understanding signaling variations that occur in pathological conditions such as autoimmunity and to inform clinical decision-making in vaccination and other immunotherapeutic settings. In addition, inter-subject variation in immune signaling responses associated with demographic characteristics of the healthy donors such as age or race was identified.

Materials and Methods

PBMC Samples

Cryopreserved PBMC samples taken from 60 healthy donors within the Department of Transfusion Medicine, Clinical Center, National Institutes of Health with Institutional Review Board approval were used in this study (Table 3). Blood donations from healthy donors, donated for research purposes with informed consent, were collected and processed as described previously (4).

TABLE 3
Summary of donor numbers, age, race, and gender in the
master, training, and test sample sets

| | Master | Training | Test |
|---|---|---|---|
| Number of | 60 | 30 | 30 |
| Donors | | | |
| Mean Age | 48.9 (19-73) yrs | 47.9 (22-73) yrs | 49.8 |
| (Range) | | | (19-73) yrs |
| Gender | 12 Female | 5 Female | 7 Female |
| | 48 Male | 25 Male | 23 Male |
| Race | 25 African American | 10 African American | 15 African |
| | 34 European American | 19 European American | American |
| | 1 Hispanic | 1 Hispanic | 15 European |
| | | | American |
| | | | 0 Hispanic |

SCNP Assay

Cryopreserved PBMC samples were thawed at 37° C. and resuspended in RPMI 1% FBS before staining with amine aqua viability dye (Invitrogen, Carlsbad, Calif.). Cells were resuspended in RPMI 10% FBS, aliquoted to 100,000 cells per well of 96-well plates, and rested for 2 h at 37° C. prior to 15 min 37° C. incubation with the following modulators: 1000 IU/ml IFNα (PBL, Piscataway, N.J.); 250 ng/ml IFNγ, 50 ng/ml IL4, 50 ng/ml IL10, α-IgD 5 μg/ml (BD, San Jose, Calif.); 50 ng/ml IL2, 50 ng/ml IL6, 50 ng/ml IL27, CD40L 0.5 μg/ml (R&D, Minneapolis, Minn.); R848 5 μg/ml (Invivogen, San Diego, Calif.); LPS 1 μg/ml, PMA 40 nM (Sigma Aldrich, St. Louis, Mo.). After exposure to modulators, cells were fixed with paraformaldehyde and permeabilized with 100% ice-cold methanol as previously described (5). Methanol permeabilized cells were washed with FACS buffer (PBS, 0.5% BSA, 0.05% NaN₃), pelleted, and stained with fluorochrome-conjugated Abs. Abs used include α-CD3 (clone UCHT1), α-CD4 (clone RPA-T4), α-CD45RA (clone HI100), α-CD20 (clone H1), α-pNFκB (clone K10-895.12.50), α-cPARP (clone F21-852), α-pStat1 (clone 4a), α-pStat3 (clone 4/p-Stat3), α-pStat5 (clone 47), α-pStat6 (clone 18/p-Stat6), α-pErk (clone 20A) [BD, San Jose Calif.]; α-pAtk (clone D9E), α-pS6 (clone 2F9) [CST, Danvers, Mass.]; and α-CD14 (clone RMO52) [Beckman Coulter, Brea, Calif.].

Flow Cytometry Data Acquisition and Analysis

Flow cytometry data was acquired using FACS DIVA software (BD, San Jose, Calif.) on two LSRII Flow Cytometers (BD, San Jose, Calif.). All flow cytometry data were analyzed with WinList (Verity House Software, Topsham, Me.). For all analyses, dead cells and debris were excluded by forward scatter (FSC), side scatter (SSC), and amine aqua viability dye. PBMC subpopulations were delineated according to an immunophenotypic gating scheme (not shown).

SCNP Terminology and Metrics

The term “signaling node” can refer to a specific protein readout in the presence or absence of a specific modulator. For example, a response to IFNα stimulation can be measured using pStat1 as a readout. This signaling node can be designated “IFNα→pStat1”. Each signaling node can be measured in each cell subpopulation. The cell subpopulation can be noted following the node, e.g., “IFNα→Stat1 B cells”. Two different metrics are utilized in this study to measure the levels of intracellular signaling proteins in either the unmodulated state or in response to modulation. The “Basal” metric is used to measure basal levels of signaling in the resting, unmodulated state. The “Fold” metric is applied to measure the level of a signaling molecule after modulation compared to its level in the basal state. The Equivalent Number of Reference Fluorophores (ERFs), fluorescence measurements calibrated by rainbow calibration particles on each 96-well plate, serve as a basis for all metric calculations (6, 7).

The “Basal” and “Fold” metrics were calculated as follows:

log₂[ERF(Unmodulated)/ERF(Autofluorescence)] Basal:

(log₂[ERF(Modulated)/ERF(Unmodulated)]+Ph−1)/Ph Fold:

Where Ph is the percentage of healthy [cleaved PARP (poly ADP-ribose polymerase) negative] cells

Statistical Analysis

The high dimensionality of the SCNP data for individual nodes (i.e., combination of cell populations, modulators, and protein readouts) greatly increases the probability of finding chance associations in the data (i.e., false discovery). To address this issue, a multi-step analysis strategy designed to reduce the chance of false discoveries, by accounting for multiple testing and therefore reducing the chance of a Type 1 Error (incorrectly rejecting the null hypothesis) was followed. First, the data was split into training (30 samples) and test sets (30 samples) stratified randomly on race and age (Table 3). Multivariate linear regression was then used to find associations between individual immune signaling nodes and age and/or race in the training set. Associations with immune signaling were found by controlling for age and race. The exact form of the linear model used to test for significant associations between age, race and node signaling in the training data set was:

SignalingNode|Population=α₁+Age*β₁+Race*β₂

Where Race was coded as (1=African American, 0=European American). Linear models were built for each signaling node in each of the following cell subpopulations: monocytes, B cells, naïve helper T cells, naïve cytotoxic T cells, memory helper T cells, and memory cytotoxic T cells. In the training data set, signaling nodes were considered to have a significant association with age for models in which β₁ has a significant p-value (<0.05) and a significant association for race for models in which β₂ has a significant p-value (<0.05). Discovering groups of signaling nodes rather than individual nodes can guard against finding chance associations. To create groupings of nodes, a principal component analysis (PCA, (8)) was performed both on the set of immune signaling nodes found to be significantly associated with age and also with the set of immune signaling nodes found to be significantly associated with race from the linear models in the training data. The PCA analysis accounted for correlation among signaling nodes, which can carry redundant information, by creating linear combinations of signaling nodes associated with age and/or race. In addition, to confirm the age and race associations in the test set a Gatekeeper strategy was used to control the Type 1 Error rate (9). In this strategy, each hypothesis to be validated in the test set can be pre-specified and sequentially ordered and subsequently tested in that order. A hypothesis can be considered validated if it is significant in the test set and all other hypotheses tested prior to it are significant. For this study, models using the first principal component from the age PCA and the first principal component from the race PCA were tested in the test set. The principal component models for age and race which were locked (i.e., the model coefficients and PCA loadings matrices were locked) in the training set before being tested on the test set (in order) were of the form:

Race=α₁+NodePC₁*β₁+Age*β₂

NodePC₁=α₁+Age*β₁+Race*β₂

Only the first principal components were tested since both first principal components for both the age and race PCA both accounted for approximately 50% of the variance in training data. Only after the confirmation of the principal components in the test set were the contributions of the individual signaling nodes to the principal components for age and race associations examined, to understand the biology associated with age and/or race.

### Correlations Between Signaling Nodes

R software (version 2.12.1) was used to compute Pearson correlation coefficients between all pairs of signaling nodes within and between each of the seven distinct cell subpopulations. Heatmaps were generated in Excel 2007 (Microsoft, Redmond, Wash.).

Results

Cell-type-specific Patterns of Immune Signaling Responses in PBMCs from Healthy Donors

Thirty eight signaling nodes, or specific protein readouts in the presence or absence of a specific modulator (Table 4), were measured in 12 cell populations defined by their surface phenotypes including 7 distinct immune cell subpopulations (monocytes, B cells, CD3-CD20-lymphocytes (NK cell-enriched subpopulation), naïve helper T cells, memory helper T cells, naïve cytotoxic T cells, and memory cytotoxic T cells, (data not shown)) within unsorted PBMC samples from 60 healthy donors (Table 3) using two different metrics [Basal and Fold (Materials and Methods)].

TABLE 4
Thirty-eight signaling nodes measured in the study. All signaling
nodes were measured in each immune cell subpopulation.

| | Signaling Node |
|---|---|
| 1 | IFNα → pStat1 |
| 2 | IFNα → pStat3 |
| 3 | IFNα → pStat5 |
| 4 | IFNα → pStat6 |
| 5 | IFNγ → pStat1 |
| 6 | IFNγ → pStat3 |
| 7 | IFNγ → pStat5 |
| 8 | IFNγ → pStat6 |
| 9 | IL2 → pStat5 |
| 10 | IL2 → pStat6 |
| 11 | IL4 → pStat5 |
| 12 | IL4 → pStat6 |
| 13 | IL6 → pStat1 |
| 14 | IL6 → pStat3 |
| 15 | IL10 → pStat1 |
| 16 | IL10 → pStat3 |
| 17 | IL27 → pStat1 |
| 18 | IL27 → pStat3 |
| 19 | IL27 → pStat5 |
| 20 | IL27 → pStat6 |
| 21 | α-IgD/LPS → pS6 |
| 22 | α-IgD/LPS → pAkt |
| 23 | R848 → pErk |
| 24 | R848 → pNFκB |
| 25 | CD40L → pErk |
| 26 | CD40L → pNFκB |
| 27 | PMA → pS6 |
| 28 | PMA → pErk |
| 29 | Unmodulated → pStat1 |
| 30 | Unmodulated → pStat3 |
| 31 | Unmodulated → pStat5 |
| 32 | Unmodulated → pStat6 |
| 33 | Unmodulated → pS6 |
| 34 | Unmodulated → pAkt |
| 35 | Unmodulated → pErk |
| 36 | Unmodulated → pNFκB |
| 37 | Unmodulated (DMSO) → pS6 |
| 38 | Unmodulated (DMSO) → pErk |

When gating on the viable cells (defined by scatter properties and amine aqua as described in Materials and Methods) only 15 of the 28 modulated signaling nodes showed a signaling response above the threshold level of Fold >0.25 representing an approximately 1.2 fold change in modulated levels relative to basal (see Materials and Methods), and a level of signaling that is very reproducible (data not shown). In contrast, when gating separately in the same samples on the 7 distinct immune cell subpopulations, 23 of these nodes showed induced signaling in at least one of the 7 subpopulations (data not shown), exemplifying the utility of SCNP in the identification of heterogeneous functionality in complex tissues and rare cell populations.

Other examples support this conclusion (data not shown). The TLR ligand R848 (Resiquimod) can be an immunomodulator that can portray cell-type specificity, and consistent with this induced pErk and pNfκB only in B cells and monocytes, immune cell subpopulations known to express the receptors (TLR7/8) for this ligand. In contrast to R848, IFNα can be a globally active immunomodulator due to the ubiquitous expression of the IFNα receptor on immune cells. As expected, at least one pStat protein was activated in response to IFNα in all of the immune cell subpopulations (data not shown) and this global responsiveness was reflected in the data from the Viable Cell population. Due to the generally reduced signaling responses from the more heterogeneous parental populations, in the sections below, data is reported primarily for the 7 distinct immune cell subpopulations.

Since the SCNP assay allows for an actual quantification of signaling responses, by measuring the degree of pathway activity for each node in each cell subpopulation, differential levels of activation in the different immune cell subtypes was observed. For example, as expected, modulation of PBMCs with IFNγ produced the highest level of pStat1 in monocytes, lower levels in B cells, and a much weaker pStat1 response in T cells (with differential levels of activation among the latter, i.e., naïve T cell subsets showing a higher level of response than their memory counterparts (data not shown). In contrast to IFNγ treatment, IL2 modulation of PBMCs led to pStat5 activation primarily in CD3-CD20-lymphocytes and T cells, again with differential activation levels seen among the T cell subsets and no effects on monocytes and B cells (data not shown).

Variation in Immune Signaling Responses in PBMCs from Healthy Individuals

For each of the 38 signaling nodes tested in the assay (listed in Table 4), the range of signaling responses in each immune cell subset across the 60 samples was quantified (data not shown). A comparison of the data obtained from the analysis of the training set and the test set revealed that, as expected, the distributions in the training and test set did not differ significantly for a majority of the signaling responses (p>0.05 for 98.9% of the 38 signaling nodes measured within each of the 7 distinct cell subsets). Although there was a narrow range of responses for the majority of the signaling nodes measured within the 7 distinct cell subsets, considerable inter-donor variation was observed for a subset of the modulated nodes (data not shown).

Immune Cell Signaling Network Map in PBMCs from Healthy Individuals

A functional map of the healthy immune cell signaling network was generated by calculating the Pearson correlation coefficients between pairs of nodes within and between each of the 7 distinct immune cell subpopulations. Overall, visualization of the healthy immune cell signaling network map revealed a high frequency of positively correlated signaling responses (data not shown). Cytokine-induced signaling responses within each subpopulation were highly positively correlated, with a notable exception occurring for the naïve cytotoxic T cell subset for which IL10 and IL2 signaling responses were uncorrelated or weakly inversely correlated with responses to other cytokines (data not shown). Positive correlations among cytokine signaling responses were also present across different cell subpopulations with the strongest inter-subpopulation correlations generally occurring between pairs of nodes within the different T cell subsets. Intra-subpopulation correlations among cytokine-induced signaling responses and among PMA-induced signaling responses were weakest within the B cell subset, although strong positive correlations were present for signaling responses downstream of CD40L and between responses downstream of IgD crosslinking in this subpopulation.

Age and/or Race as Variables Associated with Immune Signaling Responses

Both age and race are known to be relevant to clinical outcomes in immune based disorders (10-12). Demographic heterogeneity of the 60 donor cohort (Table 3) allowed us to assess the association between immune signaling responses and age and/or race. Given the large dimensionality of the SCNP data for individual nodes (i.e., combination of cell populations, modulators, and protein readouts) the possibility of chance association (i.e., false discovery) is high. To address this issue, we followed a multi-step analysis strategy. First, the data was split into training (30 samples) and test sets (30 samples) randomly stratified on race and age. Multivariate linear regression was then used to find associations between individual immune signaling nodes and age and/or race in the training set. Because discovering groups of signaling nodes can guard against chance associations, a principal component analysis (PCA) was performed both on the set of immune signaling nodes associated with age and the set of signaling nodes associated with race. The PCA analysis accounted for the previously observed correlation among signaling nodes by combining the correlated signaling nodes associated with age or race in the training set. For confirmation of associations in the test set, a Gatekeeper strategy was used. The first principal component for both the age and race PCAs in the training set were locked and applied to the test set in a pre-specified order and significance level (p<0.05). Only after the confirmation of the principal components in the test set were the contributions of the individual signaling nodes to the principal components for age and race associations examined, to understand the biology associated with age and/or race.

The PCA for age-associated immune signaling was performed on 19 signaling responses found to be associated with age, controlled for race, in the training set (p<0.05, Table 5).

TABLE 5
Summary of age-associated signaling nodes identified in the training set. All age-associated
responses identified in the training set are shown, and nodes which were confirmed in the test
set are highlighted in gray. A negative slope indicates a negative correlation with age.

| | Training | | | Test | | |
|---|---|---|---|---|---|---|
| | | Age | Age p- | | Age | Age p- |
| Node\|Population | R² | slope | Value | R² | slope | Value |
| IFNα → pStat1\|Naïve cytotoxic T cells | 0.434 | −0.014 | 0.000 | 0.129 | −0.012 | 0.069 |
| IFNα → pStat3\|Naïve cytotoxic T cells | 0.249 | −0.006 | 0.013 | 0.043 | −0.003 | 0.399 |
| IFNα → pStat5\|Naïve cytotoxic T cells | 0.325 | −0.013 | 0.002 | 0.206 | −0.016 | 0.017 |
| IFNα → pStat6\|Memory helper T cells | 0.644 | −0.002 | 0.031 | 0.003 | 0.000 | 0.875 |
| IFNγ → pStat1\|Naive cytotoxic T cells | 0.422 | −0.007 | 0.000 | 0.131 | −0.005 | 0.074 |
| IL10 → pStat3\|Naive cytotoxic T cells | 0.201 | 0.010 | 0.022 | 0.059 | 0.005 | 0.368 |
| IL2 → pStat5\|Naive helper T cells | 0.539 | 0.027 | 0.000 | 0.201 | 0.023 | 0.022 |
| IL2 → pStat6\|Naive helper T cells | 0.291 | −0.007 | 0.011 | 0.122 | 0.004 | 0.176 |
| IL27 → pStat1\|Naive cytotoxic T cells | 0.310 | −0.026 | 0.010 | 0.076 | −0.017 | 0.168 |
| IL27 → pStat5\|Naive cytotoxic T cells | 0.234 | −0.010 | 0.011 | 0.222 | −0.009 | 0.016 |
| IL27 → pStat6\|Naive cytotoxic T cells | 0.278 | −0.003 | 0.049 | 0.009 | −0.001 | 0.678 |
| IL4 → pStat6\|Naive cytotoxic T cells | 0.187 | −0.012 | 0.026 | 0.234 | −0.013 | 0.020 |
| IL6 → pStat1\|Naive cytotoxic T cells | 0.342 | −0.009 | 0.002 | 0.129 | −0.008 | 0.074 |
| IL6 → pStat3\|Naive cytotoxic T cells | 0.340 | −0.016 | 0.003 | 0.082 | −0.014 | 0.148 |
| PMA → pErk\|B cells | 0.201 | 0.009 | 0.040 | 0.005 | −0.001 | 0.816 |
| PMA → pErk\|Naïve helper T cells | 0.331 | 0.012 | 0.026 | 0.005 | −0.001 | 0.816 |
| Unmodulated → pS6\|Memory cytotoxic T cells | 0.199 | −0.002 | 0.020 | 0.028 | −0.001 | 0.519 |
| Unmodulated (DMSO) → pS6\|Memory cytotoxic T cells | 0.167 | −0.002 | 0.036 | 0.064 | −0.001 | 0.208 |
| Unmodulated → pStat1\|Memory cytotoxic T cells | 0.201 | 0.002 | 0.038 | 0.114 | 0.001 | 0.245 |

The first principal component for age accounted for 45% of the variance. Examination of the 19 individual signaling nodes revealed that one of these responses (PMA→pErk|B cells) was within the B cell subpopulation, while all of the remaining responses were within T cell subsets with the highest number occurring within the naïve cytotoxic T cell subset. Only 3 unmodulated nodes (Unmodulated→pS6|Memory cytotoxic T cells, Unmodulated (DMSO)→pS6|Memory cytotoxic T cells, and Unmodulated→pStat1|Memory cytotoxic T cells, Table 5) were found to be associated with age in the training set.

The PCA for race-associated immune signaling included 18 signaling responses found to be associated with race, controlled for age, in the training set (p<0.05, Table 6).

TABLE 6
Summary of race-associated signaling nodes identified in the training set. All of the race-associated
responses identified in the training set are shown, and nodes which were confirmed in the test set
are highlighted in gray. A positive slope indicates nodes that were more responsive in AAs than in EAs.

| | Training | | | Test | | |
|---|---|---|---|---|---|---|
| | | Race | Race p- | | Race | Race p- |
| Node\|Population | R² | slope | Value | R² | slope | Value |
| IFNα → pStat3\|Memory cytotoxic T cells | 0.224 | 0.140 | 0.016 | 0.133 | −0.054 | 0.314 |
| IFNα → pStat3\|Memory helper T cells | 0.198 | 0.110 | 0.030 | 0.111 | −0.018 | 0.751 |
| IFNα → pStat5\|Monocytes | 0.343 | 0.100 | 0.025 | 0.015 | −0.038 | 0.534 |
| IFNα → pStat5\|Naïve helper T cells | 0.293 | 0.170 | 0.047 | 0.182 | −0.117 | 0.280 |
| IFNγ → pStat1\|Memory helper T cells | 0.234 | 0.060 | 0.048 | 0.032 | −0.015 | 0.699 |
| α-IgD + LPS → pAkt\|B cells | 0.386 | −0.390 | 0.001 | 0.265 | −0.347 | 0.006 |
| α-IgD + LPS → pS6\|B cells | 0.277 | −0.680 | 0.008 | 0.228 | −0.617 | 0.018 |
| IL10 → pStat1\|Memory helper T cells | 0.187 | 0.050 | 0.024 | 0.097 | −0.038 | 0.126 |
| IL10 → pStat3\|Memory cytotoxic T cells | 0.244 | 0.280 | 0.018 | 0.034 | −0.084 | 0.439 |
| IL10 → pStat3\|Memory helper T cells | 0.174 | 0.200 | 0.047 | 0.003 | −0.026 | 0.816 |
| IL27 → pStat1\|Memory cytotoxic T cells | 0.288 | 0.350 | 0.008 | 0.028 | −0.004 | 0.975 |
| IL27 → pStat3\|Memory cytotoxic T cells | 0.357 | 0.240 | 0.003 | 0.008 | −0.026 | 0.671 |
| IL6 → pStat1\|Memory cytotoxic T cells | 0.335 | 0.080 | 0.002 | 0.044 | −0.030 | 0.278 |
| IL6 → pStat3\|Memory cytotoxic T cells | 0.297 | 0.290 | 0.006 | 0.031 | −0.051 | 0.406 |
| IL6 → pStat3\|Memory helper T cells | 0.182 | 0.280 | 0.031 | 0.014 | −0.057 | 0.717 |
| R848 → pNFκB\|B cells | 0.279 | −0.090 | 0.021 | 0.040 | −0.027 | 0.579 |
| R848 → pNFκB\|Memory helper T cells | 0.258 | 0.030 | 0.016 | 0.121 | 0.008 | 0.603 |
| Unmodulated → pStat5\|Memory cytotoxic T cells | 0.568 | 0.039 | 0.043 | 0.017 | −0.002 | 0.881 |

The first principal component for race accounted for 54% of the variance. The 18 race-associated signaling responses consisted of a slightly more diverse set of cell subpopulations than the age-associated responses and included responses to several cytokines, the TLR ligand R848, and IgD crosslinking. Only one unmodulated node (Unmodulated→Stat5|Memory cytotoxic T cells) was associated with race in the training set.

The first principal component for age (locked from the training set) was significant in the test set (p<0.05), confirming that age can explain some of the observed inter-donor variation in immune signaling responses. After confirmation, this first principal component was dissected by inspecting the loadings matrix and whether or not the node was significant in both the test and training set, to further examine the underlying biology. Four individual signaling responses (IFNα→pStat5|Naïve cytotoxic T cells, IL27→pStat5|Naive cytotoxic T cells, IL4→pStat6|Naive cytotoxic T cells, IL2→pStat5|Naive helper T cells, Table 5) were found to have high loadings and were significantly associated with signaling in the test set as well. Of note, none of the unmodulated nodes with age-associations in the training set were individually significant in the test set. Exemplifying the SCNP assay advantage of subpopulation analysis, we confirmed that the IL4→pStat6 signaling node demonstrated a statistically significant decrease with age specifically within naïve cytotoxic T cells (data not shown; Table 5). A trend of decreasing signaling response with age was seen one level up the population hierarchy in the overall cytotoxic T cells, but this association was dampened by the memory cytotoxic T cells whose IL4→pStat6 signaling response showed no association with age and thus did not reach statistical significance in the overall cytotoxic T cell subset (data not shown). All 3 signaling nodes within the naïve cytotoxic T cell compartment (IFNα→pStat5, IL27→pStat5, and IL4→pStat6) were positively correlated with each other and all showed decreased responsiveness with age (Table 5, data not shown), while IL2→pStat5 activation within naive helper T cells increased with age and was uncorrelated with the three naïve cytotoxic T cell signaling nodes (Table 5, data not shown).

The race model, based on the first principal component for race (locked from the training set), was also significant in the test set (p<0.05), confirming that race is associated with differences in immune signaling responses (data not shown). After confirmation, this first principal component was also dissected to further examine the underlying biology. Two individual race-associated responses had high loadings and were significant in both the test and training sets. Both of these were within the B cell population (α-IgD/LPS→pAkt and α-IgD/LPS→pS6 nodes, data not shown, Table 6) and both showed greater levels of responsiveness in the European American (EA) donors than in the African American (AA) donors (data not shown), and they were highly correlated (r=0.81).

Defining the range of immune signaling activity in multiple immune cell subsets and establishing an overall map of the immune cell signaling network in healthy individuals can be used as a first step in providing a baseline for the characterization of aberrant signaling responses and changes in the immune signaling network architecture that occur in diseases such as cancer and autoimmune disorders. Because the immune system consists of multiple interdependent cell types whose behavior is mediated by complex intra- and inter-cellular regulatory networks, a comprehensive description of healthy immune function can use a systems-level approach capable of integrating information from multiple cell types, signaling pathways, and networks. In this Example, SCNP was used to perform a broad functional characterization of the healthy immune cell signaling network. As expected, many of the immunomodulators included in this study evoked cell-type specific responses (data not shown), highlighting the complexity of the regulation of biological function during immune responses. For a subset of the modulators and specific cell types investigated in this study, differential receptor expression and/or differential activation patterns have been previously reported. In instances where such data is available, the cell-type specific signaling responses described here are generally consistent with those reports (13-15).

To gain insight into the connectivity of the immune cell signaling network, node-to-node correlations within and between each of the distinct immune cell subpopulations were mapped. A high-level analysis of this map revealed an abundance of positively correlated nodes, with a higher frequency of positive correlations for node-to-node pairs within the same immune cell subset than for pairs of nodes spanning different cell types (data not shown). Very few nodes were inversely correlated with the most notable exceptions occurring for IL10- and IL2-induced responses which showed weak inverse correlations with other cytokine-induced signaling responses specifically within the naïve cytotoxic T cell subset. This map can be compared with those generated using samples from patients with immune-based disorders to identify changes in the network architecture that occur under pathological conditions, and can be applied to the analysis of samples obtained longitudinally from treated patients to monitor individual responses to therapeutics.

Aging is often accompanied by a deterioration of the immune system, resulting in a higher susceptibility to infections and lower efficacy of vaccination in the elderly population (16-18). Given the multitude of age-associated alterations in the function of the immune system, with some of the most profound occurring in T cells subsets (18, 19), it was hypothesized that age may have an impact on the cell signaling responses measured in this study.

The results shown here demonstrate that some of the variation in healthy immune signaling responses can in fact be attributed to donor demographic characteristics such as age or race. Specifically, the analysis provided herein of the impact of age on immune signaling responses has revealed 4 individual signaling nodes with significant associations with age. Strikingly, all 4 of the individual age-associated immune signaling responses identified here were within naïve T cells, a cell type which has been previously reported to undergo age-related functional changes such as reduced proliferation and cytokine production (18).

The majority (3 of 4) of the individual age-associated signaling nodes confirmed in the PCA analysis and with statistical significance in both training and test sets occurred within the naïve cytotoxic T cell subset, while only 1 of the 4 resided in the naive helper T cell subset. One of the most dramatic age-related changes in the cytotoxic T cell subset is a decrease in the frequency of naïve cytotoxic T cells with age (19, 20), and this was also observed in the samples analyzed in this study (data not shown). Additionally, we have observed an age-related decline in JAK-STAT signaling activity in the naïve cytotoxic T cell subset in response to multiple cytokines including IFNα, IL4, and IL27 (Table 5). Signaling elicited by these cytokines plays a role in cytotoxic T cell survival, proliferation and differentiation (21-24). Thus, the observed age-related decrease in responsiveness to these cytokines may underly some of the functional changes within the cytotoxic T cell compartment. For example, loss of the costimulatory receptor CD28 occurs frequently with increasing age (19) and the resultant CD28-cytotoxic T cells show reduced proliferation, resistance to apoptosis, and higher expression of effector proteins. In addition, a high frequency of CD28-cytotoxic T cells has been shown to correlate with decreased responses to vaccination (25).

The single naïve helper T cell age-associated signaling node was an increased IL2-induced activation of Stat5 (Table 5). This signaling pathway is required for T cell proliferation and activation (26, 27), and both the production of IL2 and the proliferation of naïve helper T cells have been shown to decrease with age (28). The data reported here suggest that the use of IL2 can be an effective strategy for rescuing naïve helper T cell proliferation in the elderly.

Overall, the results reported here provide evidence of age-associated alterations in T cell cytokine signaling responses, with the most striking differences occurring specifically within the naïve cytotoxic T cell subset. While age-associated differences in T cell signaling through the TCR have been widely reported (29), relatively few studies have documented age-related differences in human T cell cytokine signaling (30). Further, much of the work that has been conducted to examine associations between T cell cytokine signaling responses and age has been performed using isolated T cells with techniques such as Western blot analysis that allow for only population-level measurements of pathway activation. Analyses performed at the level of total T cells may fail to capture age-associated alterations specific to a given T cell subset.

The age-associated naïve T cell cytokine signaling responses identified here can play a role in age-related increase in susceptibility to infection, decline in vaccine responsiveness, and the prevalence of certain autoimmune diseases.

Differences in signaling between AAs and EAs, the two major ethnic groups with sufficient representation in this study cohort for statistical analysis, were examined. Because ethnic-related differences have been reported in the prevalence of autoimmune diseases such as systemic lupus erythematosus (31) and multiple sclerosis (32) and in response rates to immunotherapies such as IFNα (10), Benlysta/belimumab (11), and stem cell transplantation (12), it was hypothesized that some of the variation in immune signaling responses may be attributable to racial differences among the study donors. Our assessment of race-associated signaling responses revealed that BCR- (α-IgD) induced PI3K pathway activity was significantly higher in EAs than in AAs. While BCR crosslinking can lead to the activation of multiple signaling pathways, BCR-mediated activation of the PI3K pathway has been shown to provide signaling that plays a role in B cell survival (33). Thus, the differences in PI3K pathway activity observed here can result in racial differences in B cell fate in response to BCR stimulation.

Controlling for ethnicity is emerging as a key component in assuring the accuracy of clinical diagnostics (34) and in selecting treatments (11). For example, AAs and EAs infected with hepatitis C virus have been shown to differ in their response rates to IFNα-based therapy (35) and this has been shown to correlate with in vitro IFNα response profiles (36).

This work demonstrated the utility of the SCNP technology in providing a systems-level description of immune signaling responses within interdependent immune cell subpopulations. Applying this approach to the characterization of immune cell signaling in a cohort of healthy donors allowed for the quantification of the range of signaling across donors and revealed tight ranges for the immune signaling responses measured suggesting that the activation of these signaling nodes can be highly regulated in healthy individuals. Although inter-subject differences in immune signaling responses were generally quite low, within the subset of nodes that displayed the most substantial inter-donor variation some of the variation in immune signaling pathway activation could be attributed to differences in demographic factors such as age or race. Overall, the healthy immune cell signaling network map generated here provides a reference for comparison with network maps generated under disease-associated conditions, using samples from patients at baseline or over the course of therapeutic intervention to identify immune network restructuring that is thought to occur under therapeutic pressure and to guide therapeutic selection.

### REFERENCES

1. Schubert, C. 2011. Systems immunology: complexity captured. Nature 473: 113-114.
2. Irish, J. M., R. Hovland, P. O. Krutzik, O. D. Perez, Ø. Bruserud, B. T. Gjertsen, and G. P. Nolan.

2004. Single cell profiling of potentiated phospho-protein networks in cancer cells. Cell 118: 217-228.
3. Butcher, E. C., E. L. Berg, and E. J. Kunkel. 2004. Systems biology in drug discovery. Nat. Biotechnol 22: 1253-1259.
4. Pos, Z., S. Selleri, T. L. Spivey, J. K. Wang, H. Liu, A. Worschech, M. Sabatino, A. Monaco, S. F. Leitman, A. Falus, E. Wang, H. J. Alter, and F. M. Marincola. 2010. Genomic scale analysis of racial impact on response to IFN-alpha. Proc. Natl. Acad. Sci. U.S.A. 107: 803-808.
5. Krutzik, P. O., and G. P. Nolan. 2003. Intracellular phospho-protein staining techniques for flow cytometry: monitoring single cell signaling events. Cytometry A 55: 61-70.
6. Purvis, N., and G. Stelzer. 1998. Multi-platform, multi-site instrumentation and reagent standardization. Cytometry 33: 156-165.
7. Wang, L., A. K. Gaigalas, and M. Yan. 2011. Quantitative fluorescence measurements with multicolor flow cytometry. Methods Mol. Biol 699: 53-65.
8. Jolliffe, I. T. 2002. Principal Components Analysis, Springer, NY.
9. Dmitrienko, A., and A. C. Tamhane. 2007. Gatekeeping procedures with clinical trial applications. Pharm Stat 6: 171-180.
10. Ge, D., J. Fellay, A. J. Thompson, J. S. Simon, K. V. Shianna, T. J. Urban, E. L. Heinzen, P. Qiu, A. H. Bertelsen, A. J. Muir, M. Sulkowski, J. G. McHutchison, and D. B. Goldstein. 2009. Genetic variation in IL28B predicts hepatitis C treatment-induced viral clearance. Nature 461: 399-401.
11. Ratner, M. 2011. Benlysta makes history. Nat. Biotechnol 29: 298.
12. Mielcarek, M., T. Gooley, P. J. Martin, T. R. Chauncey, B. A. Young, R. Storb, and B. Torok-Storb. 2005. Effects of race on survival after stem cell transplantation. Biol. Blood Marrow Transplant 11: 231-239.
13. Larangé, A., D. Antonios, M. Pallardy, and S. Kerdine-Römer. 2009. TLR7 and TLR8 agonists trigger different signaling pathways for human dendritic cell maturation. J. Leukoc. Biol 85: 673-683.
14. Hall, J. C., and A. Rosen. 2010. Type I interferons: crucial participants in disease amplification in autoimmunity. Nat Rev Rheumatol 6: 40-49.
15. van Boxel-Dezaire, A. H. H., and G. R. Stark. 2007. Cell type-specific signaling in response to interferon-gamma. Curr. Top. Microbiol. Immunol 316: 119-154.
16. Linton, P. J., and K. Dorshkind. 2004. Age-related changes in lymphocyte development and function. Nat. Immunol 5: 133-139.
17. Weng, N.-P. 2006. Aging of the immune system: how much can the adaptive immune system adapt? Immunity 24: 495-499.
18. Haynes, L., and S. L. Swain. 2006. Why aging T cells fail: implications for vaccination. Immunity 24: 663-666.
19. Czesnikiewicz-Guzik, M., W.-W. Lee, D. Cui, Y. Hiruma, D. L. Lamar, Z.-Z. Yang, J. G. Ouslander, C. M. Weyand, and J. J. Goronzy. 2008. T cell subset-specific susceptibility to aging. Clin. Immunol 127: 107-118.
20. Fagnoni, F. F., R. Vescovini, G. Passeri, G. Bologna, M. Pedrazzoni, G. Lavagetto, A. Casti, C. Franceschi, M. Passeri, and P. Sansoni. 2000. Shortage of circulating naive CD8(+) T cells provides new insights on immunodeficiency in aging. Blood 95: 2860-2868.
21. Nelms, K., A. D. Keegan, J. Zamorano, J. J. Ryan, and W. E. Paul. 1999. The IL-4 receptor: signaling mechanisms and biologic functions. Annu. Rev. Immunol 17: 701-738.
22. Curtsinger, J. M., J. O. Valenzuela, P. Agarwal, D. Lins, and M. F. Mescher. 2005. Type I IFNs provide a third signal to CD8 T cells to stimulate clonal expansion and differentiation. J. Immunol 174: 4465-4469.
23. Kolumam, G. A., S. Thomas, L. J. Thompson, J. Sprent, and K. Murali-Krishna. 2005. Type I interferons act directly on CD8 T cells to allow clonal expansion and memory formation in response to viral infection. J. Exp. Med 202: 637-650.
24. Morishima, N., I. Mizoguchi, M. Okumura, Y. Chiba, M. Xu, M. Shimizu, M. Matsui, J. Mizuguchi, and T. Yoshimoto. 2010. A pivotal role for interleukin-27 in CD8+ T cell functions and generation of cytotoxic T lymphocytes. J. Biomed. Biotechnol 2010: 605483.
25. Effros, R. B. 2007. Telomerase induction in T cells: a cure for aging and disease? Exp. Gerontol 42: 416-420.
26. Lin, J. X., and W. J. Leonard. 2000. The role of Stat5a and Stat5b in signaling by IL-2 family cytokines. Oncogene 19: 2566-2576.
27. Moriggl, R., D. J. Topham, S. Teglund, V. Sexl, C. McKay, D. Wang, A. Hoffmeyer, J. van Deursen, M. Y. Sangster, K. D. Bunting, G. C. Grosveld, and J. N. Ihle. 1999. Stat5 is required for IL-2-induced cell cycle progression of peripheral T cells. Immunity 10: 249-259.
28. Linton, P. J., L. Haynes, N. R. Klinman, and S. L. Swain. 1996. Antigen-independent changes in naive CD4 T cells with aging. J. Exp. Med 184: 1891-1900.
29. Fülöp, T., Jr, A. Larbi, G. Dupuis, and G. Pawelec. 2003. Ageing, autoimmunity and arthritis: Perturbations of TCR signal transduction pathways with ageing—a biochemical paradigm for the ageing immune system. Arthritis Res. Ther 5: 290-302.
30. Fulop, T., A. Larbi, N. Douziech, I. Levesque, A. Varin, and G. Herbein. 2006. Cytokine receptor signalling and aging. Mech. Ageing Dev 127: 526-537.
31. McCarty, D. J., S. Manzi, T. A. Medsger Jr, R. Ramsey-Goldman, R. E. LaPorte, and C. K. Kwoh. 1995. Incidence of systemic lupus erythematosus. Race and gender differences. Arthritis Rheum 38: 1260-1270.
32. Rosati, G. 2001. The prevalence of multiple sclerosis in the world: an update. Neurol. Sci 22: 117-139.
33. Srinivasan, L., Y. Sasaki, D. P. Calado, B. Zhang, J. H. Paik, R. A. DePinho, J. L. Kutok, J. F. Kearney, K. L. Otipoby, and K. Rajewsky. 2009. PI3 kinase signals BCR-dependent mature B cell survival. Cell 139: 573-586.
34. Moore, B., H. Hu, M. Singleton, M. G. Reese, F. M. De La Vega, and M. Yandell. 2011. Global analysis of disease-related DNA sequence variation in 10 healthy individuals: implications for whole genome-based clinical diagnostics. Genet. Med 13: 210-217.
35. Reddy, K. R., J. H. Hoofnagle, M. J. Tong, W. M. Lee, P. Pockros, E. J. Heathcote, D. Albert, and T. Joh. 1999. Racial differences in responses to therapy with interferon in chronic hepatitis C. Consensus Interferon Study Group. Hepatology 30: 787-793.
36. He, X.-S., X. Ji, M. B. Hale, R. Cheung, A. Ahmed, Y. Guo, G. P. Nolan, L. M. Pfeffer, T. L. Wright, N. Risch, R. Tibshirani, and H. B. Greenberg. 2006. Global transcriptional response to interferon is a determinant of HCV treatment outcome and is modified by race. Hepatology 44: 352-359.

### Example 5

Overview:

Given the biologic and clinical heterogeneity inherent to AML, an unmet medical need exists for tools to guide the choice of drugs most relevant to the underlying biology of the individual AML. Single Cell Network Profiling (SCNP) can be used as a tool to inform biology-based clinical decision making including therapy selection and disease monitoring. Previous studies have provided preliminary proof-of-concept on the utility of SCNP to dissect the pathophysiologic heterogeneity of hematologic tumors and assess their differential response to single agent and combination therapies. This study characterizes the signal transduction networks implicated in the growth and survival of AML cells and how those are affected by in vitro exposure to various FDA-approved and investigational therapeutic agents. Compounds were selected based on their ability to disrupt key mechanisms of AML tumor cell growth and survival.

Design:

This study used peripheral blood or bone marrow samples (n=9), which had been previously ficoll separated and cryopreserved. Patient characteristics are shown Table 7. One cryovial per patient was used. Samples were thawed and centrifuged over ficoll to remove dead cells and debris.

TABLE 7
Patient Characterisics

| Reference | Disease | Sample Timepoint | Receipt date | Age | Usage |
|---|---|---|---|---|---|
| 1910-006 | AML | Pre-induction | Dec. 2, 2010 | 36 | 1 vial (10 million cells) |
| 1910-008 | AML | Post-induction | Dec. 2, 2010 | 47 | 1 vial (10 million cells) |
| | | Resistant | | | |
| 1910-011 | AML | Post-induction | Feb. 18, 2011 | 52 | 1 vial (10 million cells) |
| | | Resistant | | | |
| 1910-013 | AML | Relapse On Therapy | Jan. 15, 2011 | 60 | 1 vial (10 million cells) |
| 1910-015 | AML | Pre-induction | Jan. 19, 2011 | 83 | 1 vial (10 million cells) |
| 1910-016 | AML | Post-induction | Feb. 23, 2011 | 37 | 1 vial (10 million cells) |
| | | Resistant | | | |
| 1910-017 | AML | Pre-induction | Feb. 9, 2011 | 71 | 1 vial (10 million cells) |
| 1910-018 | AML | Relapse Off Therapy | Feb. 10, 2011 | 66 | 1 vial (10 million cells) |
| 1910-019 | AML | Pre-Induction | Apr. 13, 2011 | 24 | 1 vial (10 million cells) |

Samples were split to perform the following:

Arm #1 assessed basal and modulated signaling in the JAK/STAT, PI3K/mTor, and MEK/ERK pathways in the presence and absence of specific kinase inhibitors. Kinase inhibitors were added 1 hr before the addition of the signaling stimulus. Signaling was induced by individual addition of stem cell factor, Flt3 ligand, G-CSF, IL-3, or thrombopoietin (TPO) for a short period of time (5-15 min). Cells were then fixed, permeabilized, and stained with a cocktail of cell surface and phospho-specific antibodies to measure signaling in multiple cell types. Signaling data is calculated in each cell type using a fold-change metric comparing each condition to its basal state: example: (stimulated^{+/- inhibitor})/(unstimulated). Also, cells with an apoptotic phenotype were excluded from the signaling analysis by gating.

Arm #2 assessed the cytotoxic and cytostatic impact of various drugs as single agents and in combinations (including the specific kinase inhibitors tested in arm #1). Here the cells from each donor were cultured in the presence of TPO, IL-3, SCF, and FLT3L for 2 days to drive proliferation. After 2 days the cells were then distributed into wells containing various drugs, wherein the cells were cultured for 48 hours. The cultures were fixed, permeabilized, and stained with a cocktail of antibodies to measure complete cell death, apoptosis, S/G2 phase, M-Phase, and DNA damage. These readouts were also obtained from samples cultured separately with individual growth factors (no drugs) for 4 days.

A schematic of the experiment is shown in FIG. 3.

Examples of reports for a subject (#1910-017) are shown in FIGS. 8, 9, and 10. In FIG. 8A, a cell lineage diagram is depicted. Percentages of cell types are show for subject #1910-017 (circle on graph, e.g., see FIG. 8B) and for healthy or normal cells (bar on graph). The report depicts fold activation of activatable elements relative to a basal state in radar plot form to allow comparison of the subject sample with fold activation ranges for normal samples (see e.g., FIG. 8B). Fold activation is indicated for samples that were or were not contacted with a kinase inhibitor. FIGS. 8B, 8C, 8D, and 8E show information for different cell types

Another form of a report is depicted in FIG. 9. FIG. 9A indicates percentages of cells in a ring diagram. The outer circle corresponds to cells in the #1910-017 AML sample of PBMCs pre-induction. The inner circle corresponds to percentages of cells in healthy bone marrow. The percentages do not add up to 100%, as some types cells are not included. Fold change from basal state of cell signaling is indicated as a heat map.

For CD34+ cells, patient #1910-017 has high basal p-AKT level that is attenuated by PI3K/mTor inhibitor, but not FLT3 inhibitor. This suggests that the high basal level is not a function of high FLT3 activity. There is also a high p-STAT5 basal level. There is no FLT3L or G-CSF responses, which are observed in healthy CD34+ cells. The CD34-CD117+ cell population has a similar signaling phenotype as the CD34+ cells. The CD34-CD117-cells respond strongly to TPO, but not to G-CSF. The lymphocytes have no signaling. High basal level of p-STAT5 signaling is inhibited by CP-690550.

The report indicates drug responses. The response to AC220 is not known due to no FLT3L induced signaling in #1910-017. With respect to GDC-0941, there is partial inhibition of SCF-pAKT and pS6. With respect to AZD-6244, there is complete inhibition of SCF-pERK, partial inhibition of pS6, and no inhibition of pAKT. With respect to BEZ235, there is complete inhibition of SCF induced pAKT, and partial inhibition of pS6. With respect to CP-690550, there is complete inhibition of IL-3 signaling, and partial inhibition of TPO signaling.

FIG. 9D shows growth factor dependent effects on cell growth and survival. Survival and cell growth appear independent of growth factor stimulation.

FIGS. 9D and 9E show drug induced apoptosis and cytostasis. In general, this patient's myeloid cells resisted apoptosis for most drugs, including AraC. However, inhibition of cell cycle (M-phase) was observed for many drugs. Proteosome inhibition (bortezomib) induced considerable levels of cell death and cytostasis. HSP90 inhibitor also induced apoptosis.

FIG. 10 shows another example of a report for a subject (#1910-017). FIG. 10 illustrates information on percentage of cell types (based on surface phenotype) in a sample from the subject and percentages of cell types in normal or healthy cells (see e.g., FIG. 10G). FIG. 10 contains biological information on the cell types (see e.g., FIG. 10B). Information on signaling phenotypes are illustrated as radar plots (see e.g., FIGS. 10C, 10D, 10E, and 10F). The report in FIG. 10 also contains information on cell growth and cell survival and cytostasis after drug exposure.

### Example 6

### Healthy Bone Marrow FLT3 Pathway Signaling

Healthy bone marrow myeoblasts (BMMb) display similar FLT3L induced signaling while AML samples display a range of responses. These data allow for comparison of leukemic to healthy responses.

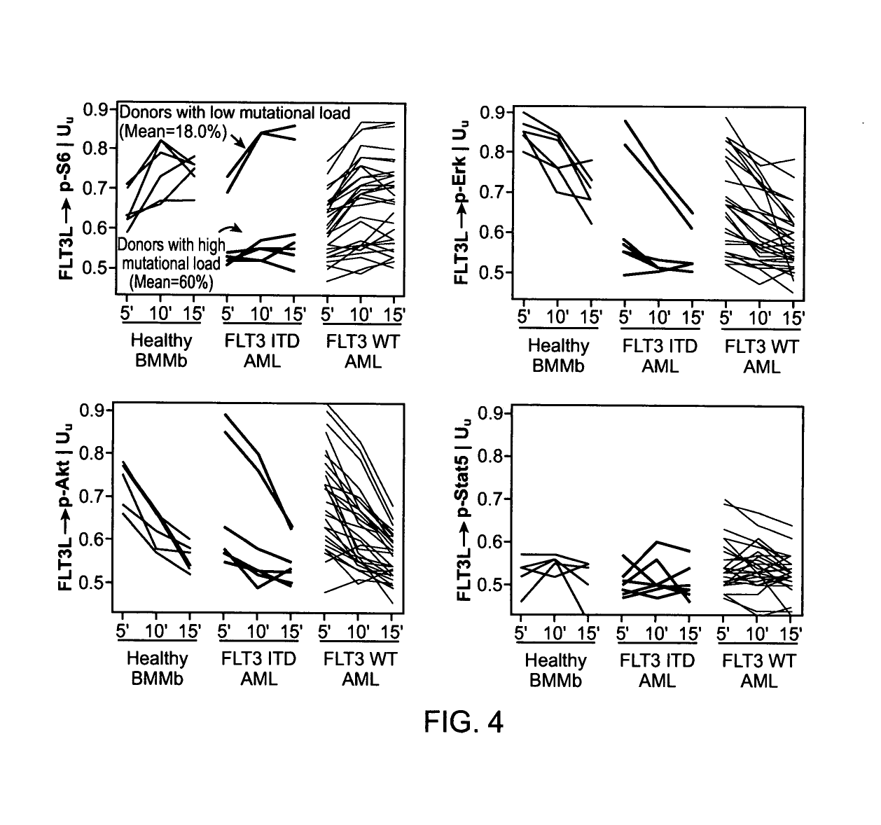

FLT3 ligand induced signaling of p-S6, p-Erk, p-Akt, and p-Stat5 at 5, 10, and 15 min time points in healthy bone marrow myeloblats (BMMb), and leukemic blasts from AML donors with or without FLT3-ITD (internal tandem duplication) mutation are shown in FIG. 4. FLT3-ITD AML with high mutational load responses are more homogenous than FLT3-WT AML (FIG. 4).

A PCA (principal component analysis) of healthy BMMB, FLT3-TD, and FLT3-WT samples illustrate homogeneity of BMMB and FLT3-ITD mutated samples and heterogeneity of FLT3-WT samples. Distinct signaling patterns were seen among groups.

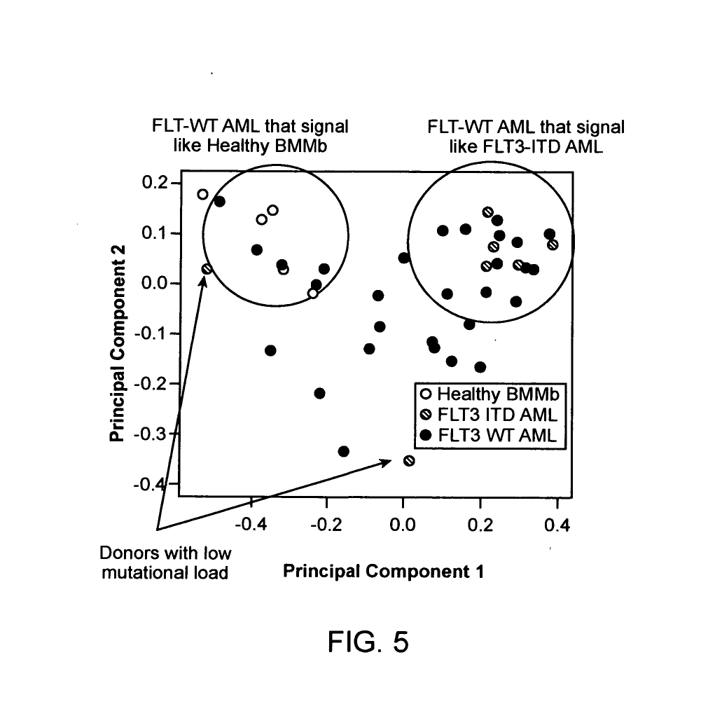

FLT3 WT donors are more heterogeneous than FLT3 ITD donors and show distinct patterns. Some signal like Healthy BMMb; some signal like FLT3-ITD AML; some signal like neither group. Donors with low mutational load stand out from FLT3-ITD group. Comparison of AML to Healthy BMMb identifies AML donors that behave similar to or distinct from Healthy BMMb. (see FIG. 5)

### Example 7

### Impact of Time from Blood Draw to Peripheral Blood Mononuclear Cell (PBMC) Processing and Cryopreservation on Functional Pathway Activity as Measured by Single Cell Network Profiling (SCNP) Assays

Cryopreserved peripheral blood mononuclear cells (PBMCs) can be routinely used in biomarker development studies. Multiple pre-analytic parameters related to blood draw, processing, and cryopreservation can impact the quality of PBMC samples used in functional assays. Single cell network profiling (SCNP) can be a multi-parametric flow cytometry based approach that can measure intracellular signaling activity in response to extracellular modulators. Preservation of cell viability and functionality plays a role in the performance of the SCNP assay. In other immunological assays, such as the ELISpot assay, the length of time from blood draw to PBMC cryopreservation can affect assay performance. In this study, the effect of time from sample collection to cryopreservation on functional pathway activation was assessed by comparing SCNP assay readouts in paired PBMC samples processed within 8 or 32 hrs from blood draw.

Forty mLs of peripheral blood was obtained for 20 donors (10 male/10 female, 60-83 yrs) at the Stanford Blood Center. Half of the sample volume from each donor was processed within 8 hrs of blood draw [Day 1 (D1)], and the remainder left at 25° C. overnight [Day 2 (D2)]. For D2 samples, PBMC isolation and cryopreservation were initiated 24 hrs from the processing start time of the corresponding D1 sample. For the SCNP assay, samples were thawed, modulated for 15 mins with 12 immunomodulatory stimuli (interferons, interleukins, TLR ligands, etc.), fixed, and permeabilized. Permeabilized cells were stained with fluorochrome-conjugated antibodies recognizing extracellular surface markers or intracellular signaling molecules (pStat1, pStat3, pStat5, pS6, pNFκB, pAkt, and pErk). Thirty eight signaling nodes (readouts of modulated signaling) were measured in 7 distinct immune cell subsets (monocytes, B cells, NK cells, naive/memory helper T cells, and naive/memory cytotoxic T cells).

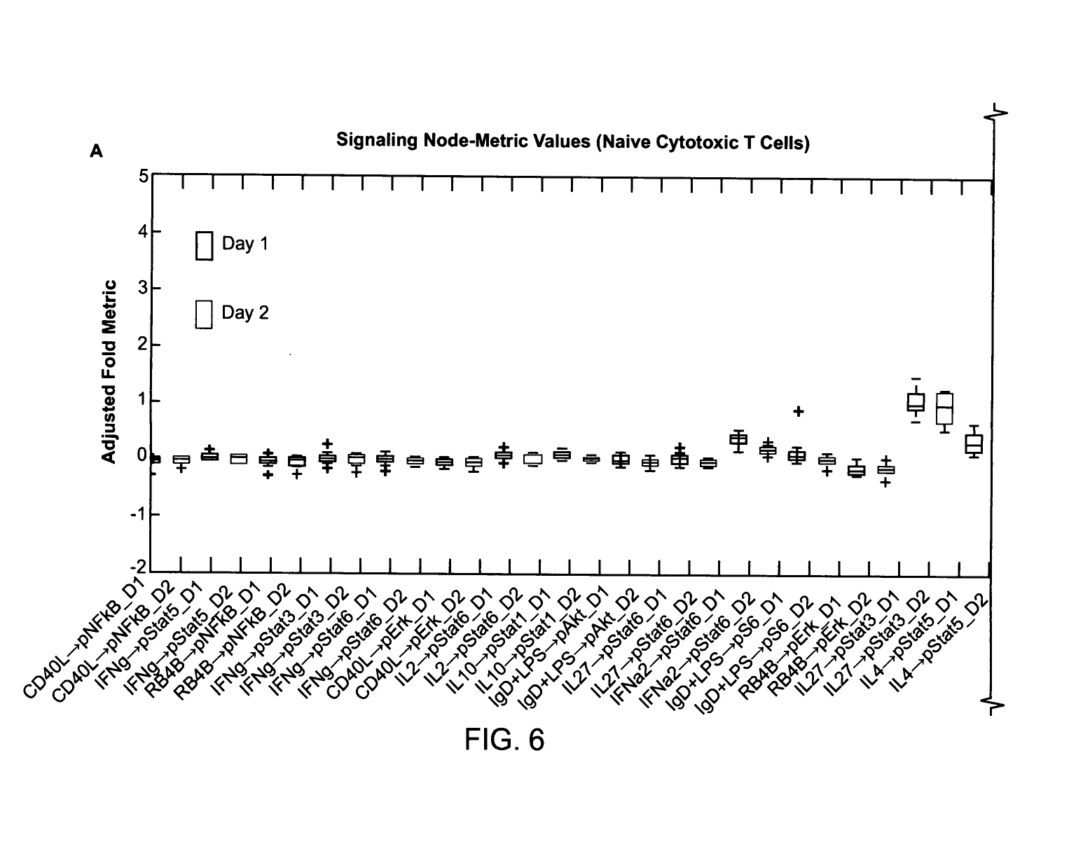

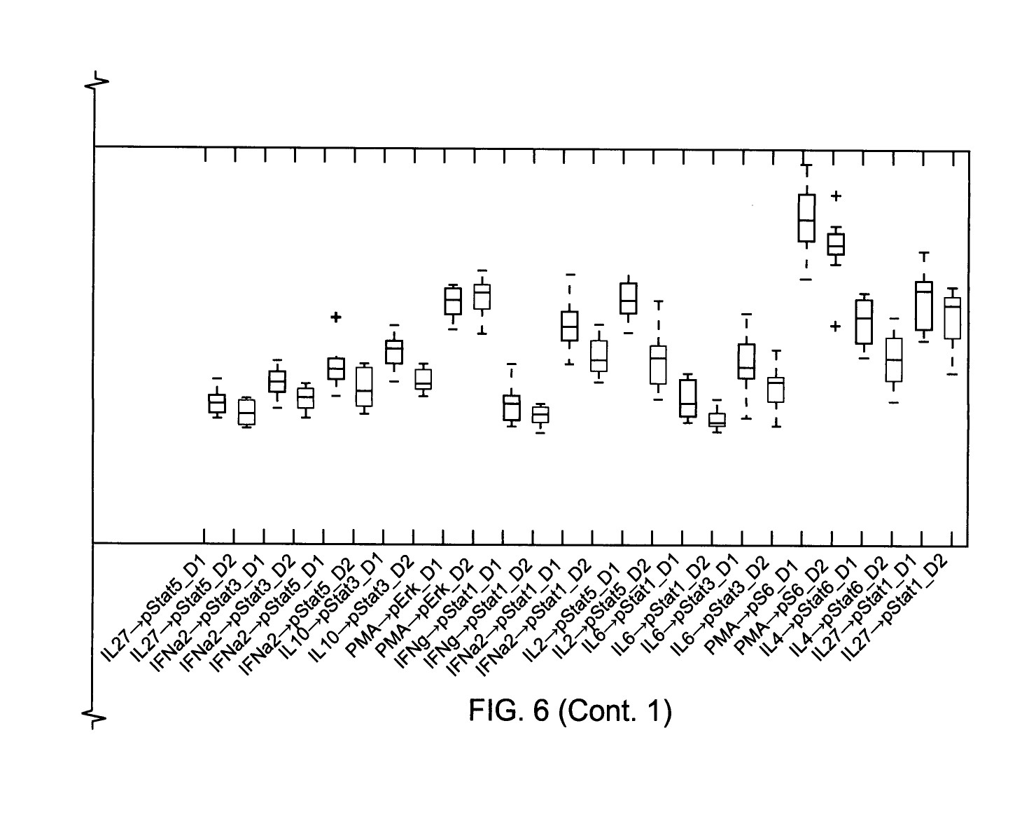

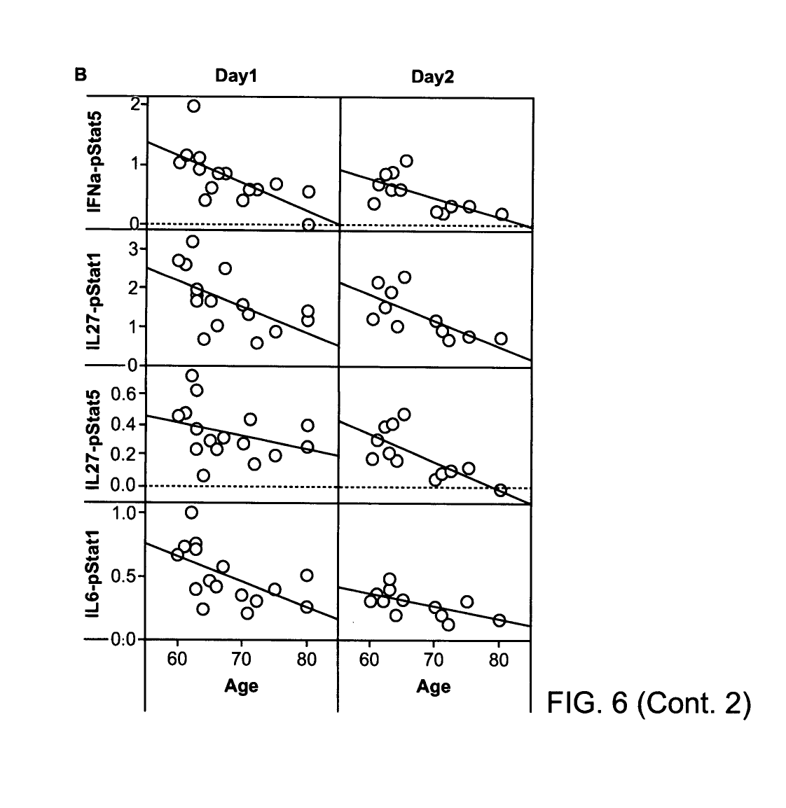

Analysis of paired PBMC samples revealed that D1 and D2 samples had no significant difference in the percentage of healthy cells (measured by the percentage of cleaved PARP″ cells) and no difference in subpopulation frequencies (as a percentage of parent populations) for the majority of the 7 subsets examined. A numerically small but statistically significant decrease in the percentage of healthy cells in D2 compared to D1 samples was observed for B cells, NK cells, and naive helper T cells (mean difference 5.6%, 5.8%, and 3.2% respectively, p<0.05) while the monocyte subset was the only one to show a significant decrease (9.4%, p<0.05) in frequency (as a percentage of parent) on D2. Similar intracellular signaling pathway modulation responses were observed for D1 and D2 samples (FIG. 6A), although the majority of nodes displayed lower modulated responses in D2 samples (10.0% mean decrease between D1 and D2). A good correlation (Spearman r>0.5) between D1 and D2 was observed for the majority (63%) of responsive signaling nodes. Within each dataset, inter-node correlation coefficients were calculated to generate immune signaling network maps. Comparing these maps showed good agreement between the correlations measured within each dataset [mean difference of −0.01 between inter-node correlations across days (D1 mean correlation 0.21, D2 mean correlation 0.20)] demonstrating biological consistency between the 2 datasets in the structure of the immune signaling network. Further, 13 age-associated differences (p<0.05) in immune signaling responses were identified in the D1 dataset and the majority of these remained significant in the D2 dataset (p<0.05). For example, several cytokine signaling responses within naïve cytotoxic T cells had a significant decrease with age in both datasets (FIG. 6B).

These results demonstrate that blood samples processed the day following blood draw provide meaningful information on functional pathway activation using the SCNP assay and support the identification of statistically significant associations with clinical variables such as age. In a clinical setting, overnight shipping of patient samples to the lab performing the test may be required.

### Example 8

### Stimulus-Specific and Cell-Subset-Specific Inter-Donor Variation in Immunological Signaling Responses in Healthy Individuals

Single cell network profiling (SCNP) can be a multi-parameter flow cytometry based approach that can allow for the simultaneous interrogation of intracellular signaling pathways in multiple cell subpopulations within heterogeneous tissues such as peripheral blood or bone marrow. The SCNP approach is well-suited for characterizing the multitude of interconnected signaling pathways and immune cell subpopulations that interact to regulate the function of the immune system. Recently, SCNP was applied to generate a functional map of the “normal” human immune cell signaling network by profiling immune signaling pathways downstream of a broad panel of immunomodulators in multiple immune cell subsets within peripheral blood mononuclear cells (PBMCs) from a large cohort of healthy donors. In this study, an in-depth analysis of the inter-donor variation in normal immune signaling responses was performed. This analysis demonstrated that the degree of inter-donor variation in immune signaling responses does not vary directly with the magnitude of the response. Instead, cell subpopulation-specificity and stimulus-specificity in the degree of inter-donor response heterogeneity was observed. Further, an analysis of variation in signaling activity at the single cell level revealed that inter-donor variation in immune signaling responses may arise primarily due to donor-to-donor differences in the proportion of responding cells or, alternatively, due to inter-donor differences in the intensity of the response from relatively homogeneously responding subpopulations. The characterization of normal inter-donor variation in immune signaling pathway activation presented here provides a basis for identifying immune signaling abnormalities in immune-mediated diseases.

Introduction

The human immune system is composed of a complex network of cell types and signaling pathways that, in healthy individuals, can interact to provide immunity against pathogens and tumor-associated antigens while simultaneously preventing detrimental immune responses to self-antigen. Deregulation of immune cell signaling network responses can result in aberrant immune function leading to increased susceptibility to diseases such as autoimmunity, chronic infections, and cancer. Because immune responses can be governed by a network of distinct cell types, systems-level analyses that measure the activity of intracellular signaling networks within multiple immune cell types can provide more clinically relevant insight into the basis of immune-mediated disorders and the effects of therapeutic intervention on the function of the overall immune system than traditional immunological studies which focus on the behavior of a specific immune cell subset following isolation from complex tissues such as peripheral blood, lymph nodes, or the spleen.

Single cell network profiling (SCNP) is a flow-cytometry based approach that is well-suited for investigating how the immune system responds and reacts to external stimuli at a network-level, because the SCNP approach can allow for the simultaneous interrogation of modulated signaling activity across multiple signaling pathways in multiple interdependent immune cell subpopulations. The SCNP technology has been applied extensively to disease characterization and patient stratification in hematological malignancies such as acute myeloid leukemia (AML) and chronic lymphocytic leukemia (CLL) (1-3).

More recently, SCNP technology was applied to generate a functional map of “normal” human immune signaling responses to provide a reference for identifying signaling abnormalities in pathological conditions such as autoimmunity. To generate the “normal” immune signaling network map, SCNP was used to profile signaling pathways downstream of a broad panel of immunomodulators (including interferons, interleukins, IgD crosslinking, TLR ligands, and CD40L) in seven distinct, non-sorted immune cell subpopulations within peripheral blood mononuclear cells (PBMCs) from a large cohort of healthy individuals (see Example 4). While the majority of the immune signaling nodes measured in the “normal” immune signaling network mapping displayed a relatively narrow range of responses across the cohort of healthy donors, a subset of the immune signaling responses displayed considerable inter-donor variation.

A greater understanding of the degree of donor-to donor variation in immune signaling responses across healthy donors can be used to determine which immune signaling responses in cells from diseased donors can be classified as abnormal. Establishing inter-donor variation in immune signaling responses from healthy individuals that can be attributed to differences in demographic factors such as age, race, or gender can provide insight into the basis for disparities in the prevalence of immune-mediated disease among different donor subgroups. Notably, some of the inter-donor variation in immune signaling responses surveyed in the “normal” immune signaling mapping can be attributed to differences in demographic factors such as age and race.

Here, an in-depth analysis of the degree of inter-donor variation in immune signaling network responses was performed to assess patterns in the distribution of signaling nodes which displayed high heterogeneity across the healthy donor cohort. This analysis revealed that the degree of inter-donor variation did not vary directly with the magnitude of the response. In addition, high inter-donor variation was not restricted to a specific cell type or modulator. Instead, the level of inter-donor heterogeneity in the activation of a given signaling molecule was dependent both on the stimuli used to modulate the signaling protein and on the immune cell subpopulation in which the signaling molecule was activated. Further, this study demonstrated that inter-donor heterogeneity in modulated signaling activity from a given signaling component within a specific cellular subpopulation can be driven by a uniform subpopulation response of differing intensities across donors, or alternatively, can arise due to differences in the frequency of responsive cells (subpopulation heterogeneity) across the donors. These findings have implications for the characterization of immune signaling abnormalities in pathological conditions such as autoimmunity and cancer.

Results

Global Analysis of Inter-Donor Variation

Intracellular signaling activity across multiple immune cell subpopulations was analyzed using single cell network profiling (SCNP) as described in Example 4. The phosphorylation status of 8 signaling proteins (Stat1, Stat3, Stat5, Stat6, Akt, S6, Erk, and NFκB) was measured in response to 12 stimuli (IFNα, IFNγ, IL2, IL4, IL6, IL10, IL27, α-IgD, LPS, R848, PMA, and CD40L) in seven distinct (non-overlapping) immune cell subpopulations (monocytes, B cells, CD3-CD20-lymphocytes (natural killer cell-enriched subpopulation), naïve helper T cells, memory helper T cells, naïve cytotoxic T cells, and memory cytotoxic T cells) within unsorted PBMC samples from 60 healthy individuals. The Fold metric (Materials and Methods) was utilized to measure the levels of intracellular signaling proteins in response to modulation, and the interquartile range (IQR) for the Fold was used to quantify the degree of inter-donor variation for each signaling node (readout of modulated signaling, see Materials and Methods) in each immune cell subpopulation.

A global analysis of the inter-donor variation in immune signaling responses was performed by determining which signaling responses displayed relatively high inter-donor variation using the average IQR (0.03) as a threshold. Notably, all of the signaling responses that displayed modulated activity above a threshold of Fold >0.25 (representing an approximately 1.2 fold change in modulated levels relative to basal levels, see Materials and Methods). Thus, perturbing the immune signaling network allows for the detection of donor-to-donor heterogeneity that is more substantial than the inter-donor heterogeneity that is observed from the unperturbed network.

Although high inter-donor hetereogeneity was confined to signaling responses that showed a response above the 0.25 Fold threshold value, the degree of inter-donor variation did not vary directly with the magnitude of the response. Thus, it was sought to determine if high inter-donor variation was restricted to specific immune cell subpopulations and/or to responses to specific immunomodulators. For each of the cell subpopulations, the percentage of responsive signaling nodes that showed high inter-donor variation was calculated. This analysis revealed that, of the signaling nodes that modulated, a greater percentage of these signaling responses showed high inter-donor heterogeneity in the T cell subpopulations and CD3-CD20-lymphocytes than in the monocytes and B cells. Next, to assess which modulators produced responses with high inter-donor variation, the percentage of responsive signaling nodes that that showed high inter-donor variation was determined for each stimulus. For a few of the modulators, such as BCR/LPS, PMA, and IL2, all or most of the responses displayed high inter-donor variation. However, for the majority of the modulators, the degree of inter-donor variation in the responses differed amongst the different cell subsets and amongst the different phospho-protein readouts. For example, modulation with IFNγ resulted in pStat1 responses with high inter-donor variation in monocytes and B cells, but not in the naïve T cell subsets, and IFNγ-induced pStat3 and pStat5 showed low inter-donor variation in monocytes unlike the IFNγ-induced p-Stat1 responses in this subpopulation.

Stimulus-Specific Inter-Donor Variation in Immune Signaling

As discussed previously, the IQR did not vary directly with Fold across the full panel of signaling nodes measured in all of the immune subpopulations. Thus, it was next investigated whether there was a direct relationship between Fold and the IQR for responses by a specific phospho-protein readout within a given immune subpopulation across multiple stimuli. Inter-donor variation in pStat1 signaling did not vary directly with the magnitude of the pStat1 response, but instead displayed stimulus-specificity. To assess the validity of this observation, the values of the Fold and the degree of inter-donor variation for half of the donors randomly assigned to a training set were compared with the values for the second half of the donors assigned to a test set (Materials and Methods). The values were remarkably consistent across both donor sets confirming the observation of stimulus-specificity in inter-donor hetereogeneity (data not shown).

Cell Subset-Specific Inter-Donor Variation in Immune Signaling

Next, the relationship between the degree of inter-donor heterogeneity and the magnitude of the response for a specific signaling node across multiple cell subpopulations was analyzed. There is not a direct relationship between the degree of inter-donor variation and the magnitude of the pStat5 response (data not shown). In both training and test data sets, the IQR for naïve helper T cells is extremely high despite a relatively moderate Fold for this cell subset (data not shown). In addition, CD3-CD20-lymphocytes, memory cytotoxic T cells, naïve cytotoxic T cells, and memory helper T cells display similar degrees of inter-donor variations despite differences in the intensity of the pStat5 response in each of these subsets. Thus, the inter-donor variation in IL2-induced pStat5 displayed cell-type specificity and did not vary directly with the magnitude of the pStat5 response in each cell type.

Single Cell Analysis Reveals Cell Subpopulation Heterogeneity

Use of flow cytometry can allow for the quantification of immune signaling responses in each of the individual cells in a given population or subpopulation. Notably, the IL2-induced pStat5 responses showed strong bimodality, where a portion of the cells in each subpopulation show elevated pStat5 levels following IL2 treatment while a subset of the cells overlap with the basal pStat5 distribution (data not shown). Interestingly, the frequency of IL2 responsive cells in each of the T cell subpopulations varied from donor to donor. Further, the inter-donor variation in IL2-induced pStat5 Fold values (data not shown) are driven primarily by differences in the proportion of cells that respond to IL2 rather than the intensity of the response in the responsive subset (data not shown). In contrast to the bimodal pStat5 responses observed following IL2 stimulation, the T cell subpopulations displayed unimodal pStat5 levels following stimulation with IFNα. For the IFNα→pStat5 signaling node, the inter-donor differences were determined primarily by the intensity of the pStat5 responses over relatively homogenous subpopulations. Thus, the results shown here demonstrate that inter-donor variation in immune signaling responses can arise due to inter-donor differences in the degree of subpopulation heterogeneity or due to inter-donor differences in the response magnitudes from homogeneously responding subpopulations.

Discussion

Immune responses can be regulated by a complex network of diverse cell types and interconnected signaling pathways. Deregulation of the immune system can lead to dampened immune responses to pathogens and tumor cells (immunodeficiency), excessive immune responses to innocuous foreign antigens (hypersensitivity), or to inappropriate responses to self-antigens (autoimmunity). A greater understanding of the alterations in the immune cell signaling network that underlie immune-mediated diseases can lead to improved methods for diagnosing and treating such diseases. However, determining which immune signaling responses from diseased patients can be classified as abnormal can involve comprehensive knowledge of the immune cell signaling network in the baseline, or disease-free, state. Recently, single cell network profiling (SCNP) was applied to generate a functional map of the normal immune cell signaling network by measuring immune signaling responses to a broad panel of immunomodulators in multiple immune cell subpopulations within PBMCs from a large number of healthy individuals (See Example 4). This “normal” characterization can provide a basis for comparison with diseased specimens to identify, within the immune cell signaling network, which responses differ significantly from the baseline state and which responses are within the normal range of variation.

In this study, the distribution of the degree of inter-donor variation was analyzed in immune signaling responses across the normal immune cell signaling network. The results of this analysis have revealed that immune signaling responses with relatively high inter-donor variation, as quantified by the interquartile range (IQR), are not confined to specific immune cell subsets or to intracellular signaling responses to specific immunomodulators. The immune signaling responses that displayed high inter-donor variation were, however, restricted to the subset of immune signaling responses that showed activation above a relatively low threshold. These results highlight the role of applying a perturbation to probe the functional capacity of the immune system and to reveal donor-to-donor differences in the behavior of the immune cell signaling network.

Although high inter-donor variation was restricted to immune signaling responses that showed some degree of modulated activity, there was not a direct linear relationship between the magnitude of the response and the degree of inter-donor variation for the full panel of immune signaling responses (data not shown). In addition, when the analysis of the relationship between response magnitude and inter-donor variation in the response was restricted to a specific signaling node within a specific immune cell subpopulation, the degree of inter-donor variation, again, did not vary directly with the magnitude of the response. Instead, the degree of inter-donor heterogeneity displayed stimulus-specificity (data not shown). Likewise, narrowing the analysis of the relationship between response magnitude and inter-donor variation in the response to the activation of a specific intracellular protein by a specific modulator revealed cell subpopulation-specificity in the degree of inter-donor variation and, again, a poor correlation between the level of inter-donor variation and the response magnitude (data not shown). Thus, these results demonstrate that the degree of normal human variation in immune signaling is not generalizable for a given protein readout, immunomodulator, or cell subpopulation. These findings have implications for the identification of immune signaling responses that may have utility as clinical biomarkers for diagnosis, prognosis, and treatment selection in immune-mediated pathologies.

Because the SCNP workflow involves measuring signaling activity using flow cytometry, this approach allowed for an investigation of the variation in signaling activity among individual cells within phenotypically defined immune cell subpopulations. An analysis of signaling activity at the single-cell level revealed bimodality in IL2-induced Stat5 phosphorylation, even with relatively well-defined T cell subpopulations (data not shown). This subpopulation heterogeneity in IL2 responsiveness may be driven by variation in the expression of the IL2 receptor. Recent work has shown that expression levels of the IL2R subunits (IL2Rα, IL2Rβ, and IL2Rγ) can vary substantially in clonal T cell populations (4). Thus, considerable variation may be expected for non-clonal T cell subpopulations.

Notably, the frequency of IL2 responsive cells within each subpopulation varied widely from donor to donor with relatively small donor-to-donor differences in the pStat5 intensities for the responsive cells (data not shown). Thus, the high inter-donor variation in the IL2-induced pStat5 Fold values can be due to differences in the frequency of responsive cells. Assessing the inter-donor and intra-subpopulation variations in IL2-induced Stat5 phosphorylation in immune subpopulations within patient samples can have clinical relevance given the use of IL2 as an immunotherapy for the treatment of metastatic melanoma and renal cell carcinoma. Because high dose IL2 therapy can be associated with severe toxicity and only a subset of patients respond to treatment with IL2 (5), the identification of biomarkers for predicting response to IL2 immunotherapy can have high clinical utility.

Subpopulation heterogeneity in signaling activity was also observed following treatment with α-IgD in the B cell subpopulation (data not shown). For this modulator, the presence of responsive and non-responsive B cells was expected due to the lack of IgD expression on a portion of B cells (i.e. immature B cells and class-switched B cells). In contrast to IL2 and α-IgD responses, the majority of the signaling nodes that were profiled in this study displayed relatively homogeneous (unimodal) responses within the seven distinct immune cell subpopulations (data not shown). The observed homogeneity in signaling for many of the immune signaling nodes surveyed here may be reflective of relatively homogeneous expression of the corresponding receptors across each of the seven immune cell subpopulations.

In summary, the degree of normal inter-donor variation in the responsiveness of a given phospho-protein readout can be highly specific to both the immunomodulator used to generate the response and the cell subpopulation in which the response is measured. Quantifying the normal variation in immune signaling responses within the immune cell signaling network can play a role in establishing normal baseline ranges against which diseased specimens can be compared and thus provides a foundation for the discovery of biomarkers that can aid in the diagnosis, treatment selection, and clinical monitoring of diseases such as cancer and autoimmunity.

Materials and Methods

PBMC Samples

Sixty cryopreserved peripheral blood mononuclear cell (PBMC) samples taken from healthy donors within the Department of Transfusion Medicine, Clinical Center, National Institutes of Health were used in this study. Blood donations from healthy donors were collected and processed as described previously (6).

SCNP Assay and Flow Cytometry

The SCNP assay and flow cytometry data acquisition and analysis were performed as previously described (see Example 4). Briefly, cryopreserved PBMC samples were thawed at 37° C. and re-suspended in RPMI 1% FBS before staining with amine aqua viability dye (Invitrogen, Carlsbad, Calif.). Cells were re-suspended in RPMI 1% FBS, aliquoted to 100,000 cells per condition, and rested for 2 hours at 37° C. prior to incubation with modulators at 37° C. for 15 minutes. After exposure to modulators, cells were fixed with paraformaldehyde and permeabilized with 100% ice-cold methanol. Methanol permeabilized cells were washed with FACS buffer (PBS, 0.5% BSA, 0.05% NaN₃), pelleted, and stained with antibody cocktails containing fluorochrome-conjugated antibodies against phenotypic markers for cell population gating and up antibodies against intracellular signaling molecules. Flow cytometry data was acquired using FACS DNA software (BD Biosciences, San Jose, Calif.) on two LSRII Flow Cytometers (BD Biosciences, San Jose, Calif.). Flow cytometry data was analyzed with WinList (Verity House Software, Topsham, Me.). For all analyses, dead cells and debris were excluded by forward scatter (FSC), side scatter (SSC), and amine aqua viability dye. PBMC subpopulations were delineated according to an immunophenotypic gating scheme.

SCNP Terminology and Metrics

The term “signaling node” can refer to a specific protein readout in the presence or absence of a specific modulator. For example, the response to IFNα stimulation can be measured using pStat1 as a readout. This signaling node can be designated “IFNα→pStat1”. Each signaling node can be measured in each cell subpopulation. The cell subpopulation can be noted following the node, e.g., “IFNα→pStat|B cells”. The “Fold” metric can be applied to measure the level of a signaling molecule after modulation compared to its level in the basal state. The Equivalent Number of Reference Fluorophores (ERFs), fluorescence measurements calibrated by rainbow calibration particles, serve as a basis for all metric calculations (7-9).

The “Fold” metric can be calculated as follows:

(log₂[ERF(Modulated)/ERF(Unmodulated)]+Ph−1)/Ph Fold:

Where Ph is the percentage of healthy (cleaved PARP negative) cells

Training and Test Set Subdivision

The data set for the 60 donors was split into both training and test sets. Thirty donors each were randomly assigned to the test and training set. Manual inspection of the data sets ensured that they were relatively balanced according to age and race.

### REFERENCES

1. Kornblau S M et al. (2010) Dynamic single-cell network profiles in acute myelogenous leukemia are associated with patient response to standard induction therapy. Clin. Cancer Res 16:3721-3733.
2. Rosen D B et al. (2010) Functional characterization of FLT3 receptor signaling deregulation in acute myeloid leukemia by single cell network profiling (SCNP). PLoS ONE 5:e13543.
3. Rosen D B et al. (2010) Distinct patterns of DNA damage response and apoptosis correlate with Jak/Stat and PI3kinase response profiles in human acute myelogenous leukemia. PLoS ONE 5:e12405.
4. Feinerman O et al. (2010) Single-cell quantification of IL-2 response by effector and regulatory T cells reveals critical plasticity in immune response. Mol. Syst. Biol 6:437.
5. Antony G K, Dudek A Z (2010) Interleukin 2 in cancer therapy. Curr. Med. Chem 17:3297-3302.
6. Pos Z et al. (2010) Genomic scale analysis of racial impact on response to IFN-alpha. Proc. Natl. Acad. Sci. U.S.A. 107:803-808.
7. Purvis N, Stelzer G (1998) Multi-platform, multi-site instrumentation and reagent standardization. Cytometry 33:156-165.
8. Shults K E et al. (2006) A standardized ZAP-70 assay—lessons learned in the trenches. Cytometry B Clin Cytom 70:276-283.
9. Wang L, Gaigalas A K, Yan M (2011) Quantitative fluorescence measurements with multicolor flow cytometry. Methods Mol. Biol 699:53-65.

### Example 9

### Single Cell Network Profiling (SCNP) Reveals Race-Associated Differences in B Cell Receptor Signaling Pathway Activation

Race-related differences have been documented in the incidence of autoimmune diseases such as systemic lupus erythematosus and multiple sclerosis, in the clinical response to immunotherapies [such as IFNα (in HCV infections) and belimumab (in systemic lupus erythematosus)] and to hematopoietic stem cell transplantation. However, the basis for such race-associated differences remains poorly understood. Single Cell Network Profiling (SCNP) can be a multiparametric flow cytometry based approach that can simultaneously measure intracellular signaling activity in multiple cell subpopulations. Previously, SCNP analysis of peripheral blood mononuclear cells (PBMCs) from 60 healthy donors identified a race-associated difference in αIgD induced levels of p-S6 and p-Akt in B cells. The present study extended this analysis to a broader range of signaling pathway components downstream of the B cell receptor (BCR) in European Americans and African Americans using a subset of donors from the previously analyzed cohort of 60 healthy donors.

Thirty five BCR signaling nodes (a node is defined as a paired modulator and intracellular readout) were measured by SCNP in PBMCs from 10 healthy donors [5 African Americans (36-51 yrs), 5 European Americans (36-56 yrs), all males]. Cryopreserved PBMCs were thawed, modulated at 37° C. in 96-well plates, fixed and permeabilized. Permeabilized cells were stained with fluorochrome-conjugated antibodies that recognize extracellular surface markers and intracellular signaling molecules. The levels of seven phospho-proteins [p-Lck (Y505), p-Syk (Y352), p-Akt (S473), p-S6 (S235/S236), p-p38 (T180/Y182), p-Erk (T202/Y204), and p-NFκB (S529)] were measured in CD20+ B cells at 0, 5, 15, 30, and 60 minutes post αIgD exposure. CD20 and IgD surface markers were used to determine the frequency of IgD+ B cells.

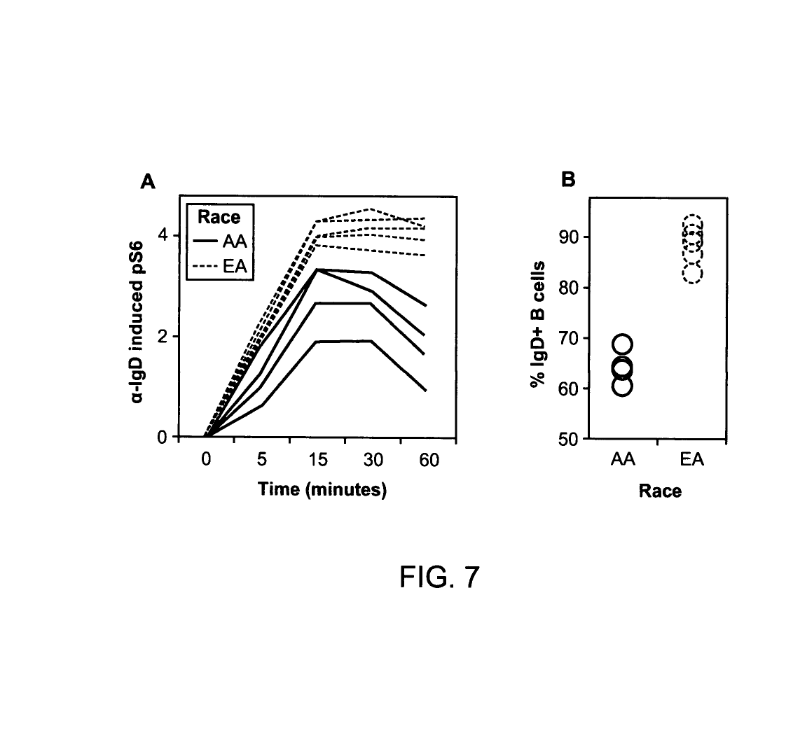

Analysis of BCR signaling activity in European American and African American PBMC samples revealed that, compared to the European American donors, B cells from African Americans had lower αIgD induced phosphorylation of multiple BCR pathway components, including the membrane proximal proteins Syk and Lck as well as proteins in the PI3K pathway (S6 and Akt), the MAPK pathways (Erk and p38), and the NFκB pathway (NFκB) (see example for αIgD induced p-S6 levels in FIG. 7A). Overall, 4 (p-Syk, p-S6, p-Akt, and p-Erk) of the 7 BCR pathway components tested (averaged over all timepoints for each donor) showed statistically significant differences in αIgD induced activation levels between racial groups (p=0.016, Wilcoxon test). Analysis of the frequency of IgD+ B cells showed that PBMCs from African Americans had a lower frequency of IgD+ B cells than PBMCs from European Americans [(p=0.016, Wilcoxon test), FIG. 7B, and that the frequency of IgD+ B cells had a strong positive correlation with BCR pathway activation (i.e. Pearson correlation coefficient r>0.6 for most BCR signaling nodes). While race-associated differences in the frequency of IgD+ B cells were detected, the levels of IgD expression (as measured by the median fluorescence intensity) in the IgD+ B cell subpopulation did not differ between the races (p=0.286). Thus, the race-related difference in BCR pathway activation is attributable, at least in part, to a race-associated difference in IgD+ B cell frequencies.

In conclusion, SCNP analysis allowed for the identification of statistically significant race-associated differences in BCR pathway activation within PBMC samples from healthy donors.

### Example 10

### Normal Non-Diseased Responses to Genotoxic Stress Using Healthy PBMC

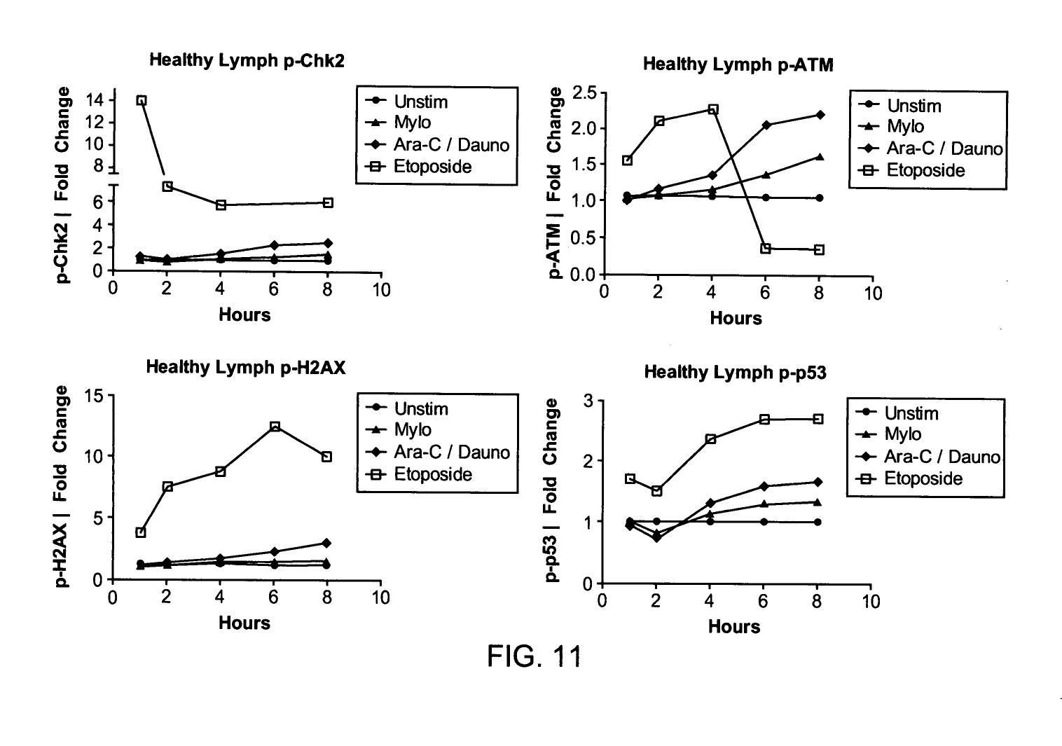

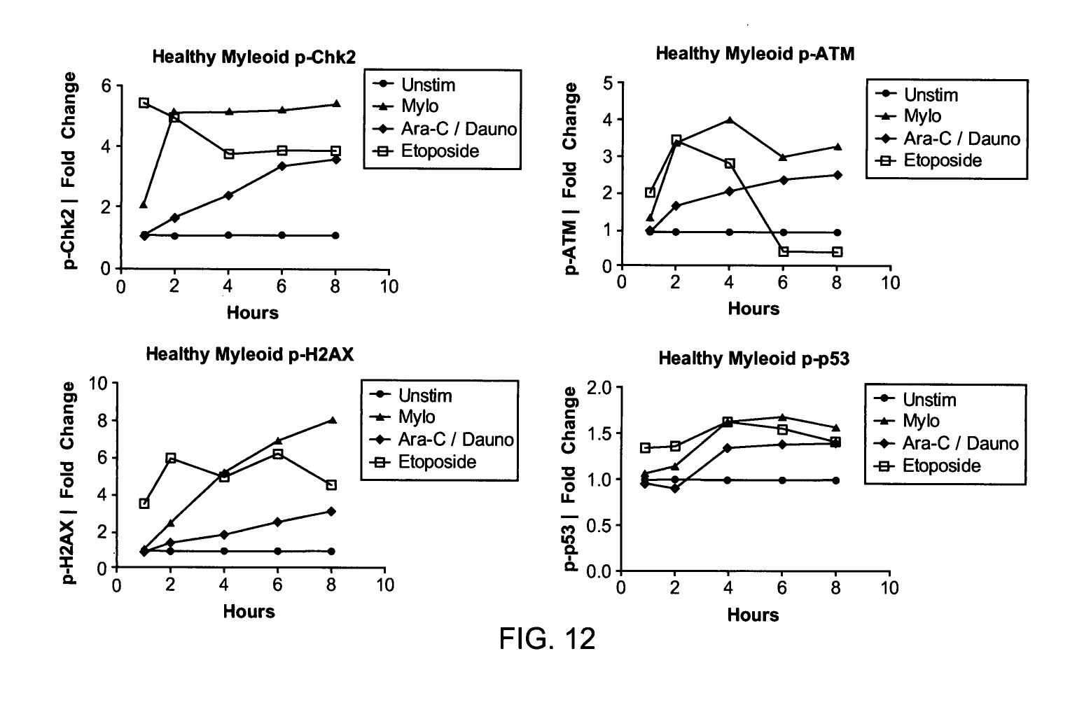

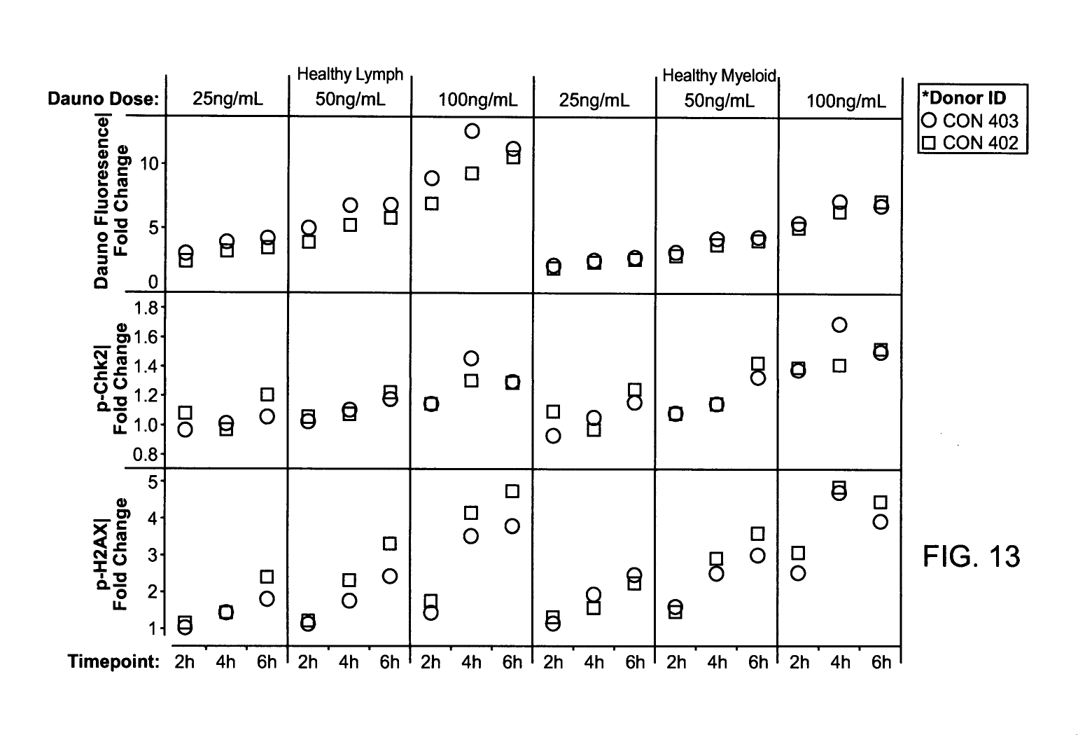

FIG. 11 shows normal PMBC DNA damage kinetics to double strand breaks induced by etoposide, Ara-C/Daunorubicin, and Mylotarg. FIG. 12 shows normal PBMC Myeloid DNA Damage Kinetics to Double Strand Breaks induced by Etoposide, Ara-C/Daunorubicin, or Mylotarg. FIG. 13 shows normal PBMC Lymph and Myeloid response to Ara-C/Daunorubicin: (kinetics and effect of Daunorubicin dose) measuring DNA Damage Response and Daunorubicin fluorescence.

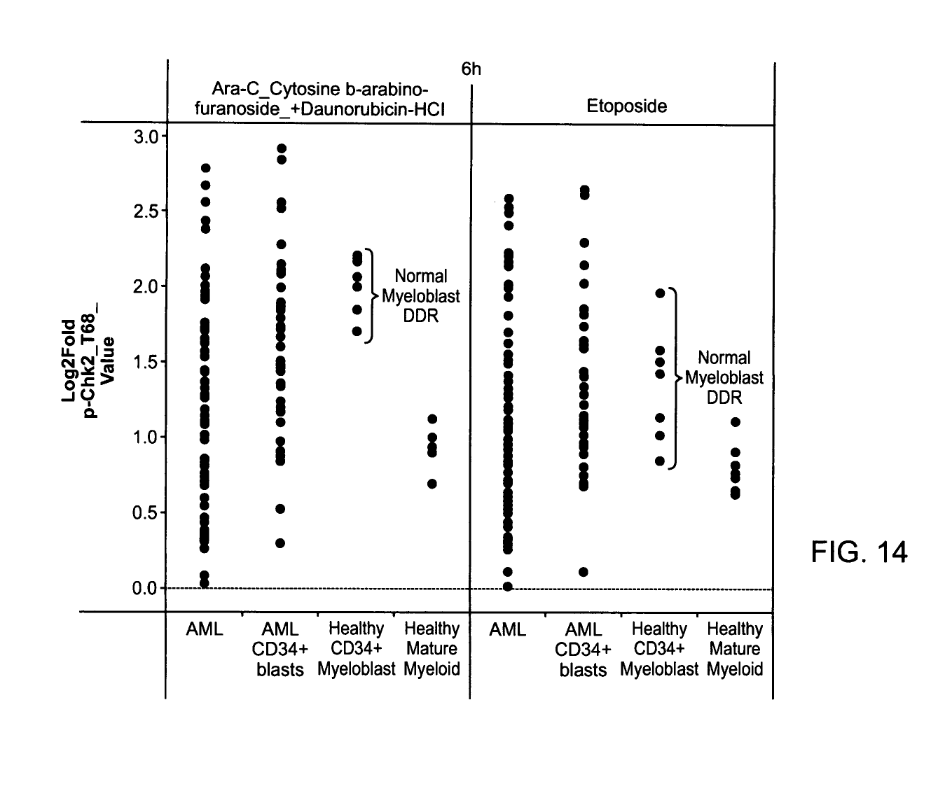

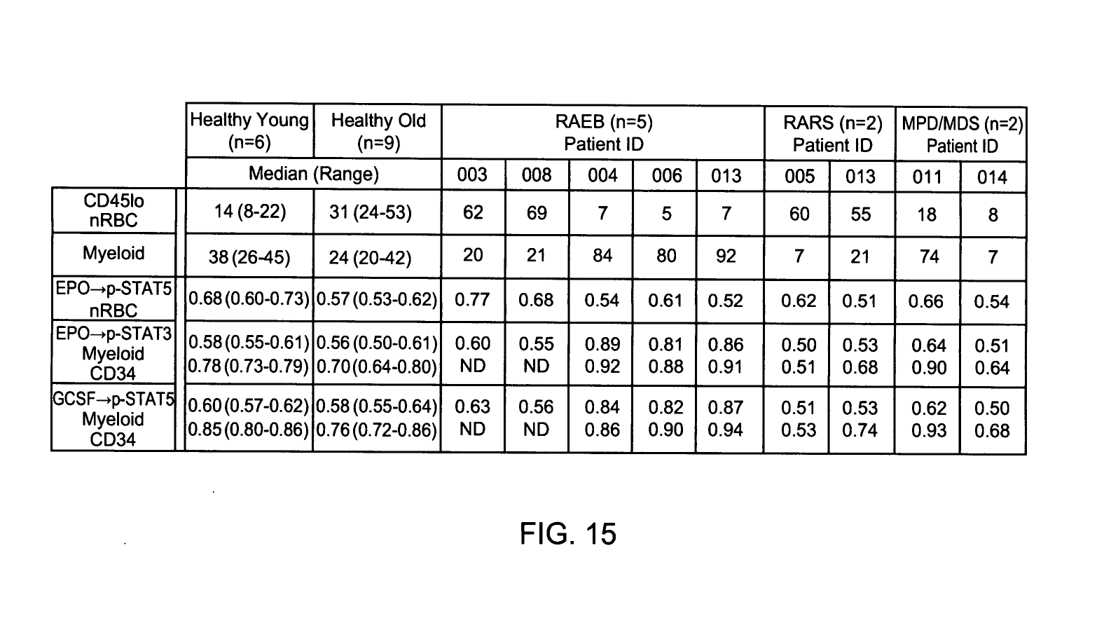

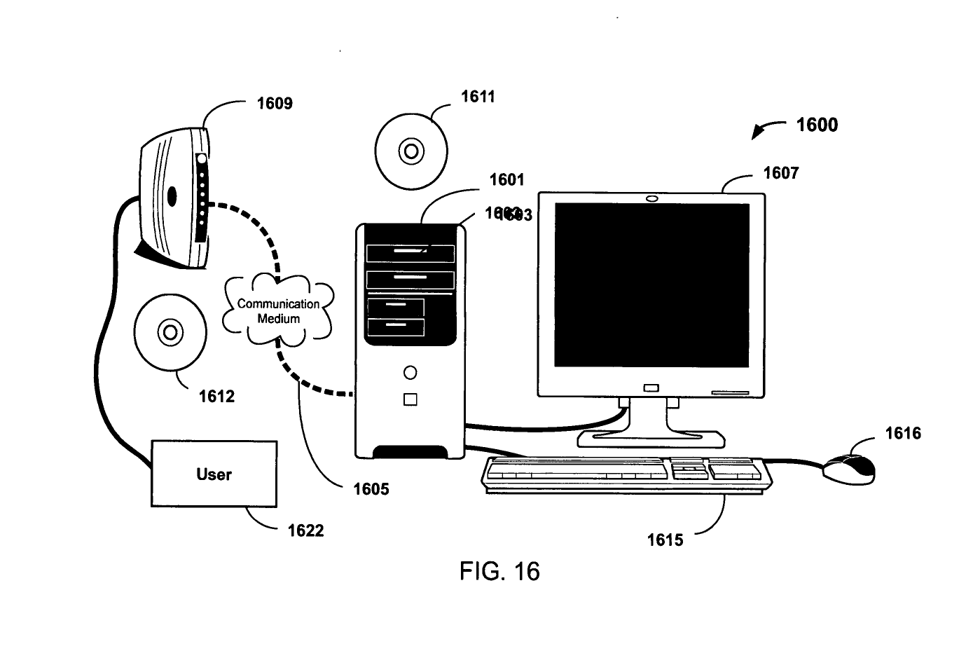

FIG. 14 shows that AML samples display a range of DDR responses compared to Normal Healthy Non-Diseased CD34+ Myeloblasts. AML DNA Damage Response (DDR) to double strand break inducing agents Ara-C/Daunorubicin or Etoposide at 6 h: AML display a range of DDR Responses; some higher than normal myeloblasts; many lower than normal myeloblasts. There is evidence of defective DDR/drug metabolism in individual patients. For example, normal Myeloblasts CD34+ display a larger induction of DDR than normal mature Myeloid cells (CD34−, D11b+). Also, CD34+ AML blasts tend to have higher DDR responses yet still display a wide range of p-Chk2 induction.

Etoposide has faster kinetics than Ara-C/Daunorubicin, Mylotarg. The peak read was around 2 hours. pATM peaks at 2 h, then diminishes significantly. pChk2 peaks at 1 h but remains detectable after 2 h. P53 and pH2AX stay at similar levels across kinetic timecourse.

After 4 h for Ara-C/Daunorubicin: a) all readouts increase with time; b) Daunorubicin Dose makes a large difference. Mylotarg (gemtuzumab ozogamicin, G0) has faster kinetics in Myeloid vs Lymphoid cells. G0 is an immunotoxin that targets CD33+ Myeloid cells. (See e.g., U.S. Patent Publication No. 20100099109). Induction of pATM, pChk2, is seen in Myeloid cells by 2 h. Some downregulation of pATM is seen at 6 and 8 h. Induction of pH2AX and p53 increase with time, and larger effects are seen after 4 h.

In summary, multiple components of DNA Damage Repair machinery can be quantified across time in normal healthy cell populations.

### Example 11

### Single Cell Network Profiling (SCNP) Reveals Age- and Disease-Based Heterogeneity in Healthy Individuals and in Patients with Low Risk (LR) Myelodysplastic Syndrome (MDS)

Background:

Normal hematopoiesis changes with age through unknown mechanisms. Low risk myelodysplasia is characterized by cytopenias arising through inefficient hematopoiesis. It was hypothesized that both of these differences might result from changes in responsiveness to external signaling. To test this, SCNP was used, a multiparametric flow cytometry-based assay that can simultaneously measure both extracellular surface marker levels and changes in intracellular signaling proteins in response to extracellular modulators, quantitatively at the single cell level (Kornblau et al. Clin Cancer Res 2010).

Methods/Objective:

SCNP was applied to examine baseline and intracellular signaling responses induced by the extracellular modulators EPO and GCSF in bone marrow (BM) mononuclear cells (BMMC) derived from healthy donors (n=15) and MDS (n=9) patients. The effects of donor age on signaling profiles in healthy BMMC was compared between samples collected by BM aspirate from 6 subjects aged 23-43 years (“younger”) and from the BM present in hip replacement samples from 9 subjects aged 54-82 years (“older”). Signaling profiles were also determined for 9 LR MDS patients aged 53-83 years and compared to the age-matched healthy “older” control. Metrics used for analysis included fold change, total phosphorylation levels, and the Mann-Whitney U statistic model.

Results:

There were no differences in the frequency of CD34+ cells (R²=0.006, p=0.78) between “younger” and “older” healthy donor samples. Likewise, there was no age-related difference in functional signaling ability in response to GCSF-induced p-STAT1, p-STAT3, & p-STAT5 levels. However, early erythroblasts and normoblasts from older healthy donors were significantly less responsive to EPO, as measured by induced phospho (p)-STAT5 levels than those derived from younger healthy donors (e.g. R²=0.654 p=0.008 for erythroblasts and R²=0.628 p=0.0004 for normoblasts). This suggests that the differences observed in EPO response were likely due to donor age rather than sample source. Signaling profiles classified RAEB patients into 2 categories based on differences in EPO- and GCSF-induced signaling (FIG. 15). Compared to healthy age-matched healthy controls, one subset was characterized by a high % of RBC precursors (CD45lo nRBC) and increased p-STAT5 levels in response to EPO and the other subset by a high % of myeloid cells with robust GCSF-induced p-STAT3 & p-STAT5 responses in both total myeloid and CD34+ cells. By contrast, patient samples with RARS had a high % of CD45lo nRBC but lacked robust p-STAT5-induced signaling after modulation with EPO.

Conclusions:

Overall, these data show the feasibility of using the SCNP assay in BM samples to functionally characterize signaling pathways simultaneously in different cell subsets of healthy donors and patients with MDS. In healthy individuals, age-related differences in EPO signaling were discovered. In LR MDS, differences in signaling were observed between cases and in comparison to the data from healthy controls. Deciphering signaling profiles in healthy donor versus MDS patient samples may result in improved, biologically-based disease classification that informs more effective patient management.

The results of this study and the approach used here have several applications. For example, by establishing a normal signaling landscape, some of the functional changes that may occur with age have been identified. This normal data set can also be used as a reference for identifying abnormal responses in diseases such as autoimmune diseases. This approach can be used to monitor changes in the immune system that occur after vaccination or with immunotherapy. Finally, this approach can be used to identify potential therapeutic targets that may allow for modulation of immune responses.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method comprising:
(a) identifying an activation level of one or more activatable elements in a first cell-type from a test sample;
(b) identifying an activation level of the one or more activatable elements in a second cell-type from a test sample;
(c) determining a similarity value based on steps a) and step b) and a statistical model, wherein the statistical model specifies a range of activation levels of one or more activatable elements in the first cell-type and the second cell-type in a plurality of normal samples, wherein the statistical model further specifies the variance of the activation levels of the one or more activatable elements associated with cells in the plurality of normal samples.

2. The method of claim 1, wherein identifying the activation level of the one or more activatable comprises:
(a) identifying the activation level of the one or more activatable elements in single cells derived from the test sample;
(b) identifying one or more cell-type markers in single cells derived from the test sample; and
(c) gating discrete populations of single cells based on the one or more cell-type markers associated with the single cells.

3. The method of claim 1, further comprising generating the statistical model, wherein generating the statistical model comprises:
(a) identifying the activation level of the one or more activatable elements in single cells derived from the plurality of normal samples;
(b) identifying one or more cell-type markers in single cells derived from the plurality of normal samples;
(c) gating cells in the plurality of normal samples based on the one or more cell-type markers associated with the single cells; and
(d) generating the statistical model that specifies the range of activation levels associated with cells in the normal samples.

4.-5. (canceled)

6. The method of claim 1, further comprising contacting the test sample and the plurality of normal samples with one or more modulators.

7.-21. (canceled)

22. The method of claim 1, further comprising administering a therapeutic agent to a subject from whom the test sample is derived based on the similarity value.

23.-44. (canceled)

45. A method of generating a normal cell profile comprising: obtaining a plurality of samples of cells from normal individuals, contacting the plurality of samples of cells from the normal individuals with one or more modulators, measuring an activation level of one or more activatable elements in the plurality of samples from the normal individuals, and generating a profile, wherein the profile comprises one or more ranges of the activation level of the one or more activatable elements from the plurality of samples of cells from the normal individuals.

46. The method of claim 45, wherein the profile comprises one or more ranges of activation levels of the one or more activatable elements that exhibit variance of less than 50% among normal samples.

47.-53. (canceled)

54. The method of claim 45, further comprising displaying the activation level of the one or more activatable elements from the plurality of samples of cells from normal individuals in a report.

55.-60. (canceled)

61. A method comprising:
(a) measuring an activation level of one or more activatable elements from cells from a test sample from a subject;
(b) comparing the activation level of the one or more activatable elements from cells from the test sample to a model, wherein the model is derived from determining a range of activation levels of one or more activatable elements from samples of cells from a plurality of normal individuals;
and
(c) preparing a report displaying the activation level of the one or activatable elements from the samples of cells from the plurality of normal individuals to the activation level of the one or more activatable elements from cells from the test sample from the subject.

62.-64. (canceled)

65. The method of claim 61, wherein the samples of cells from a plurality of normal individuals were contacted with one or more modulators

66. The method of claim 65 further comprising contacting the plurality of samples of cells from the test sample from the subject with the one or more modulators.

67.-77. (canceled)

78. The method of claim 61, further comprising making a clinical decision based on said comparing.

79. The method of claim 78, wherein the clinical decision comprises a diagnosis, prognosis, or monitoring the subject.

80. The method of claim 61, further comprising providing the report to a healthcare provider.

81. The method of claim 61, further comprising providing the report to the subject.

82. (canceled)

83. A report comprising a visual representation of multiparametric results of a test sample, the visual representation comprising a comparison between an activation level of two or more activatable elements in single cells from a test sample and a range of activation levels of the two or more activatable elements in single cells in a plurality of samples used as a standard.

84. The report of claim 83, further comprising a statistical model, wherein the statistical model specifies the range of activation levels of the two or more activatable elements in single cells in a plurality of samples used as a standard.

85. The report of claim 84, further comprising a similarity value between the activation level of the two or more activatable elements in single cells from a test sample and the statistical model.

86. The report of claim 83, further comprising a scatterplot, a line graph with error bars, a histogram, a bar and whisker plot, a circle plot, a radar plot, a heat map, and/or a bar graph.

87. The report of claim 83, wherein a computer server generates the report.

88. The report of claim 83, wherein the report comprises information on cell growth, cell survival and/or cytostasis.

89. The report of claim 83, wherein the two or more activatable elements comprise two or more proteins.

90.-94. (canceled)
